(19) United States
(12) Patent Application Publication
(10) Pub. No.: US 2006/0282916 A1
(43) Pub. Date: Dec. 14, 2006

(54) Genes controlling plant cell wall formation

(76) Inventor: Takashi Hibino, Kameyama-shi, Mie (JP)

Correspondence Address: STERNE, KESSLER, GOLDSTEIN & FOX PLLC, 1100 NEW YORK AVENUE, N.W., WASHINGTON, DC, 20005, US

(21) Appl. No.: 10/546,725

(22) PCT Filed: Feb. 24, 2004
(86) PCT No.: PCT/JP04/02151

§ 371 Date: Nov. 17, 2005

(30) Foreign Application Priority Data
Feb. 24, 2003 (JP) 200346427

(51) Int. Cl. A01H 1/00; C07H 21/04; C12N 5/04; C12N 15/82; C12N 1/21

(52) U.S. Cl. 800/284; 435/468; 435/419; 536/23.2; 435/252.2

(57) ABSTRACT

A Eucalyptus EST database was prepared and used for extracting genes specifically expressed in Eucalyptus reaction wood tissues using microarray analysis. As a result, genes were broadly classified into a gene cluster with a predominantly high expression, a gene cluster with a lower expression, and a gene cluster that is virtually unchanged, in Eucalyptus reaction wood as compared to ordinary trunks. It is thought that the gene cluster with predominantly high expression and the gene cluster with low expression can be used to control cell wall biosynthesis and wood fiber cell morphogenesis

### TECHNICAL FIELD

The present invention relates to genes that control plant cell wall biosynthesis and wood fiber cell morphogenesis, and their use.

### BACKGROUND ART

The amount of wood consumed throughout the world continues to increase each year. In terms of use, consumption of wood for fuel accounts for more than half, and this amount is on the rise in developing regions even at present. Although the amount of industrial materials, such as wood chips for lumber and paper production, consumed in developed regions has started to decrease slightly, the amounts consumed in developing countries and regions are increasing. Even though forests in developed regions have been considerably depleted due to conversion into agricultural land and use as building materials, they have recently started to increase slightly due to tree planting activities. In developing regions, however, due to commercial logging by developed countries in the past, and the increasing demand for domestic fuel and agricultural land in recent years accompanying population growth, a rapid decrease in forestland area is continuing on a global scale. As a result, problems such as global warming are occurring due to decreased ability to fix carbon dioxide. In recent years, afforestation projects are being actively conducted throughout the world in an attempt to provide a stable wood chip supply for the lumber and paper industries, while resolving these issues.

Human beings have used forest resources (wood biomass) in a diverse range of industrial fields such as papermaking, construction, animal feed, and fuel for many years. Industries that use wood biomass are being recognized anew from the viewpoint of improving global environmental issues, as resources that can be sustainably used in the future as well. Much hope is placed on these as circulatory-type industries based on the use of carbon sources as an alternative to current fossil resources. As a specific example, Japanese paper manufacturers are actively promoting afforestation projects focusing on rapidly-growing tropical trees such as Eucalyptus and acacia, in order to achieve a stable and continuous supply of wood biomass, the raw material. As an example of the scale of these afforestation projects, Oji Paper Co., Ltd. is conducting afforestation over a wide area, focusing on the Pan-Pacific region such as Oceania and SoutheastAsia, aiming at 200,000 hectares of afforested land by 2010. This clearly demonstrates that through large-scale afforestation at the commercial level, the paper industry is taking the initiative ahead of other industrial fields in recycled material production (biomass recycling) through industrial utilization and regeneration of biomass.

Along with the progress of these afforestation projects, if the amount and quality of woody cell wall components (cellulose, hemicellulose, and lignin) as well as fiber morphology (elongation of wood fiber cells) could be freely altered by artificially controlling the production of wood biomass in trees, both quantitative increases and qualitative improvements in wood biomass can be expected. This in turn is hoped to expand applications in energy usage and utilization as industrial raw materials in the future, thereby leading to replacement of current fossil materials in various fields.

Cell walls and cellulose, the main component of cell walls, play an important role in maintaining plant morphology. However, despite a considerable amount of time spent throughout the world on research to elucidate the mechanism of cellulose biosynthesis, as well genomic level analytical research on trees (Non-Patent Document 1), the details of this mechanism remain unclear. Recently, a glycosyl transferase gene, which is thought to catalyze the bonding of β-1,4 glucans serving as the basic backbone of cellulose, was reported in cotton and Arabidopsis thaliana (see, for example, Non-Patent Document 2). In addition, as a result of analyzing genes characteristic of wood formation, particularly the secondary wall synthesis of the cell wall, using microarrays for poplar vascular bundle tissues in different development stages, a known cellulose synthetase was found (Non-Patent Document 3). However, significant progress is yet to be seen in research relating to regulation of the entire cellulose biosynthesis mechanism, and the like (Patent Document 1).

[Patent Document] Japanese Patent Kohyo Publication No. (JP-A) 2002-510961 (unexamined Japanese national phase publication corresponding to a non-Japanese international publication)

[Non-patent Document 1] Genome Biol. 2002; 3(12):REVIEWS 1033.

[Non-patent Document 2] Burn et al. Plant Physiol. 129, 797-807, 2002.

[Non-patent Document 3] Hertzberg M, Aspeborg H, Schrader J, Andersson A, Erlandsson R, Blomqvist K, Bhalerao R, Uhlen M, Teeri T T, Lundeberg J, Sundberg B, Nilsson P, Sandberg G. A transcriptional roadmap to wood formation. Proc Natl Acad Sci USA. 2001 Dec. 4; 98(25):14732-7. Epub 2001 Nov. 27.

[Non-patent Document 4] Szyjanowicz P M, McKinnon I, Taylor N C, Gardiner J, Jarvis M C, Turner S R. The irregular xylem 2 mutant is an allele of korrigan that affects the secondary cell wall of Arabidopsis thaliana. Plant J. 2004 March; 37(5):730-40.

[Non-patent Document 5] Nakazono M, Qiu F; Borsuk L A, Schnable P S. Laser-capture microdissection, a tool for the global analysis of gene expression in specific plant cell types: identification of genes expressed differentially in epidermal cells or vascular tissues of maize. Plant Cell. 2003 March; 15(3):583-96. Erratum in: Plant Cell. 2003 April; 15(4):1049.

[Non-patent Document 6] Israelsson M, Eriksson M E, Hertzberg M, Aspeborg H, Nilsson P, Moritz T. Changes in gene expression in the wood-forming tissue of transgenic hybrid aspen with increased secondary growth. Plant Mol. Biol. 2003 July; 52(4):893-903.

[Non-patent Document 7] Gardiner J C, Taylor N G, Turner S R. Control of cellulose synthase complex localization in developing xylem. Plant Cell. 2003 August; 15(8):1740-8.

[Non-patent Document 8] Moller R, McDonald A G, Walter C, Harris P J. Cell differentiation, secondary cell-wall formation and transformation of callus tissue of Pinus radiata D. Don. Planta. 2003 September; 217(5):736-47. Epub 2003 Jun. 13.

[Non-patent Document 9] Joshi C P. Xylem-specific and tension stress-responsive expression of cellulose synthase genes from aspen trees. Appl Biochem Biotechnol. 2003 Spring; 105-108:17-25.

[Non-patent Document 10] Li L, Zhou Y, Cheng X, Sun J, Marita J M, Ralph J, Chiang V L. Combinatorial modification of multiple lignin traits in trees through multigene cotransformation. Proc Natl Acad Sci USA. 2003 Apr. 15; 100(8):4939-44. Epub 2003 Mar. 31.

[Non-patent Document 11] Lorenz W W, Dean J F. SAGE profiling and demonstration of differential gene expression along the axial developmental gradient of lignifying xylem in loblolly pine (Pinus taeda). Tree Physiol. 2002 April; 22(5):301-10.

[Non-patent Document 12] Demura T, Tashiro G, Horiguchi G, Kishimoto N, Kubo M, Matsuoka N, Minami A, Nagata-Hiwatashi M, Nakamura K, Okamura Y, Sassa N, Suzuki S, Yazaki J, Kikuchi S, Fukuda H. Visualization by comprehensive microarray analysis of gene expression programs during transdifferentiation of mesophyll cells into xylem cells. Proc Natl Acad Sci USA. 2002 Nov. 26; 99(24):15794-9. Epub 2002 Nov. 18.

[Non-patent Document 13] Aharoni A, Keizer L C, Van Den Broeck H C, Blanco-Portales R, Munoz-Blanco J, Bois G; Smit P, De Vos R C, O'Connell A P. Novel insight into vascular, stress, and auxin-dependent and-independent gene expression programs in strawberry, a non-climacteric fruit. Plant Physiol. 2002 July; 129(3):1019-31.

### DISCLOSURE OF THE INVENTION

Japan lacks natural resources and is dependent on fossil resources such as petroleum and natural gas even now. In order to change these circumstances using new technology, the recycling of trees (wood biomass) is currently considered to be instrumental. Although foreign countries differed in their approach towards forestry in the past, the establishment of technologies relating to the effective use of wood biomass was placed as an important topic at the beginning of the current century. In fact, several countries have begun research on target wood species (pine trees of needle-leaved trees and poplar of broad-leaved trees in the U.S., spruce and poplar in Canada, and poplar in Scandinavia) using genomic analyses as national projects. It is well known that the U.S. and Europe are currently ahead of research on basic technologies related to gene recombination of important crop varieties involved in food production. Learning from this, there is an extremely high need to identify genes that control plant cell wall component biosynthesis and wood fiber cell morphogenesis, so that Japan can become the technological powerhouse it was before, or to at least keep up with foreign countries, and be involved in the production of recycled material through the utilization of wood biomass on a global scale.

Considering the aforementioned circumstances, an objective of the present invention is to provide genes that control plant cell wall component biosynthesis and wood fiber cell morphogenesis, plasmids comprising these genes, and plant cells, microorganisms, or plants transformed by the plasmids.

As a result of extensive research to achieve the aforementioned objective, the present inventor concluded that the acquisition of a gene cluster that controls wood biomass formation and comprehensive analyses relating to its expression and function should be carried out based on a genomic approach. Namely, it was concluded that it is desirable to use a method of systematically acquiring and analyzing a target gene cluster at a time when formation of a specific tissue (particularly the cell wall) is active and cellulose is specifically biosynthesized. Moreover, it was concluded that, in order to provide a plant with characteristics useful for human use by artificially controlling the expression of an altered gene using genetic engineering technology, it is necessary to identify a gene cluster specific to various tissues that selectively express the novel characteristics in suitable plant tissues.

In the implementation of this research, resources for analyses were prepared using Eucalyptus. More specifically, various gene libraries and an EST database were prepared for each of trunk, leaf, and root tissues. Gene libraries and mutants involved in cell wall biosynthesis were already present for Arabidopsis thaliana, a plant that is widely used as a plant model throughout the world. These causative genes have already been analyzed.

The present inventor extracted genes specifically expressed in Eucalyptus reaction wood forming tissue by microarray analysis, using the aforementioned Eucalyptus EST database. As a result, the genes were broadly classified into a gene cluster demonstrating predominantly high expression, a gene cluster demonstrating low expression, and a gene cluster which demonstrated virtually no changes in expression, in Eucalyptus reaction wood as compared with ordinary trunk wood. The gene cluster that demonstrated predominantly high expression and the gene cluster that demonstrated low expression may be used to control cell wall component biosynthesis and wood fiber cell morphgenesis. In particular, the gene cluster that demonstrated predominantly high expression, may be involved in cell wall component biosynthesis and wood fiber cell morphogenesis, and may be used to promote cell wall component biosynthesis and wood fiber cell morphogenesis.

One of the outcomes of the gene clusters that control cell wall component biosynthesis and wood fiber cell morphogenesis obtained by the present invention, as well as techniques for their overall control, would be various quantitative and qualitative changes (such as high cellulose content, low lignin content, thick or thin cell walls, and long or short fiber lengths) in the characteristics of novel transgenic Eucalyptus varieties obtained by using these gene clusters.

Namely, the present invention relates to genes that control plant cell wall biosynthesis and wood fiber cell motphogenesis, and provides the following [1] to [14].

[1] A DNA whose expression increases during plant cell wall biosynthesis and wood fiber cell morphogenesis, wherein the DNA is described in (a) or (b) below:

(a) a DNA that hybridizes under stringent conditions with a DNA comprising a nucleotide sequence described in any one of SEQ ID NOs: 1 to 862; or,
(b) a DNA encoding a protein having 50% or more homology with a protein comprising an amino acid sequence encoded by the DNA of (a).
   [2] The DNA of [1], wherein expression increases in plant reaction wood forming tissue.
   [3] A DNA whose expression decreases during plant cell wall biosynthesis and wood fiber cell morphogenesis, wherein the DNA is described in (a) or (b) below:
(a) a DNA that hybridizes under stringent conditions with a DNA comprising a nucleotide sequence described in any one of SEQ ID NOs: 863 to 1731; or,
(b) a DNA encoding a protein having 50% or more homology with a protein comprising an amino acid sequence encoded by the DNA of (a).
   [4] The DNA of [3], wherein expression decreases in plant reaction wood forming tissue.
   [5] The DNA of any one of [1] to [4], wherein the plant is Eucalyptus.
   [6] A DNA encoding a protein comprising an amino acid sequence in which one or more nucleotides are substituted, deleted, added and/or inserted in an amino acid sequence encoded by the DNA of any one of [1]to [5].
   [7] A promoter DNA of the DNA of any one of [1] to [5].
   [8] A DNA described in any one of (a) to (e) below:
(a) a DNA encoding an antisense RNA complementary to a transcription product of the DNA of any one of [1] to [5];
(b) a DNA encoding an RNA having ribozyme activity that specifically cleaves a transcription product of the DNA of any one of [1] to [5];
(c) a DNA encoding an RNA that suppresses expression of the DNA of any one of [1] to [5] by RNAi effects;
(d) a DNA encoding an RNA that suppresses expression of the DNA of any one of [1] to [5] by co-suppression effects; and,
(e) a DNA encoding a protein having a dominant negative trait against a transcription product of the DNA of any one of claims [1] to [5].
   [9] A recombinant vector comprising the DNA of any one of [1] to [6] or [8].
   [10] A microorganism retaining a plasmid comprising the promoter DNA of [7] or the vector of [9].
   [11] A transgenic plant cell introduced with the vector of [9].
   [12] A transgenic plant that is re-differentiated from the transgenic plant cell of [11].
   [13] A transgenic plant that is a progeny or a clone of the transgenic plant of [12].
   [14] A breeding material of the transgenic plant of [12] or [13].

The present inventor discovered DNA with varied expression in Eucalyptus reaction wood forming tissue. Cell wall component biosynthesis and wood fiber cell morphogenesis are known to take place in plant reaction wood forming tissue (although general descriptions on reaction wood can always be found in technical literature relating to wood, a paper by Baba, et al. (Mokuzai Gakkaishi 42, 795-798, 1996) describes detailed data on the chemical and structural properties of reaction wood in Eucalyptus). Based on the aforementioned findings, the present invention provides DNA whose expression varies during plant cell wall component biosynthesis and wood fiber cell morphogenesis.

A DNA whose expression varies during plant cell wall component biosynthesis and wood fiber cell morphogenesis of the present invention may be used to control plant cell wall component biosynthesis and wood fiber cell morphogenesis. In addition, a DNA whose expression increases during plant cell wall component biosynthesis and wood fiber cell morphogenesis is particularly thought to be involved in plant cell wall component biosynthesis and wood fiber cell morphogenesis, and therefore may be used to promote cell wall component biosynthesis and wood fiber cell morphogenesis. On the other hand, a DNA whose expression decreases during plant cell wall component biosynthesis and wood fiber cell morphogenesis may be involved in the control of tissue-specific or time-specific expression, by basically suppressing genes involved in cell wall component biosynthesis and wood fiber cell morphogenesis through some sort of a mechanism. Thus, these DNAs may be used to enhance cell wall component biosynthesis and wood fiber cell morphogenesis by artificially prompting their decrease.

Controlling cell wall component biosynthesis and wood fiber cell morphogenesis in plants has various important significances in industrial and agricultural fields. For example, alteration of plant cell wall components is significant in terms of economical efficiency and profitability, by enhancing the supply of high-quality fiber raw materials such as pulp as a result of increasing cellulose and hemicellulose contents, and by improving the digestion and absorption efficiencies of useful agricultural crops and feed products. In addition, changing the structure of polysaccharides, which is a cell wall component, may lead to the production of raw material plants having new industrial values. Moreover, alteration of cell morphology is significant in terms of, for example, improving the fiber characteristics of fiber raw materials such as pulp.

In addition, a DNA of the present invention whose expression varies during plant cell wall component biosynthesis and wood fiber cell morphogenesis can also be used as a specific marker for identifying cells and tissues in which cell wall component biosynthesis and wood fiber cell morphogenesis are taking place.

There are no particular limitations on the plants from which the DNA of the present invention is derived from. Examples include useful agricultural crops such as grains, vegetables, and fruits (including feed crops), fiber raw material plants such as pulp, and plants valued for their aesthetic beauty such as foliage plants. There are no particular limitations on such plants, and examples include Eucalyptus, pine, acacia, poplar, cedar, cypress, bamboo, yew, rice, corn, wheat, barley, rye, potato, tobacco, sugar beet, sugar cane, rapeseed, soybean, sunflower, cotton, orange, grape, peach, pear, apple, tomato, Chinese cabbage, cabbage, radish, carrot, squash, cucumber, melon, parsley, orchid, chrysanthemum, lily, and saffron.

An example of a DNA of the present invention is a DNA that hybridizes under stringent conditions with a DNA comprising a nucleotide sequence described in any one of SEQ ID NOs: 1 to 1731. Among these, a DNA that hybridizes under stringent conditions with a DNA comprising a nucleotide sequence described in any one of SEQ ID NOs: 1 to 862 is a DNA whose expression increases during plant cell wall component biosynthesis and wood fiber cell morphogenesis. In addition, a DNA that hybridizes under stringent conditions with a DNA comprising a nucleotide sequence described in any one of SEQ ID NOs: 863 to 1731 is a DNA whose expression decreases during plant cell wall component biosynthesis and wood fiber cell morphogenesis.

Stringent hybridization conditions comprise allowing to stand overnight at 60° C. in 0.1×SSC solution, or conditions yielding stringencies similar to these. Under these conditions, a DNA that hybridizes with a DNA comprising a nucleotide sequence described in any one of SEQ ID NOs: 1 to 1731 can be isolated.

More specifically, a continuous proximal sequence can be easily acquired by extracting a DNA from a plant, constructing a gene library, and screening under similar conditions, or by carrying out the TAIL-PCR method established by Ryu, et al. on the extracted DNA using an arbitrary sequence of about 20 mer from a 60 mer sequence (nucleotide sequence described in any one of SEQ ID NOs: 1 to 1731) for the primer.

In addition, the present invention provides a DNA that encodes a protein having 50% or more homology with a protein comprising an amino acid sequence encoded by a DNA that hybridizes under stringent conditions with a DNA comprising a nucleotide sequence described in any one of SEQ ID NOs: 1 to 1731. Such DNA can be isolated by methods commonly known to persons skilled in the art. Examples include, methods that use hybridization technology (Southern, E M., J Mol Biol, 1975, 98, 503) or polymerase chain reaction (PCR) technology (Saiki, R K et al., Science, 1985, 230, 1350., Saiki, RK. et al., Science, 1988, 239, 487). Namely, isolation of a DNA having high homology with a DNA that hybridizes under stringent conditions with a DNA comprising a nucleotide sequence described in any one of SEQ ID NOs: 1 to 1731 from a plant by using a DNA or a portion thereof that hybridizes under stringent conditions with a DNA comprising a nucleotide sequence described in any one of SEQ ID NOs: 1 to 1731 as a probe, or by using an oligonucleotide that specifically hybridizes with a DNA that hybridizes under stringent conditions with a DNA comprising a nucleotide sequence described in any one of SEQ ID NOs: 1 to 1731 as a primer, are tasks that can be routinely carried out by one skilled in the art.

Hybridization reactions to isolate such DNAs are preferably conducted under stringent conditions. Stringent hybridization conditions of the present invention include conditions such as 6 M urea, 0.4% SDS, and 0.5×SSC, and those conditions yielding similar stringencies to these. DNAs with higher homology are expected to be isolated when hybridization is performed under more stringent conditions, for example, 6 M urea, 0.4% SDS, and 0.1×SSC. DNAs thus isolated are thought to have high homology, at an amino acid level, with amino acid sequences encoded by DNAs that hybridize under stringent conditions to DNAs comprising any one of the nucleotide sequences described in SEQ ID NOs: 1 to 1731. Herein, high homology means an identity over the entire amino acid sequence of at least 50% or above, more preferably 70% or above, even more preferably 80% or above, still more preferably 90% or above, even still more preferably 95% or above, and most preferably 98% or above. Such DNAs comprise degenerative variants of the DNAs that hybridize under stringent conditions with the DNAs comprising any one of the nucleotide sequences described in SEQ ID NOs: 1 to 1731.

The degree of homology of one amino acid sequence or nucleotide sequence to another can be determined using the BLAST algorithm by Karlin and Altschul (Proc. Natl. Acad. Sci. USA, 1990, 87, 2264-2268., Karlin, S. & Altschul, S F., Proc. Natl. Acad. Sci. USA, 1993, 90, 5873). Programs such as BLASTN and BLASTX, developed based on the BLAST algorithm (Altschul, SF. et al., J. Mol. Biol., 1990, 215, 403.), are also used. To analyze a nucleotide sequence according to BLASTN, parameters are set, for example, at score=100 and word length=12. On the other hand, parameters used for the analysis of amino acid sequences by BLASTX are, for example, score=50 and word length=3. The default parameters for each program are used when using the BLAST and Gapped BLAST programs. Specific techniques of such analyses are known in the art (see http://www.ncbi.nlm.nih.gov.)

In addition, the DNA of the present invention comprises a DNA that encodes a protein comprising an amino acid sequence encoded by a DNA that hybridizes under stringent conditions with a DNA comprising a nucleotide sequence described in any one of SEQ ID NOs: 1 to 1731, or an amino acid sequence in which one or more amino acids are substituted, deleted, added and/or inserted in an amino acid sequence having 50% or more homology with a protein comprising the amino acid sequence.

An example method widely known to persons skilled in the art for preparing the aforementioned DNA is a method in which a mutation is introduced into a DNA by site-directed mutagenesis (Kramer, W. & Fritz, H J., Methods Enzymol, 1987, 154, 350).

Modification of amino acids in proteins is usually in the range of not more than 50 in the whole number of amino acids, preferably not more than 30, more preferably not more than 10, and even more preferably, not more than 3 amino acids. Amino acid modifications may be performed, for example, in the case of mutations and substitutions, using a “Transformer Site-directed Mutagenesis Kit” or “ExSite PCR-Based Site-directed Mutagenesis Kit” (Clontech), and, in the case of deletions, using a “Quantum leap Nested Deletion Kit” (Clontech) and the like.

A nucleotide sequence may be mutated without causing mutations in the amino acids within a protein (degenerative mutation). The present invention also comprise such degenerative mutant DNAs.

There is no particular limitation on the type of DNAs of this invention as long as they are capable of encoding the proteins of this invention, and include genomic DNA, cDNA, chemically synthesized DNA, etc. Genomic DNAs may be prepared by conducting PCR (Saiki et al., Science, 1988, 239, 487) using as a template genomic DNA prepared according to a method described in literature (Rogers and Bendich, Plant Mol. Biol., 1985, 5, 69) and primers prepared based on a nucleotide sequence of a DNA of this invention (e.g. a nucleotide sequence set forth in any one of SEQ ID NOs: 1 to 1731). Furthermore, cDNA may be prepared according to the standard method (Maniatis et al., “Molecular Cloning”, Cold Spring Harbor Laboratory Press), by preparing mRNA from plants, performing reverse transcription, and conducting PCR using primers similar to those described above. Genomic DNA and cDNA may also be prepared by constructing a genomic DNA library or a cDNA library according to the standard method, and screening this library using a probe, for example, one synthesized based on the a nucleotide sequence of a DNA of the present invention (e.g. the sequence set forth in any one of SEQ ID NOs: 1 to 1731). The DNA thus obtained may be easily sequenced using, for example, the “Sequencer Model 373” (ABI).

In addition, the present invention provides a promoter DNA of a DNA of the present invention. Such promoter DNA include a promoter DNA adjoining a gene that is specifically expressed by a plant (particularly a tree) obtained according to the present invention, during cell wall formation and/or specifically expressed during cellulose biosynthesis. Here, “promoter DNA” refers to a DNA comprising a specific nucleotide sequence required to start mRNA synthesis (transcription) using DNA as a template, and comprises a DNA present in nature, as well as a DNA produced by recombination or other artificial modification procedure.

A promoter of the present invention can be produced and used as described below. A DNA is extracted and purified from the tissue of a target Eucalyptus plant. Various methods can be used for the DNA preparation, including commercial kits such as the ISOPLANT Kit (Nippon Gene).

An oligonucleotide can then be produced from two arbitrary locations based on the nucleotide sequence of Eucalyptus cDNA that has already been successfully isolated by the present inventor using the resulting DNA as material. Genomic DNA corresponding to the selected Eucalyptus cDNA can then be easily produced by PCR using this oligonucleotide as primer. An upstream DNA of the gene can be isolated by PCR using an oligonucleotide primer produced based on the nucleotide sequence of the gene (Inverse-PCR and Anchor PCR/TAIL PCR (Shimamoto Ko, et al., ed., “Shinpan Shokubutsuno PCR Jikken Protocol (PCR Experimental Protocols for Plants, New edition)” (Bessatu Shokubutsu Saibou Kogaku (Plant Cell Technology Supplementary Volume), Shokubutsu Saibou Kogaku (Plant Cell Technology) Series 7), Shujunsha Co., Ltd., July 1997)), or by hybridization using a DNA sequence of the gene as probe.

A genomic DNA library can also be used for the Eucalyptus DNA. A genomic DNA library is obtained by inserting a DNA extracted from Eucalyptus into a cloning vector, such as various types of vectors derived from λDNA, cosmid vector, or TAC vector (Liu, et al. (1999), Proc. Natl. Acad. Sci. USA, Vol. 96, p. 6535), and then transforming Escherichia coli.

Hybridization techniques can be used to screen the genomic DNA library. A Eucalyptus cDNA sequence successfully isolated previously by the present inventor can be used for the probe. A clone comprising a DNA sequence homologous to the gene is isolated by screening the aforementioned DNA library using this probe. The structure of the cloned DNA is determined by producing a restriction enzyme cleavage map, determining nucleotide sequences and so forth, to specify the sequence present upstream from the gene. This upstream sequence preferably contains a TATA box sequence, and is at least several hundred bp to several kbp in size. This sequence is then cut out by a suitable restriction enzyme, and sub-cloned to other plasmid vectors and so forth as necessary.

The promoter activity of the aforementioned sequence can be analyzed as described below. For example, a vector such as pBI101 comprising a reporter gene is used, wherein the aforementioned sequence is subcloned such that it is linked upstream of the reporter gene. E. coli β-glucuronidase (GUS) is used as the reporter gene in the pBI101 vector. Gene expression can be monitored at the tissue level by using 5-bromo-4-chloro-3-β-D-glucuronic acid (X-gluc) as substrate, since a gene product of indigotin is formed as a blue precipitate as a result of substrate degradation. In addition, if 4-methyl-umbelliferyl-β-D-glucuronide (4MUG) is used for the substrate, gene expression can be quantified according to the fluorescence produced by the gene product. Furthermore, chloramphenicol acetyl transferase gene, luciferase gene, green fluoroscein protein gene and so forth can also be used for the reporter gene, in addition to the GUS gene.

A chimeric gene construct produced as described above can be introduced into, for example, a plant such as Arabidopsis thaliana mediated by an Agrobacterium, to analyze its function. When using pBI101 for the vector, a recombinant plasmid comprising the chimeric gene is introduced into, for example, Agrobacterium tumefaciens strain MP90 using electroporation, and the resulting transformant is infected into an Arabidopsis thaliana plant by, for example, the floral dip method (Shimamoto Ko et al., ed., “Model Shokubutuno Jikken Protocol (Experimental Protocols for Model Plants)” (Bessatu Shokubutu Saibou Kogaku (Plant Cell Technology Supplementary Volume), Shokubutu Saibou Kogaku (Plant Cell Technology) Series 4), Shujunsha Co., Ltd., April, 1996)). Seeds from the infected plant are seeded in medium containing agents such as kanamycin based on the vector used, to obtain a transgenic plant that has become drug-resistant as a result of gene introduction. Expression of the GUS reporter gene is then analyzed using this transgenic plant. A promoter of the present invention or an expression vector comprising the same, can be used as described below. A desired gene downstream from a promoter of the present invention (e.g. a chimeric gene linked to a gene involved in a certain type of response to osmotic pressure stress) is inserted into, for example, a pBI101 vector to construct an expression vector. This vector is then introduced into, for example, a tobacco plant mediated by Agrobacterium. The resulting transgenic plant is expected to be able to grow even under salt damage or in dry areas, as a result of gene expression in roots subjected to an environment with osmotic pressure stress, due to the action of the promoter of the present invention. In this case, unlike the 35S promoter, it is expected that other undesirable traits will not emerge, because gene expression in unwanted tissues will not occur.

Genes that can be controlled with a promoter of the present invention are not limited to the aforementioned specific gene. In addition, the function of a promoter of the present invention can be altered by coupling another expression regulating sequence to a promoter of the present invention. Examples of such expression regulating sequences include enhancer sequences, repressor sequences, and insulator sequences. A promoter of the present invention comprises several cis-element sequences that control the expression of genes involved in trunk-specificity and cell wall biosynthesis as functional characteristics. A portion of a promoter of the present invention can be inserted into and coupled with another promoter to alter the function of that promoter, with the aim of utilizing a cis-element sequence comprised in a promoter of the present invention.

In addition, the present invention provides a DNA for suppressing the expression of a DNA encoding a protein that controls plant cell wall component biosynthesis and wood fiber cell morphogenesis. Preferred embodiments of DNA for suppressing the expression of an endogenous gene can be exemplified by a DNA that encodes an antisense RNA complementary to a transcription product of a DNA of the present invention, a DNA that encodes an RNA having ribozyme activity that specifically cleaves a transcription product of a DNA of the present invention, a DNA that encodes an RNA that suppresses expression of a DNA of the present invention by RNAi effects or co-suppression effects, and a DNA that encodes a protein having dominant native trait against a transcription product of a DNA of the present invention. The aforementioned “suppressing the expression of an endogenous gene” comprise suppression of gene transcription and/or suppression of translation to a protein encoded by the gene. In addition, it also comprises not only the complete cessation of gene expression, but also a decrease in expression.

Antisense techniques are the most commonly used methods in the art to suppress the expression of a specific endogenous gene in plants. Ecker et al. were the first to demonstrate the antisense effect of an antisense RNA introduced into plant cells by electroporation (Ecker, JR. & Davis, R W., Proc. Natl. Acad. Sci. USA, 1986, 83, 5372). Thereafter, it was reported that the expression of antisense RNAs reduced target gene expression in tobacco and petunias (van der Krol A R. et al., Nature, 1988, 333, 866.). Antisense techniques have now been established as a means for suppressing target gene expression in plants.

Multiple factors act in the suppression of target gene expression by antisense nucleic acids. These include: inhibition of transcription initiation by triple strand formation; inhibition of transcription by hybrid formation at a site where the RNA polymerase has formed a local open loop structure; transcription inhibition by hybrid formation with the RNA being synthesized; inhibition of splicing by hybrid formation at an intron-exon junction; inhibition of splicing by hybrid formation at a site of spliceosome formation; inhibition of mRNA translocation from the nucleus to the cytoplasm by hybrid formation with mRNA; inhibition of splicing by hybrid formation at a capping site or poly A addition site; inhibition of translation initiation by hybrid formation at a translation initiation factor binding site; inhibition of translation by hybrid formation at a ribosome binding site near the initiation codon; inhibition of peptide chain elongation by hybrid formation in a translated region or at an mRNA polysome binding site; and inhibition of gene expression by hybrid formation at a site of interaction between nucleic acids and proteins. These antisense nucleic acids suppress target gene expression by inhibiting various processes such as transcription, splicing, or translation (Hirashima and Inoue, “Shin Seikagaku Jikken Koza (New Biochemistry Experimentation Lectures) 2, Kakusan (Nucleic Acids) IV, Idenshi No Fukusei To Hatsugen (Replication and Expression of Genes),” Nihon Seikagakukai Hen (The Japanese Biochemical Society), Tokyo Kagaku Dozin, pp. 319-347, (1993)).

The antisense sequences of the present invention can suppress target gene expression by any of the above mechanisms. In one embodiment, an antisense sequence designed to be complementary to an untranslated region near the 5′ end of the mRNA of a gene is thought to effectively inhibit translation of that gene. Sequences complementary to coding regions or to an untranslated region on the 3′ side can also be used. Thus, the antisense DNAs used in the present invention include both DNAs comprising antisense sequences against untranslated and translated regions of the gene. The antisense DNAs to be used are conjugated downstream of an appropriate promoter, and are preferably conjugated to sequences containing the transcription termination signal on the 3′ side. DNAs thus prepared can be transformed into a desired plant by known methods. The sequences of the antisense DNAs are preferably sequences complementary to an endogenous gene of the plant to be transformed, or a part thereof, but need not be perfectly complementary so long as they can effectively suppress the gene's expression. The transcribed RNAs are preferably at least 90%, and more preferably at least 95% complementary to the transcribed product of the target gene. In order to effectively suppress the expression of a target gene by means of an antisense sequence, antisense DNAs should be at least nucleotides long, more preferably at least 100 nucleotides long, and still more preferably at least 500 nucleotides long. However, the antisense DNAs to be used are generally shorter than 5 kb, and preferably shorter than 2.5 kb.

DNAs encoding ribozymes can also be used to suppress the expression of endogenous genes. A ribozyme is an RNA molecule comprising catalytic activity. There are many ribozymes comprising various activities, and among them, research focusing on ribozymes as RNA-cleaving enzymes has enabled the design of ribozymes that cleave RNAs site-specifically. While some ribozymes of the group I intron type or the M1 RNA contained in RNaseP consist of 400 nucleotides or more, others belonging to the hammerhead-type or the hairpin-type comprise an activity domain of about 40 nucleotides (Makoto Koizumi and Eiko Ohtsuka, Tanpakushitsu Kakusan Kohso (Nucleic acid, Protein, and Enzyme), 1990, 35, 2191).

The self-cleavage domain of a hammerhead-type ribozyme cleaves at the 3′ side of C15 of the G13U14C15 sequence, and formation of a nucleotide pair between U14 and A9 at the ninth position is considered to be important for this ribozyme activity. It has been shown that cleavage may also occur when the 15th nucleotide is A15 or U15 instead of C15 (Koizumi, M. et al., FEBS Lett, 1988, 228, 228.). If a ribozyme is designed to comprise a substrate-binding site complementary to the RNA sequences adjacent to the target site, one can create a restriction-enzyme-like RNA-cleaving ribozyme which recognizes the UC, UU, or UA sequence within a target RNA (Koizumi M. et al., FEBS Lett, 1988, 239, 285; Makoto Koizumi and Eiko Ohtsuka, Tanpakushitsu Kakusan Kohso (Nucleic acid, Protein, and Enzyme), 1990, 35, 2191; Koizumi M. et al., Nucleic Acids Res., 1989, 17, 7059). For example, in the coding region of DNAs that encode proteins that control plant cell wall component biosynthesis and wood fiber cell morphogenesis, there are a number of sites that can be used as targets.

Hairpin-type ribozymes are also useful in the present invention. These ribozymes can be found, for example, in the minus strand of satellite RNA in tobacco ringspot virus (Buzayan J M., Nature, 1986, 323, 349). Ribozymes that cleave RNAs target-specifically have also been shown to be produced from hairpin-type ribozymes (Kikuchi Y & Sasaki N., Nucleic Acids Res, 1991, 19, 6751; Yo Kikuchi, Kagaku To Seibutsu (Chemistry and Biology), 1992, 30, 112.).

Transcription is enabled in plant cells by fusing a ribozyme, designed to cleave a target, with a promoter such as the cauliflower mosaic virus 35S promoter, and with a transcription termination sequence. If extra sequences have been added to the 5′ end or the 3′ end of the transcribed RNA, ribozyme activity can be lost. In such cases, one can place an additional trimming ribozyme, which functions in cis, on the 5′ or the 3′ side of the ribozyme portion, in order to precisely cut the ribozyme portion from the transcribed RNA containing the ribozyme (Taira, K. et al., Protein Eng, 1990, 3, 733., Dzianott, A M. & Bujarski, J J., Proc Natl Acad Sci USA, 1989, 86, 4823., Grosshans, C A. & Cech, T R., Nucl Acids Res, 1991, 19, 3875., Taira, K. et al., Nucl Acids Res, 1991, 19, 5125.). Even greater effects can be achieved by arranging these structural units in tandem, enabling multiple sites within a target gene to be cleaved (Yuyama, N. et al., Biochem Biophys Res Commun, 1992, 186, 1271.). Thus, using these ribozymes, the transcription products of a target gene of the present invention can be specifically cleaved, thereby suppressing expression of the gene.

Endogenous gene expression can also be suppressed by RNA interference (RNAi), using double-stranded RNAs that comprise a sequence identical or similar to a target gene. RNAi refers to the phenomenon in which a double-stranded RNA comprising a sequence identical or similar to a target gene sequence is introduced into cells, thereby suppressing expression of both the exogenous gene introduced and the target endogenous gene. The details of the RNAi mechanism are unclear, but it is thought that an introduced double-stranded RNA is first degraded into small pieces, which somehow serve as a target gene indicator, resulting in degradation of the target gene. RNAi is known to be effective in plants as well (Chuang, CF. & Meyerowitz, E M., Proc Natl Acad Sci USA, 2000, 97, 4985.). For example, in order to use RNAi to suppress the expression of DNAs encoding the proteins that control plant cell wall component biosynthesis and wood fiber cell morphogenesis in plants, nucleotide sequences described in any one of SEQ ID NOs: 1 to 1731, or double-stranded RNAs comprising a sequence similar to these DNAs, can be introduced into the plants in question. Genes used for RNAi need not be completely identical to a target gene; however, they should comprise sequence identity of at least 70% or above, preferably 80% or above, more preferably 90% or above, and most preferably 95% or above. Sequence identity can be determined by an above-described method.

Suppression of endogenous gene expression can be achieved by co-suppression, through transformation with a DNA comprising a sequence identical or similar to a target gene sequence. “Co-suppression” refers to the phenomenon wherein transformation is used to introduce plants with a gene comprising a sequence identical or similar to a target endogenous gene sequence, thereby suppressing expression of both the exogenous gene introduced and the target endogenous gene. Although the details of the co-suppression mechanism are unclear, at least a part is thought to overlap with the RNAi mechanism. Co-suppression is also observed in plants (Smyth D R., Curr. Biol., 1997, 7, R793., Martienssen, R., Curr. Biol., 1996, 6, 810). For example, if one wishes to obtain a plant in which a DNA encoding proteins that control plant cell wall component biosynthesis and wood fiber cell morphogenesis is co-suppressed, the plant in question can be transformed with a vector DNA designed to express the DNA encoding the protein, or a DNA comprising a similar sequence. Genes for use in co-suppression do not need to be completely identical to a target gene, but should comprise sequence identity of at least 70% or above, preferably 80% or above, more preferably 90% or above, and most preferably 95% or above. Sequence identity may be determined by an above-described method.

Moreover, suppression of the expression of an endogenous gene in the present invention can also be achieved by transforming a plant with a gene that encodes a protein having a dominant native trait against a protein that encodes a target gene. A “gene that encodes a protein having a dominant native trait” refers to a gene having a function that eliminates or decreases the activity of an endogenous wild type protein inherently possessed by a plant, by causing expression of the gene.

In addition, the present invention provides recombinant vectors comprising the aforementioned DNA. There are no particular limitations on the vectors of the present invention provided they comprise a promoter sequence that is transcribable in plant cells and a terminator sequence comprising a polyadenylation site required for stabilizing the transcription product. Examples include vectors that can be amplified in E. coli such as a pUC derivative, and shuttle vectors such as pBI101 (Clontech) that can be amplified in both E. coli and Agrobacterium. In addition, plant viruses such as the cauliflower mosaic virus can also be used as a vector.

A vector of the present invention can be obtained by coupling or inserting a promoter DNA, for constant or inductive expression of a promoter DNA of the present invention or a desired gene at a predetermined site of a vector. Furthermore, the promoter is inserted into the vector according to methods normally used for inserting genes into vectors. An expression vector for gene expression can be obtained by functionally connecting a desired gene to a promoter of this recombinant vector.

Promoters for constant expression are exemplified by the 35S promoter of cauliflower mosaic virus (Odell et al., Nature, 1985, 313, 810), the actin promoter of rice (Zhang et al., Plant Cell, 1991, 3, 1155), the ubiquitin promoter of corn (Cornejo et al., Plant Mol. Biol., 1993, 23, 567), etc. Furthermore, promoters for inductive expression are exemplified by promoters that are expressed by extrinsic factors such as infection and invasion of filamentous fungi, bacteria, and viruses, low temperature, high temperature, drought, ultraviolet irradiation, spraying of particular compounds, and the like. Such promoters are exemplified by the chitinase gene promoter of rice (Xu et al., Plant Mol. Biol., 1996, 30, 387.) and tobacco PR protein gene promoter (Ohshima et al., Plant Cell, 1990, 2, 95.) expressed by the infection and invasion of filamentous fungi, bacteria and viruses, the “lip 19” gene promoter of rice induced by low temperature (Aguan et al., Mol. Gen Genet., 1993, 240, 1.), “hsp 80” and “hsp 72” gene promotors of rice induced by high temperature (Van Breusegem et al., Planta, 1994, 193, 57.), “rab 16” gene promoter of Arabidopsis thaliana induced by dryness (Nundy et al., Proc. Natl. Acad. Sci. USA, 1990, 87, 1406), chalcone synthase gene promoter of parsley induced by ultraviolet irradiation (Schulze-Lefert et al., EMBO J., 1989, 8, 651.), alcohol dehydrogenase gene promoter of corn induced by anaerobic conditions (Walker et al., Proc. Natl. Acad. Sci. USA, 1987, 84, 6624) and so on. In addition, the chitinase gene promoter of rice and PR protein gene promoter of tobacco are induced also by specific compounds such as salicylic acid, and such, and the “rab 16” gene promoter is induced by the spraying of abcisic acid, a phytohormone.

In addition, for efficiently selecting cells transformed by introduction of a DNA of the present invention, the aforementioned recombinant vector preferably comprises a suitable screening marker gene, or is introduced into the cells together with a plasmid vector comprising a screening marker gene. Examples of screening marker genes used for this purpose include hygromycin phosphotransferase gene, which is resistant to the antibiotic hygromycin; neomycin phosphotransferase gene, which is resistant to kanamycin or gentamicin; and acetyl transferase gene, which is resistant to the herbicide, phosphinothricin.

In addition, the present invention provides transgenic plant cells into which a vector of the present invention has been introduced. There are no particular limitations on the cells into which a vector of the present invention is introduced, examples of which include the cells of rice, corn, wheat, barley, rye, potato, tobacco, sugar beet, sugar cane, rapeseed, soybean, sunflower, cotton, orange, grape, peach, pear, apple, tomato, Chinese cabbage, cabbage, radish, carrot, squash, cucumber, melon, parsley, orchid, chrysanthemum, lily, and saffron; however, trees such as Eucalyptus, pine, acacia, poplar, cedar, cypress, bamboo, and yew are preferable. In addition, plant cells of the present invention comprise cultured cells, as well as cells present in a plant. In addition, protoplasts, shoot primordia, multiple shoots, and hairy roots are also included.

Various techniques can be used to introduce an aforementioned expression vector into host plant cells. Examples of these techniques include transformation of plant cells by T-DNA using Agrobacterium tumefaciens or Agrobacterium rhizogenes for the transformation factor, direct introduction into a protoplast (by a method such as electroporation in which a DNA is introduced into plant cells by treating protoplasts with an electric pulse, fusion of protoplasts with liposomes and so forth, microinjection, and the use of polyethylene glycol), and the use of a particle gun.

In addition, a desired gene can be introduced into a plant, by using a plant virus as vector. An example of a plant virus that can be used is cauliflower mosaic virus. Namely, after first preparing a recombinant by inserting the virus genome into a vector derived from E. coli and so forth, the desired gene is inserted into the virus genome. Such desired genes can then be introduced into a plant by cutting out the virus genome modified in this manner from the recombinant with a restriction enzyme, and inoculating into the plant (Hohn, et al. (1982), Molecular Biology of Plant Tumors (Academic Press, New York), p. 549, U.S. Pat. No. 4,407,956). The technique for introducing a vector into plant cells or a plant is not limited to these, and includes other possibilities as well.

There are no limitations on the required vector in the case of direct insertion into a protoplast. For example, a simple plasmid such as a pUC derivative can be used. Other DNA sequences may be required depending on the method used to introduce the desired gene into plant cells. For example, in the case of using a Ti or Ri plasmid to transform plant cells, at least the sequence on the right end, and typically the sequences on both ends, of the T-DNA region of Ti and Ri plasmids must be connected so as to become an adjacent region of the gene to be introduced.

When using an Agrobacterium species for transformation, a gene to be introduced needs to be cloned into a special plasmid, namely an intermediate vector or a binary vector. Intermediate vectors are not replicated in Agrobacterium species. Intermediate vectors are transferred into Agrobacterium species by helper plasmids or electroporation. Since intermediate vectors have a region that is homologous with the T-DNA sequence, they are incorporated within the Ti or Ri plasmid of Agrobacterium species by homologous recombination. It is necessary for the Agrobacterium species used for the host to comprise a vir region. Normally, Ti or Ri plasmids comprise a vir region, and due to its function, T-DNA can be transferred into plant cells.

On the other hand, since a binary vector can be replicated and maintained in Agrobacterium species, when a vector is incorporated into Agrobacterium species by a helper plasmid or electroporation, the T-DNA of the binary vector can be transferred into plant cells due to the action of the vir region of the host.

Furthermore, intermediate vectors or binary vectors obtained in this manner, as well as microorganisms such as E. coli and Agrobacterium species that comprise them are also included in the present invention.

In addition, the present invention provides transgenic plants that have been redifferentiated from the aforementioned transgenic plant cells, transgenic plants that are progenies or clones of the transgenic plants, and breeding material of the transgenic plants. Such is a useful transgenic plant in which cell wall components and cell morphogenesis have been altered. There are no particular limitations on the alteration of cell wall components in the present invention, and include various quantitative and qualitative changes to create plants high in cellulose, low in lignin, having thick cell walls, thin cell walls, long and short fiber lengths, etc. In addition, examples of cell morphology alterations include, but are not limited to, changes in cell elongation and cell size (quantitative changes in volume).

A transgenic plant of the present invention is useful as a plant having a novel value such as increased plant growth as a result of increasing cell wall biosynthesis, altered fiber cell morphology, or increased amounts of useful components in agricultural crops. In addition, it is also useful as a plant having a novel value in developing new materials by controlling cell wall biosynthesis, increasing the digestion and absorption efficiencies of feed crops, changing fiber cell morphology, etc.

In the present invention, a “transgenic plant” refers to a plant having the aforementioned transgenic plant cells, and includes, for example, a transgenic plant regenerated from the aforementioned transgenic cells. Although the methods used to regenerate individual plants from transformed plant cells vary according to the type of plant cell, an example of a method used in rice plants is the method of Fujimura et al. (Fujimura et al., Plant Tissue Culture Lett., 2, 74, 1995), the method of Shillito et al. (Shillito et al., Bio/Technology, 7, 581, 1989) in corn plants, the method of Visser et al. (Visser et al., Theor. Appl. Genet., 78, 589, 1989) in potato plants, the method of Akama et al. (Akama et al., Plant Cell Rep., 12, 7, 1992) in Arabidopsis thaliana, and the method of Doi et al. (Japanese Patent Application No. Hei 11-127025) in Eucalyptus plants. Transgenic plants produced according to these methods or transgenic plants obtained from their breeding materials (such as seeds, tubers, or cuttings) are included in the present invention.

The present invention includes a process of producing a plant from a plant seed by introducing into a host a gene specifically expressed by a plant (particularly a tree) during cell wall formation and/or specifically expressed during cellulose biosynthesis, a homolog thereof, or an expression vector comprising a promoter region that is contiguous with these genes to obtain transgenic cells, regenerating a transgenic plant from said transgenic cells, and obtaining a plant seed from the resulting transgenic plant.

A process of obtaining a plant seed from a transgenic plant refers to a process in which, for example, a transgenic plant is acquired from a rooting medium, replanted in a pot containing moist soil, and grown at a constant temperature to form flowers, and finally seeds. In addition, a process of producing a plant from a seed refers to a process in which, for example, once a seed formed in a transgenic plant has matured, the seed is isolated, sowed on moist soil, and then grown at a constant temperature and luminosity, to produce a plant.

The exogenously introduced DNA or nucleic acid in a transformed plant can be confirmed by known methods, such as PCR or Southern hybridization, or by analyzing the nucleotide sequence of the plant's nucleic acid. To extract DNA or nucleic acid from a transformed plant, the known method of J. Sambrook et al. may be used (Molecular Cloning, 2^{nd} edition, Cold Spring Harbor laboratory Press, 1989).

To conduct PCR analysis of a DNA of the present invention that exists in a plant, an amplification reaction is carried out using, as a template, nucleic acid extracted from the regenerated plant by the above-mentioned method. Amplification reaction may be carried out in a reaction mixture containing, as primers, synthesized oligonucleotides comprising nucleotide sequences appropriately selected according to the nucleotide sequence of a DNA of the present invention. An amplified DNA fragment comprising a DNA sequence of the present invention may be obtained by repeating several dozen cycles of the denaturation, annealing, and extension steps of the DNA amplification reaction. The respective amplified DNA fragments can be separated by, for example, electrophoresing the reaction solution containing the amplified products on agarose gel. DNA fragments corresponding to a DNA of the present invention can then be confirmed.

Having obtained a transformed plant in which a DNA of the present invention has been inserted into the chromosomes, one can obtain the plant's offspring by sexual or non-sexual reproduction. Also, it is possible to mass-produce such plants by obtaining reproductive materials (such as seeds, fruits, cuttings, stem tubers, root tubers, shoots, calluses, and protoplasts) from the above plant, or its offspring or clones.

A stable supply of biomass, mainly cellulose, can be provided by cultivating a transgenic plant of the present invention on a larger scale using clone planting. At present, fossil resources are used in large amounts in industrial productions as raw materials and fuel (energy). With respect to alternative energy in particular, although the direct combustion of wood biomass (for fuel) is routinely carried out in developing countries, a more effective approach would be possible by converting the biomass into a more user-friendly form (such as alcohol, and specifically ethyl alcohol). In reality, ethanol is produced in Brazil and other countries, by alcohol fermentation of waste syrup obtained from squeezed sugar cane, and is used as an automobile fuel. In the U.S. and EU as well, there are a growing number of examples of alcohol fermentation after initially hydrolyzing starch from sweet potatoes and corn into glucose. In August 1999, the U.S. announced that, “the rate of biomass energy utilization will be increased to 10% of all primary energy by the year 2010”. One of the objectives is to use gasoline mixed with ethanol refined from biomass. A specific example is “gasohol” (a 10% blend of ethanol in gasoline) made from corn. Gasohol is used in 20 states, mainly by those in the corn belt, and currently accounts for about 1% of all automobile fuel used in the U.S. Gasohol accounts for 40% of the gasoline share in certain states where sugar cane is cultivated. All U.S. automobile manufacturers have certified the use of gasohol as fuel, and more specifically, General Motors Corporation and DaimlerChrysler Corporationrecommend its use. In the EU, a project is underway to increase the share of recyclable energy to 12% of all energy, with the aim of reducing levels of greenhouse gases to 8% of the level of 1990 by the year 2010. This project has set the goal of substituting biofuel (fuel derived from biological resources) for 5% of all fossil fuels by 2005, as an alternative automobile fuel. The EU's energy utilization plan calls for the use of solar cells (degree of contribution: 1%), wind power (19%), and biomass-cogeneration (80%), thus indicating the considerable expectations being placed on biomass. In addition, cultivation of biocrops for energy utilization is expected to account for the largest land utilization area by 2015. On the other hand, from the viewpoint of increasing food production, large-scale consumption of grains and potatoes as industrial raw materials would be limited in the future. Thus, if it were possible, for example, to grow large numbers of the present invention's Eucalyptus trees having a high cellulose content, it would be possible to obtain glucose by hydrolysis or enzyme degradation (cellulase) using the resulting lignocellulose as raw material, and in turn enable large-scale production of ethanol by alcohol fermentation. Basic technology for such processes has already been established. Moreover, the technology for producing biodegradable plastics (polylactic acid) using glucose as raw material is already established, and practical applications using potato starch is progressing on an of industrial production scale. In the future however, it is predicted that biomass from trees will become the mainstream replacing grains, which is a food item. Furthermore, although it will be necessary to overcome technical problems in the future to achieve practical application of lignin, applications in plastics and adhesives are expected. In addition, from the viewpoint of energy, although lignin is contained in waste liquid (referred to as black liquor) following its chemical decomposition in the production of pulp in the paper industry, it is being used as factory fuel after extracting the required chemicals from the waste liquid. In other words, a portion of fuel is already dependent on wood biomass.

In addition to conventional use as raw materials, there is also a considerable potential for creating an alternative energy to petroleum through biomass conversion, as well as the development of new plastics from cellulose and hemicellulose (both being technically possible), as a result of stable and large-scale cultivation of wood biomass and the recycling of that wood biomass through afforestation as in the present invention. Moreover, the spread of wood biomass will contribute to solving energy security problems and environmental issues, while simultaneously leading to the development of new industries, including agricultural forestry, and the creation of employment opportunities.

### BRIEF DESCRIPTION OF THE DRAWINGS

FIG. 1 shows the expression intensities of major gene clusters in Eucalyptus reaction wood. Two types of mRNA extracted from Eucalyptus reaction wood and normal wood were each labeled with two types of fluorochromes (cy3, cy5); hybridization was carried out using these mRNAs as probes in an oligo microarray analysis. The images resulting from scanning fluorescent intensity were analyzed with analytical software (Luminator Ver. 1.0, Rosetta). All repeated experiments were integrated to a statistical reliability of 99.9%, and the relative expression intensities of the major gene clusters in Eucalyptus reaction wood were graphed. In the photo, +(red) indicates genes for which a significant increase in expression was observed, +(green) indicates genes for which a significant decrease in expression was observed, while+(blue) indicates genes for which changes in expression were not observed.

### BEST MODE FOR CARRYING OUT THE INVENTION

Although the following examples provide a more detailed explanation of the present invention, the present invention is not limited thereto. The experimental procedures were carried out in accordance with “Cloning and Sequencing” (Watanabe, I., Sugiura, M., ed., Norin-Bunka Publishing (1989)) and “Molecular Cloning” (Sambrook, et al. (1989), Cold Spring Harbor Laboratory Press) unless indicated otherwise.

### EXAMPLE 1

### Production of a Eucalyptus EST Database

(1) Extraction of RNA from Eucalyptus

The thickly grown part of the trunk (secondary wall hypertrophic band; tissue rich in cambium), leaves, and roots, were selected as Eucalyptus tissue for extraction, envisioning gene expression by various circumstances such as tension stress and stress due to exposure to salt solutions. The method described in Hiono, et al. (Japanese Patent Application No. Hei 6-219187) was used as the basic extraction procedure. As an example of this method, the following provides a detailed explanation of the RNA extraction method using Eucalyptus root obtained by a hydroponic cultivation.

Young Eucalyptus (Eucalyptus camaldulensis) plants grown for two months were transferred to a hydroponics tank. Hydroponic cultivation was carried out using the culture medium of Hoagland-Amon, et al. The composition of the hydroponic culture medium was: 5.0 mM KNO₃, 3.0 mM Ca(NO₃)₂, 2.0 mM NH₄H₂PO₄, 2.0 mM MgSO₄, 47 μM H₃BO₃, 9.0 μM MnCl₂, 36 μM FeSO₄, 3.1 μM ZnSO₄, 0.16 μM CuSO₄ and 75 μM (NH₄)₆Mo₇O₂₄. This medium was prepared using desalinated water, and the pH was adjusted to 6.0 daily with 0.1 M NaOH or KOH. Moreover, the whole culture medium was replaced once a week. When conducting stress treatment, a culture medium to which NaCl was sequentially added to a final concentration of 50, 100, 200, and 300 mM from day 1 to day 4 was used for the stress treatment group, while a culture medium to which NaCl was not added was used for the control group. Ten grams of the root were cut into small pieces and homogenized in liquid nitrogen on day 4. This was then transferred to a 50 ml centrifuge tube (NUNC), and homogenized for 5 minutes with a homogenizer after adding 10 g of glass beads. Solvent extraction of the homogenized sample was repeated (about three times), until the supernatant was colorless using a methanol solution comprising dithiothreitol (1 mg/ml). Following completion of extraction, the sample was freeze-dried. The freeze-dried sample was mixed with 25 ml of pH 9 100 mM CHES buffer (to which 20 mg of dithiothreitol and 10 mM vanadyl ribonucleoside compound solution were added immediately prior to use) and incubated for 30 minutes at 65° C. After incubation, 5 M aqueous sodium chloride solution and 10% CTAB solution were added to the sample solution, so as to make the sodium chloride concentration 1.4 M, and the CTAB concentration 1% (w/v). After mixing the sample solution well and incubating for 10 minutes at 65° C., an equal volume of chloroform: isoamyl alcohol (24:1) solution was added and this was gently but thoroughly mixed. After mixing, the supernatant was recovered by centrifugation. 55% by volume of isopropanol was added to the supernatant followed by cooling with ice for 1 hour. A precipitate was obtained by centrifugation, and phenol extraction was carried out after dissolving the precipitate in water. 10% by volume of 3 M sodium acetate and 60% by volume of isopropanol were added to the supernatant following phenol extraction, and after mixing well, the precipitate was recovered by centrifugation. After dissolving the precipitate in sterile water, 12 M lithium chloride solution was added to a final concentration of 3 M, and after mixing well, the solution was cooled with ice for 1 hour. An RNA precipitate was recovered by centrifugation, and after washing and drying, the RNA was finally dissolved in 100 μL of water to obtain a total RNA fraction. As a result, 610 μg each of total RNA were obtained from the roots of the stress treatment group and control group. mRNA was purified from the total RNA fraction using the PolyATract mRNA Isolation System III & IV Kits (Cat. Nos. Z5300 and Z5310, Promega, USA). As a result, from the 610 μg of total RNAs, 1.3 μg of mRNA were obtained from the sample of the stress treatment group and 1.8 μg of mRNA were obtained from the control sample.

(2) Construction of cDNA Library

cDNA was synthesized using the Smart cDNA Library Construction Kit (Clontech) from Eucalyptus mRNA derived from each of the tissues and circumstances according to the method described in (1) above, to ultimately construct a phagemid library. The genomic DNA library produced in this manner comprised independent clones of 1×10⁶ pfu or more each. Furthermore, library amplification was carried out on a portion of the constructed library. Clone analysis was done without amplifying.

(3) Deciphering cDNA Clones and Database Construction

Clones were randomly selected from the Eucalyptus phagemid cDNA library derived from each tissue, and after purifying the plasmids, an enzyme reaction was carried out using the Dye Terminator Sequence Kit (Amersham) followed by acquisition of nucleotide sequence data using a large-scale, high-speed sequencer (Amersham).

The data was analyzed using analytical software after deleting known plasmid sequences, and homologous sequences were extracted by a clustering procedure. Subsequently, a comparative search was done using the entire database of GenBank, U.S.A, one of the genetic information databases, to roughly predict (annotation) the function.

The size of the final database is shown in Table 1.

TABLE 1

| | | Total number | | |
|---|---|---|---|---|
| | | of nucleotide | Number of | Total |
| | Subject | sequences | clusters (total | number of |
| No. | tissue | deciphered | constituent no.) | singlets |
| OJI001, | Trunk | 20645 | 2762 (16026) | 4619 |
| OJI005 | | | | |
| OJ1004 | Leaf | 10171 | 1021 (7574) | 2597 |
| OJI002, | Root | 10726 | 1511 (7169) | 3557 |
| OJI003 | | | | |
| OJI001-005 | All tissue | 41542 | 4660 (34206) | 7336 |

### EXAMPLE 2

### Extraction of Genes Specifically Expressed in Eucalyptus Reaction Wood Tissue

(1) Production of a Eucalyptus Trunk-Specific Oligo Microarray

A Eucalyptus oligo microarray was produced targeting the entire sequence excluding the overlapping sequences from 0JI001 and 0JI005 according to the Eucalyptus EST database shown in Table 1. Actual production of the microarray was commissioned to Agilent Technologies, Inc. (Japanese representative: Yokogawa Analytical Systems Inc.). Details are described in the following web site: http://www.agilent.com/cag/country/JP/products/PCol494.html.

The Eucalyptus oligo microarray produced in this manner comprised 8400 oligo DNA, and was able to cover a majority of the genes recognized to be expressed in the Eucalyptus trunk.

(2) Extraction of Genes Specifically Expressed in Eucalyptus Reaction Wood Forming Tissue by Microarray Analysis

The biosynthesis of cellulose (a major component of the cell wall) in broad-leaved trees in particular, is known to involve the formation of tissue whose cell wall cellulose content roughly doubles as a result of the external tension stress. Comprehensive determination of the series of genes responsible for cellulose biosynthesis in particular, during cell wall formation, is possible by analyzing this tissue using the aforementioned genomic analysis. In addition, differences in expression of each component gene can also be determined by combining gene expression analysis by microarray analysis using EST data.

Tension wood has long been known as a characteristic phenomenon that results from the aforementioned external mechanical stress. Reaction wood of broad-leaved trees refers to the special secondary wood that is formed as a result of a tree trunk having detected a change in the direction of gravity, when responding to an external tension stress. Compared with ordinary wood, a cellulose increase and lignin and hemicellulose decrease is seen in cell wall components. In addition, morphological observations reveal that xylem distribution density decrease to half of that of ordinary wood, and that the leaning angle of microfibrils in the cell walls became nearly parallel with the axial direction of cell growth. Moreover, fiber length is observed to increase by roughly 20%. Although the details are unknown, it is thought that a growth strain results, by which the trunk, at locations where longitudinal growth is already over, attempts to return to the correct position in response to the leaning. As the leaning persists, a single branch finally begins new vertical elongation, in place of the main trunk. However, during the time the trunk attempts to rise upward in response to the leaning, tissue is formed in which cellulose content becomes extremely high in the tissue at the top of the leaned trunk. When a cross-section of the trunk is observed, semi-transparent tissue that is different from ordinary wood tissue can also be visually observed easily. Although general descriptions on reaction wood are always disclosed in technical literature relating to wood, a paper by Baba, et al. (Mokuzai Gakkaishi (Academic Journal of Wood and Lumber) 42, 795-798, 1996) has detailed data on the chemical and histological properties of reaction wood as related to Eucalyptus.

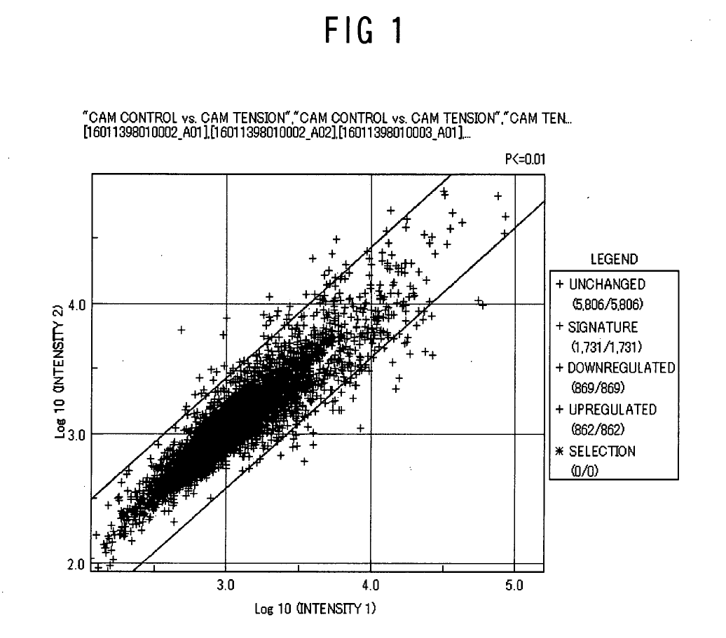

The present inventor extracted total RNA from a cloned line of Eucalyptus camaldulensis (CPT1), in accordance with the RNA extraction method described in Example 1, using normal wood and reaction wood. A specific reaction wood tissue is formed in the upper portion of the trunk, by artificially pulling and tilting an ordinary growing trunk to an angle of about 45 degrees. mRNA was purified from the total RNA obtained from ordinary wood and reaction wood, using the PolyATract mRNA Isolation System (Promega). The two types of mRNA obtained in this manner were then each labeled with two types of fluorochromes (cy3, cy5) followed by hybridization using them as probes in oligo microarray analysis (FIG. 1). The hybridization method, including labeling, was performed in accordance with the analysis protocol as directed by Agilent Technologies, Inc.

As a result, genes were broadly classified into a gene cluster with a predominantly high expression, a gene cluster with a low expression, and a gene cluster with a virtually unchanged expression, in Eucalyptus reaction wood as compared to ordinary trunks. In particular, the gene cluster that demonstrates a predominantly high expression, is thought to be involved in cell wall component biosynthesis and wood fiber cell morphogenesis. More specifically, this gene cluster is thought to be directly involved in the expression of traits such as high cellulose content and low lignin content that are characteristic to reaction wood.

### INDUSTRIAL APPLICABILITY

Use of DNA of the present invention enabled the artificial control of wood biomass production in trees. Particularly it was possible to change the quantity and quality of the essence of wood mass-the cell wall components (cellulose, hemicellulose, and lignin). Furthermore, fiber morphology (wood fiber cell elongation) could be freely altered. Namely, quantitative increases and qualitative modifications of essential wood biomass, and the resultant expansion of uses in future energies and in applications as industrial raw materials are expected, hopefully replacing the current fossil materials in various fields.

1. A DNA whose expression increases during plant cell wall biosynthesis and wood fiber cell morphogenesis, wherein the DNA is described in (a) or (b) below:
(a) a DNA that hybridizes under stringent conditions with a DNA comprising a nucleotide sequence described in any one of SEQ ID NOs: 1 to 862; or,
(b) a DNA encoding a protein having 50% or more homology with a protein comprising an amino acid sequence encoded by the DNA of (a).

2. The DNA of claim 1, wherein expression increases in plant reaction wood forming tissue.

3. A DNA whose expression decreases during plant cell wall biosynthesis and wood fiber cell morphogenesis, wherein the DNA is described in (a) or (b) below:
(a) a DNA that hybridizes under stringent conditions with a DNA comprising a nucleotide sequence described in any one of SEQ ID NOs: 863 to 1731; or,
(b) a DNA encoding a protein having 50% or more homology with a protein comprising an amino acid sequence encoded by the DNA of (a).

4. The DNA of claim 3, wherein expression decreases in plant reaction wood forming tissue.

5. The DNA of claim 1 or 3, wherein the plant is Eucalyptus.

6. A DNA encoding a protein comprising an amino acid sequence in which one or more amino acids are substituted, deleted, added and/or inserted in an amino acid sequence encoded by the DNA of claim 1 or 3.

7. A promoter DNA of the DNA of claim 1 or 3.

8. A DNA described in any one of (a) to (e) below:
(a) a DNA encoding an antisense RNA complementary to a transcription product of the DNA of claim 1 or 3;
(b) a DNA encoding an RNA having ribozyme activity that specifically cleaves a transcription product of the DNA of claim 1 or 3;
(c) a DNA encoding an RNA that suppresses expression of the DNA of claim 1 or 3 by RNAi effects;
(d) a DNA encoding an RNA that suppresses expression of the DNA of claim 1 or 3 by co-suppression effects; and,
(e) a DNA encoding a protein having a dominant negative trait against a transcription product of the DNA of claim 1 or 3.

9. A recombinant vector comprising the DNA of claim 1 or 3.

10. A microorganism retaining a plasmid comprising the vector of claim 9.

11. A transgenic plant cell introduced with the vector of claim 9.

12. A transgenic plant that is re-differentiated from the transgenic plant cell of claim 11.

13. A transgenic plant that is a progeny or a clone of the transgenic plant of claim 12.

14. A breeding material of the transgenic plant of claim 12.

15. A breeding material of the transgenic plant of claim 13.

16. A recombinant vector comprising the DNA of claim 8.

17. A microorganism retaining a plasmid comprising the vector of claim 16.

18. A transgenic plant cell introduced with the vector of claim 16.

19. A transgenic plant that is re-differentiated from the transgenic plant cell of claim 18.

20. A transgenic plant that is a progeny or a clone of the transgenic plant of claim 19.

21. A breeding material of the transgenic plant of claim 19.

22. A breeding material of the transgenic plant of claim 20.

23. A microorganism retaining a plasmid comprising the promoter DNA of claim 7.


SEQUENCE LISTING (1731 sequences)

SEQ ID NO: 1 - DNA, 60 bp, Eucalyptus camaldulensis
tcttcctgccgggaatccaagattatagtagcctcggcgtctcttggtttgtatttgtat

SEQ ID NO: 2 - DNA, 60 bp, Eucalyptus camaldulensis
tccatgtaatagccaaaaatgatcagttgctgtatcttgagatgagtgaggattctgcat

SEQ ID NO: 3 - DNA, 60 bp, Eucalyptus camaldulensis
gctgtcttaacgatgtcgatcatttggatttcggattgaataaagaaggacgatgctcat

SEQ ID NO: 4 - DNA, 60 bp, Eucalyptus camaldulensis
agtaagaggcagtgacagtatcaccaacataattgagtcgggtcatacttgcatatgtta

SEQ ID NO: 5 - DNA, 60 bp, Eucalyptus camaldulensis
cttgacaacctttgaagggaaggatttcatggggagaccattaagagtggcacgaagtaa

SEQ ID NO: 6 - DNA, 60 bp, Eucalyptus camaldulensis
tgcgagattgtatgtagtctagtcatgctttcatggggaatccgagttgtaagatgtgtt

SEQ ID NO: 7 - DNA, 60 bp, Eucalyptus camaldulensis
tgttgcatccgaaggaatcgcttggctatgcggatatctacttatctgatgttgtcagca

SEQ ID NO: 8 - DNA, 60 bp, Eucalyptus camaldulensis
gtgaattggcccactgccattatttgttctttccatttatgtccaggttcggtttgtttg

SEQ ID NO: 9 - DNA, 60 bp, Eucalyptus camaldulensis
gacgagcttcttgatctagtgttttctttctcgtgctcctttttctgctgaatgaaatac

SEQ ID NO: 10 - DNA, 60 bp, Eucalyptus camaldulensis
tctctctctctctctctctctctgctgagaaaagacaccttgctatcattagcgtattga

SEQ ID NO: 11 - DNA, 60 bp, Eucalyptus camaldulensis
tgctatgtgctgcttgtatatgtctctgctttattgctagtagggtttgttaatgaagta

SEQ ID NO: 12 - DNA, 60 bp, Eucalyptus camaldulensis
cactcccagctggtatggtgtagtttttagcctctttattttaatacagaggaagacggg

SEQ ID NO: 13 - DNA, 60 bp, Eucalyptus camaldulensis
gatgtgaatttacttgtgactgaagatttccttgtttggcagctgctgctacttgctgtc

SEQ ID NO: 14 - DNA, 60 bp, Eucalyptus camaldulensis
cttttctgttagaacgagtggagcttagtgaatcatctgttgcccatcctcgtccttcta

SEQ ID NO: 15 - DNA, 60 bp, Eucalyptus camaldulensis
tcaagatcggcggctacgacatccccaaggggtcgaacgtccacgtgaatgtatgggcca

SEQ ID NO: 16 - DNA, 60 bp, Eucalyptus camaldulensis
agcacgactggttcttcgctcttgttcagtattatcataaaaattctgactatagcccaa

SEQ ID NO: 17 - DNA, 60 bp, Eucalyptus camaldulensis
ctcccaaggtctcagcacactttggatgtatttttattagctggctacttgactcttaat

SEQ ID NO: 18 - DNA, 60 bp, Eucalyptus camaldulensis
aggaccaagttcttaagcagtcttgtgtaatttctgcttcatgtgatcccgaggccactt

SEQ ID NO: 19 - DNA, 60 bp, Eucalyptus camaldulensis
aggattgggaaagagaatgaaacatgtgcccctccttttgctatttcccctcaccatcaa

SEQ ID NO: 20 - DNA, 60 bp, Eucalyptus camaldulensis
tttctgctgatgtttgctaattttctgcgacgacttcaccagggatgtgcgcttgtatct

SEQ ID NO: 21 - DNA, 60 bp, Eucalyptus camaldulensis
ccgtgtggtacggacgtgtttgtcgtatcgcaagggattagatgcaatcaaatactgtaa

SEQ ID NO: 22 - DNA, 60 bp, Eucalyptus camaldulensis
ttaccgcggacaccacgtctttctaaatacgagtgcttcggtgcagttcttcacaaaaaa

SEQ ID NO: 23 - DNA, 60 bp, Eucalyptus camaldulensis
aacgggagatttatattgggagtttattatgagcttcgtgccacgtcttgtatgatgata

SEQ ID NO: 24 - DNA, 60 bp, Eucalyptus camaldulensis
aacgcatggctatcagaagagctcgactcaacaagccaagagaggctgaagtgggtgcaa

SEQ ID NO: 25 - DNA, 60 bp, Eucalyptus camaldulensis
ctgtactgtaacacgagagatcgtgtcacgcaactctttcacctgatgtttctactccaa

SEQ ID NO: 26 - DNA, 60 bp, Eucalyptus camaldulensis
tttttgctgaatttgaagatttgtactgctctctgagacatatctcctgggtttccaaat

SEQ ID NO: 27 - DNA, 60 bp, Eucalyptus camaldulensis
aagtcattctcgacgtgctctgtaatccttgtgattgtgactgttctcctgatcagtggt

SEQ ID NO: 28 - DNA, 60 bp, Eucalyptus camaldulensis
ttcttgctaagtggtttggatgttctgcctatttaataggcttatgttccgatgaataaa

SEQ ID NO: 29 - DNA, 60 bp, Eucalyptus camaldulensis
atgaaaggcgaaagcagaaggtattttcggaagccaatcttttggggcgtgccattagat

SEQ ID NO: 30 - DNA, 60 bp, Eucalyptus camaldulensis
tcttttactttgctgagaagagcgtttgcacacactgtatttgtcgtctgcgggttaaaa

SEQ ID NO: 31 - DNA, 60 bp, Eucalyptus camaldulensis
atttctgctgcgaatcctgtgctttaacccaggagtaccgcgagctcgaaaggcgcggat

SEQ ID NO: 32 - DNA, 60 bp, Eucalyptus camaldulensis
ttgcgaatgaggagaagaaagaatgtcatggtgaatcaagcgacaaacttgaggagcatg

SEQ ID NO: 33 - DNA, 60 bp, Eucalyptus camaldulensis
ggcatgggaatccccaggtgttctactatgcagagtgtatgtccttgtggatcttaatat

SEQ ID NO: 34 - DNA, 60 bp, Eucalyptus camaldulensis
tactttaatttgtggccttattttcgcagaattccctgggactgcgtgtttaaaaagatg

SEQ ID NO: 35 - DNA, 60 bp, Eucalyptus camaldulensis
cggtttagagatctaatgtggatatttatcgtgatacattgagggttattacttagcatc

SEQ ID NO: 36 - DNA, 60 bp, Eucalyptus camaldulensis
cctccctcctcctctctcctagaaaatattgtgtgtacctaagaggatggaaagtgagta

SEQ ID NO: 37 - DNA, 60 bp, Eucalyptus camaldulensis
ccattatggccgggtgagatatgaataatgaaatttgctatttctcctagttaaaaaaaa

SEQ ID NO: 38 - DNA, 60 bp, Eucalyptus camaldulensis
tacatgtaactcaattttgcccttttgggtgcctagagcaatggccgaatttctttcccc

SEQ ID NO: 39 - DNA, 60 bp, Eucalyptus camaldulensis
tttcaagtgtaccgatccaccaaactttgtttaaagtttcctggaccttttgaaaaatca

SEQ ID NO: 40 - DNA, 60 bp, Eucalyptus camaldulensis
tcattcaatagtcatgaaggtatgactgcagagaattgaaaagaagtaaagaagctgttc

SEQ ID NO: 41 - DNA, 60 bp, Eucalyptus camaldulensis
tacttgtggcagaaaatgtagggtatgcctgaatgctgggattcaacaggcagatgtgtt

SEQ ID NO: 42 - DNA, 60 bp, Eucalyptus camaldulensis
atcatttcttgtatttcgagactgcgtttcggaaggactttgcaaagccctttggacctc

SEQ ID NO: 43 - DNA, 60 bp, Eucalyptus camaldulensis
gtatggtgactagtaactaggaactgcataaatccatgttaatcttgtcatgttcaagcg

SEQ ID NO: 44 - DNA, 60 bp, Eucalyptus camaldulensis
gaccgcgacttgtccggtgaccatcgtcaaggagccgacttctcatggtttctgatcgat

SEQ ID NO: 45 - DNA, 60 bp, Eucalyptus camaldulensis
gtggtagaaaaatgcttctcagatgcttaccgtgagatgacaaagttgtatgcaagaaca

SEQ ID NO: 46 - DNA, 60 bp, Eucalyptus camaldulensis
cctttctgggtgaacataatgaacagaaggatcgatttcccgataagcgacggagataat

SEQ ID NO: 47 - DNA, 60 bp, Eucalyptus camaldulensis
tacattaactggttggagtcttttcttggggtcagcattttgtctttctggacctccatg

SEQ ID NO: 48 - DNA, 60 bp, Eucalyptus camaldulensis
tgtcaccattctgaactggtccttcgtaagaaacgatcagccgagatttgagacttgcta

SEQ ID NO: 49 - DNA, 60 bp, Eucalyptus camaldulensis
agggattgaaacttttcagagggctctgccgaatggcaaggttaagcagatcttcttctt

SEQ ID NO: 50 - DNA, 60 bp, Eucalyptus camaldulensis
gagattccacttcagttgtgttccaaaatgttaaaccctcgggtgttgcatttcagcaca

SEQ ID NO: 51 - DNA, 60 bp, Eucalyptus camaldulensis
tggatgtacatgagacgtgctcatggtttaccaagtcaataatatgtttgattaattgga

SEQ ID NO: 52 - DNA, 60 bp, Eucalyptus camaldulensis
gatgtttcatggtgaggtattcctgtaagtgtgttcctttcggacgagtggtgtaatgca

SEQ ID NO: 53 - DNA, 60 bp, Eucalyptus camaldulensis
gtatatatgagtatgaccgcgtgatattctggaaaaaatgtttgcattgtgttcaaaacg

SEQ ID NO: 54 - DNA, 60 bp, Eucalyptus camaldulensis
ttttggacttccagattgttggctttgctgattcatatgataaacggttgctcatttttg

SEQ ID NO: 55 - DNA, 60 bp, Eucalyptus camaldulensis
tggtgctgtcgaattggtgcaattttaacaaattcagagccattatctcgctgttgcatt

SEQ ID NO: 56 - DNA, 60 bp, Eucalyptus camaldulensis
gtcagtcatgtacttgagtacttgagctagcaagtatcctaatgaatttcgaattccgca

SEQ ID NO: 57 - DNA, 60 bp, Eucalyptus camaldulensis
agtttgcattgatattgaaagaacgtgatgaagaatttagtatgtggacctgcacgactt

SEQ ID NO: 58 - DNA, 60 bp, Eucalyptus camaldulensis
gcattgagatcatgtacattctttgtaaagggaaagaaaaggaaatctcctcgacctttt

SEQ ID NO: 59 - DNA, 60 bp, Eucalyptus camaldulensis
tgtacttgtagcagcttcatcggttgtaacatatggtttgtctttaaaggaaatagacca

SEQ ID NO: 60 - DNA, 60 bp, Eucalyptus camaldulensis
ctggcccttccaggtctaaaaagaagttgggggatccgatctgctccctgccctcattca

SEQ ID NO: 61 - DNA, 60 bp, Eucalyptus camaldulensis
agaagacagaaggatgccccgatgctgtgtcgagggaagtcactgcggaagaggatctca

SEQ ID NO: 62 - DNA, 60 bp, Eucalyptus camaldulensis
tgtttgctccttcccttggagtggtttgtcaatgaatgaatggaatgaaccatcctctcg

SEQ ID NO: 63 - DNA, 60 bp, Eucalyptus camaldulensis
agcaagcaagcaagcaagcaagcaagagcaagcgacgtaattcccgaatgtgaaaaatca

SEQ ID NO: 64 - DNA, 60 bp, Eucalyptus camaldulensis
ctctagtcttaccttccgctccagactctcaacttcttttctttagaggacagaagagca

SEQ ID NO: 65 - DNA, 60 bp, Eucalyptus camaldulensis
ggggagagaattgtgcagtgtagtgtcttcaataattcctagatgccaatgtactgtcat

SEQ ID NO: 66 - DNA, 60 bp, Eucalyptus camaldulensis
tcacggtgtgggcaatgtcgacaaccaactctggaaagaaagggttacgtgatattctat

SEQ ID NO: 67 - DNA, 60 bp, Eucalyptus camaldulensis
gcatgcaggtccgttgatgtgtcaagcgtgtctaatccgacgcataagcaagctctataa

SEQ ID NO: 68 - DNA, 60 bp, Eucalyptus camaldulensis
ccatccatggaagctggtactgcatctgtatctttggcattgcaatatctctctcctaca

SEQ ID NO: 69 - DNA, 60 bp, Eucalyptus camaldulensis
gcaacaaggaatctatcgaaacatgtagctctggaattgcaatactaagaactgctctct

SEQ ID NO: 70 - DNA, 60 bp, Eucalyptus camaldulensis
taaagggtaagaccatggaccgtggaaatactggttaaattctcaaatatcatccggtgg

SEQ ID NO: 71 - DNA, 60 bp, Eucalyptus camaldulensis
aatcgtcacgctcaacagttctaggacagttcaacgtacagtcaccaatattgccggaaa

SEQ ID NO: 72 - DNA, 60 bp, Eucalyptus camaldulensis
gagtggagctccatttgctcgtaagtttgagaaggacgaccctgttcttaataggattga

SEQ ID NO: 73 - DNA, 60 bp, Eucalyptus camaldulensis
ggtcataacgaaagtgtccatttaattctgattattgagatggagggggtctgtatgttt

SEQ ID NO: 74 - DNA, 60 bp, Eucalyptus camaldulensis
gtgtgtgtaaattggtggtgttatgtaatgatgatttacgctgctggttgcgtatgcggt

SEQ ID NO: 75 - DNA, 60 bp, Eucalyptus camaldulensis
aaataaactaggcagcggatttacgtcgcttagtcagtgggatctactccacggaattca

SEQ ID NO: 76 - DNA, 60 bp, Eucalyptus camaldulensis
gctggctcgagtggaatgccaccttcgcgctcgacacgctctactacggcgagtacatga

SEQ ID NO: 77 - DNA, 60 bp, Eucalyptus camaldulensis
gttatggactctcgattgttgagctctgaggtatataattctgctcatgtttgtacaaat

SEQ ID NO: 78 - DNA, 60 bp, Eucalyptus camaldulensis
gacagtgtcatggtacggatgtgttgcagattaatgaaggatcattctgtgttttcgtcg

SEQ ID NO: 79 - DNA, 60 bp, Eucalyptus camaldulensis
tttccctctgtttaacccttttgttcgttcagaagtatggttatagcgtctgtaagttga

SEQ ID NO: 80 - DNA, 60 bp, Eucalyptus camaldulensis
ctaggagagcggagccgatttggaagagttaatgtcaagaaagttgtaacttgcaagttg

SEQ ID NO: 81 - DNA, 60 bp, Eucalyptus camaldulensis
gttgaggagatctgttatttgcatgtgtttgttaaactagcaaaacctctctctctctct

SEQ ID NO: 82 - DNA, 60 bp, Eucalyptus camaldulensis
tgtgggaataacctgttggggatctggtattgcaatttaccacccgaagttgtaatctag

SEQ ID NO: 83 - DNA, 60 bp, Eucalyptus camaldulensis
tgagcatctgcacttttgacatggtctgcttttaggttttctacatcagaagaaaccaag

SEQ ID NO: 84 - DNA, 60 bp, Eucalyptus camaldulensis
gcttgtagctaggtccaaatgctttggactaaattttttaatttcaaaactctctctctc

SEQ ID NO: 85 - DNA, 60 bp, Eucalyptus camaldulensis
atatcgcttgtaggcctcaggaaaagttgtacaagaataagtttaatttatagtcaaggc

SEQ ID NO: 86 - DNA, 60 bp, Eucalyptus camaldulensis
aggtaaaatgcgaagggagtgcctggtgatctccatgaaaatggatatgtgttgctgtgt

SEQ ID NO: 87 - DNA, 60 bp, Eucalyptus camaldulensis
atggtgtcctctggcttgctgaggctttgcactgcttaaatgggatattttactatgcac

SEQ ID NO: 88 - DNA, 60 bp, Eucalyptus camaldulensis
ccctgtatgtcattcgcaattattcaaaatttagtgtctgccaactaggggaagaacttt

SEQ ID NO: 89 - DNA, 60 bp, Eucalyptus camaldulensis
tcattgccgtgactatacctgcgatgtttctttgaaatgttatattgtgaagctagatct

SEQ ID NO: 90 - DNA, 60 bp, Eucalyptus camaldulensis
ttcagttgatctgaagtccaggattcctgtcgtgtttatcttataaaagccaagcctttt

SEQ ID NO: 91 - DNA, 60 bp, Eucalyptus camaldulensis
cacccaaagatctaagcttcgttggctatacatacaagaactttgatgccgtgaaggggt

SEQ ID NO: 92 - DNA, 60 bp, Eucalyptus camaldulensis
tcagtgaaagcaagtgctatctgaagggcattttgtcctaatatataagggacctatgtt

SEQ ID NO: 93 - DNA, 60 bp, Eucalyptus camaldulensis
cagaagctggatctaggttatttcggtgaagggcgttagattaattctcgggtcataacc

SEQ ID NO: 94 - DNA, 60 bp, Eucalyptus camaldulensis
acaatcgatccgaatgcaaagatcgagttcaagccaaacacagaagacgacccacacaag

SEQ ID NO: 95 - DNA, 60 bp, Eucalyptus camaldulensis
attgaacataataaatcctttgtactcctttggaggtagtgcatagttgaaactcaaagg

SEQ ID NO: 96 - DNA, 60 bp, Eucalyptus camaldulensis
catatcaagaatatctgacactctgacacaatcagagagagagagagagagagagagaga

SEQ ID NO: 97 - DNA, 60 bp, Eucalyptus camaldulensis
aatgtattgtatgagccggtatgctaccgacataggggtgcgatcagatgtgatcaaagt

SEQ ID NO: 98 - DNA, 60 bp, Eucalyptus camaldulensis
acgcctaacggtctaagtacagaacacactcgttccctataccgcaaagatactgaaaaa

SEQ ID NO: 99 - DNA, 60 bp, Eucalyptus camaldulensis
tatcgacttacgatggtacttcgagtttgtgaatgcttccttatgaaatggactatcctt

SEQ ID NO: 100 - DNA, 60 bp, Eucalyptus camaldulensis
gaatttctcaaaccacatgagaagttgtaatcttctgaaccatcggctggggttgcagtc

SEQ ID NO: 101 - DNA, 60 bp, Eucalyptus camaldulensis
atcacgtagttttgacatctttgtagacttttaggccttcaaggaaaagtctaaatttgg

SEQ ID NO: 102 - DNA, 60 bp, Eucalyptus camaldulensis
ctatgtactgcgaaaaaagcggtgttgaaataaaactgagataaagctgccaacaggccg

SEQ ID NO: 103 - DNA, 60 bp, Eucalyptus camaldulensis
cccaaaaccacaaggtaattgacgttgccagtgaaggacggcatagttgtatgatctgta

SEQ ID NO: 104 - DNA, 60 bp, Eucalyptus camaldulensis
tttgggaagtgcccttttgtttgcagatcagggaaataaaaatgaactcatgatctcagt

SEQ ID NO: 105 - DNA, 60 bp, Eucalyptus camaldulensis
agagggattgtccttcttgcttgtacatttccgccgacatgcgatatacgtaaagcagaa

SEQ ID NO: 106 - DNA, 60 bp, Eucalyptus camaldulensis
ccataatgtaccaagatccaacgcaacttatattgttaactctcctaccagagtattttt

SEQ ID NO: 107 - DNA, 60 bp, Eucalyptus camaldulensis
ccatgatctttggacgatgaataagaggggttgctaattgcaacccaagcattattctca

SEQ ID NO: 108 - DNA, 60 bp, Eucalyptus camaldulensis
ccttcaaatatgaggacatactatgaacattcggtttgtttgcatctatgcagtgcttgg

SEQ ID NO: 109 - DNA, 60 bp, Eucalyptus camaldulensis
gcgacttgtattttgccttgtcgtagtgtgataatgtaactgtaaatgaggatctggaaa

SEQ ID NO: 110 - DNA, 60 bp, Eucalyptus camaldulensis
gtttgcagtacctactcaaaagagtgtactctttaaagtctgaagatgtaagcgtgtaca

SEQ ID NO: 111 - DNA, 60 bp, Eucalyptus camaldulensis
actccccaactgttaatgctacggtaaatgcgaaagtggtctattgaagtcaataatcca

SEQ ID NO: 112 - DNA, 60 bp, Eucalyptus camaldulensis
ggctggatatatggctccgttacagaggatattctaacgggcttcaagatgcattgtcat

SEQ ID NO: 113 - DNA, 60 bp, Eucalyptus camaldulensis
gcctcttggggaaatacatagcatcggaattttcttttcttttctcttcttctcagtttc

SEQ ID NO: 114 - DNA, 60 bp, Eucalyptus camaldulensis
tctcgtccagtgaccacagtgtatagataaaaatttcgcgcattcactagatatattgag

SEQ ID NO: 115 - DNA, 60 bp, Eucalyptus camaldulensis
cttggttgattattttgcctactttggcaacccagaacaatgaagagagcaatttggccg

SEQ ID NO: 116 - DNA, 60 bp, Eucalyptus camaldulensis
aagcttgaaggcatgatgaacgacatggacagagttaagctggaataaccgcactgtctt

SEQ ID NO: 117 - DNA, 60 bp, Eucalyptus camaldulensis
tgtagtattgtaagttggtcacaggcaggatatggttcaagcttgcgaggatgtgtttcg

SEQ ID NO: 118 - DNA, 60 bp, Eucalyptus camaldulensis
ggcatacttgtaagatggtacatattgtcaaatttacatctgcccattggatgatattgt

SEQ ID NO: 119 - DNA, 60 bp, Eucalyptus camaldulensis
agttccacctgctgttccaagaatcatggtatgtttatgttaacatttatacatctttgc

SEQ ID NO: 120 - DNA, 60 bp, Eucalyptus camaldulensis
caccctctcccttgctttggctttctcggcattcaacagtgaagggctaacaaatttttt

SEQ ID NO: 121 - DNA, 60 bp, Eucalyptus camaldulensis
gctgcaattgtttgctgcaatcgtgagagtattcttgtatccttgcaatatgttgggtca

SEQ ID NO: 122 - DNA, 60 bp, Eucalyptus camaldulensis
atttaagggatacaacatcagggtctgagactgatggtttactgattattctaaggcgag

SEQ ID NO: 123 - DNA, 60 bp, Eucalyptus camaldulensis
tactcacttgcagctgcgcatactcgtactgcgtgatatatctatcttcttctgcaaaaa

SEQ ID NO: 124 - DNA, 60 bp, Eucalyptus camaldulensis
ttcaactgaagttgcagaaattgtgtaccctttagaacagctcatctaactaaaagcatg

SEQ ID NO: 125 - DNA, 60 bp, Eucalyptus camaldulensis
atgtctgttggaatttagcaggaacaggacgcaggaaaagtttttcccttttctcgccct

SEQ ID NO: 126 - DNA, 60 bp, Eucalyptus camaldulensis
gtcggttgattcttttgcgcaagatgtgatataaagtcgtgagcaaaattcattcattat

SEQ ID NO: 127 - DNA, 60 bp, Eucalyptus camaldulensis
ggagctgtaattctaactcttatgtgcttcaaaagattgggacttgggttgaatgcatca

SEQ ID NO: 128 - DNA, 60 bp, Eucalyptus camaldulensis
tccagaagaatgttctggtttacttgttatggatgcacatccagcagattgcttcttacc

SEQ ID NO: 129 - DNA, 60 bp, Eucalyptus camaldulensis
agacgcctgcaactcatattaacatgatgtacatgtttttggtacagcaataaacaagtt

SEQ ID NO: 130 - DNA, 60 bp, Eucalyptus camaldulensis
acaagctctgggtttcattatcgcggtggtttggtaaattttctgattaattcaatggtc

SEQ ID NO: 131 - DNA, 60 bp, Eucalyptus camaldulensis
tgttgcatggataatggggcgccctatggacttgaattttcaactctttgaaacggcagt

SEQ ID NO: 132 - DNA, 60 bp, Eucalyptus camaldulensis
tagtaagcgactatctgaaactgaaatacgctataagaccttcatatctctatgtacact

SEQ ID NO: 133 - DNA, 60 bp, Eucalyptus camaldulensis
gtgccatctcaagagagtgaggaggtgttgtgtgtagacttgaaacaataatggcattga

SEQ ID NO: 134 - DNA, 60 bp, Eucalyptus camaldulensis
acagttactatcaattgcagatgcagaattcacagagctgtgatttcgatgggcttggga

SEQ ID NO: 135 - DNA, 60 bp, Eucalyptus camaldulensis
tcagcagttcagagagagacagaatttctctggctgttttctacacaccagaacccaaca

SEQ ID NO: 136 - DNA, 60 bp, Eucalyptus camaldulensis
ttgttcctggtcacgtctttgaatacattgagtagctatggtgatctgataaatactacg

SEQ ID NO: 137 - DNA, 60 bp, Eucalyptus camaldulensis
ggatggaatttggggaaccatgaagtcctgaatcaatttggaaaactatggctgattgct

SEQ ID NO: 138 - DNA, 60 bp, Eucalyptus camaldulensis
tctggtgtgcaggatatctatatatgatttgcggacatcttgcgagtccatgacaggaac

SEQ ID NO: 139 - DNA, 60 bp, Eucalyptus camaldulensis
atgccatgctatgccatgccatgccagtgtgtgtgaacaggtatggattagagaatttct

SEQ ID NO: 140 - DNA, 60 bp, Eucalyptus camaldulensis
tctaagtgtttgttgctgatttccaaaatatgctgcaccgcggtactgttttttcttgca

SEQ ID NO: 141 - DNA, 60 bp, Eucalyptus camaldulensis
tgagaggaagaatcaagagacaaaatttgctagcttatggttctggcgggtgtgacatga

SEQ ID NO: 142 - DNA, 60 bp, Eucalyptus camaldulensis
atgtaattagaacaaaacgcaatagatggtaaaagctttaaggtgtgaatacgactgtcg

SEQ ID NO: 143 - DNA, 60 bp, Eucalyptus camaldulensis
gttgaagaactactgatcaggagtaattttgatgattatttgattggtgtttgtcccaag

SEQ ID NO: 144 - DNA, 60 bp, Eucalyptus camaldulensis
tgcgtcacggaaatttacagttactctcactgtcaggtctgtgaccggaacatacagttt

SEQ ID NO: 145 - DNA, 60 bp, Eucalyptus camaldulensis
aaagaaagatgaggacgggtttctctacgtcacctacagcggagaaaacacattcggtaa

SEQ ID NO: 146 - DNA, 60 bp, Eucalyptus camaldulensis
tcatgtgcgtttccactgttctacataggtaaaaacatgttaaagtgtcagagagaactc

SEQ ID NO: 147 - DNA, 60 bp, Eucalyptus camaldulensis
ggtttgttccgatatcctttttgagtatgtaacatttttaactgtttatgcattcgaggg

SEQ ID NO: 148 - DNA, 60 bp, Eucalyptus camaldulensis
tggggcattcaccaggtgaagtacctttctaggtaggtcttctcctcttttttccttttt

SEQ ID NO: 149 - DNA, 60 bp, Eucalyptus camaldulensis
ccagctggatcgtttactcaaacattatagttgtatcgtttacacaataccattacaaag

SEQ ID NO: 150 - DNA, 60 bp, Eucalyptus camaldulensis
ctgcgagtatgatttgcattggtttagtagttgtaccacctgattgaactgaagctgcag

SEQ ID NO: 151 - DNA, 60 bp, Eucalyptus camaldulensis
tggaaactggaaacatgatgaactgtagtcctcctttgatcaaagacgttgtaacttgtg

SEQ ID NO: 152 - DNA, 60 bp, Eucalyptus camaldulensis
ccggggttatctcgatgactgttgagagttgtaatatgagaaccaaatgtttggtttgga

SEQ ID NO: 153 - DNA, 60 bp, Eucalyptus camaldulensis
attggatcgacaccgatttgccttcgactaaattcgacggcaacattaagcatttgagcg

SEQ ID NO: 154 - DNA, 60 bp, Eucalyptus camaldulensis
accaccattctacccgcattgcttttgttgagtttatgatggcagaaagtgcgattgctg

SEQ ID NO: 155 - DNA, 60 bp, Eucalyptus camaldulensis
aatccgtaatggctcatgagattttggtaatttgtgtgttcattttgcgaagtgtggcca

SEQ ID NO: 156 - DNA, 60 bp, Eucalyptus camaldulensis
tttgtcagtgtccttgaagagtcttcatttactatggttgtgaattcgaagtgagaaaaa

SEQ ID NO: 157 - DNA, 60 bp, Eucalyptus camaldulensis
ctgctattatgatgcgtgatagaggtgcatatgaacaaagagtgaaagagtactgtgaga

SEQ ID NO: 158 - DNA, 60 bp, Eucalyptus camaldulensis
actgtgaaggtgatctgatatattagttacattccacaggttttgcttctcatagtgaac

SEQ ID NO: 159 - DNA, 60 bp, Eucalyptus camaldulensis
atcaggaccgatgggttcctgatcgtgtatggcttgtgaataaatcctaatttcgtgcac

SEQ ID NO: 160 - DNA, 60 bp, Eucalyptus camaldulensis
atatggtgtgggtgtactagtttgaactttttgcaatatattctgctcaagattaagcac

SEQ ID NO: 161 - DNA, 60 bp, Eucalyptus camaldulensis
gtctgcttgtgtgaattcatgtttggaaacgttgaacggaacaagttcgatagaatatga

SEQ ID NO: 162 - DNA, 60 bp, Eucalyptus camaldulensis
cccaattctcttgtcgaattttgtaaatattttttagtctcttgcccatgggcttcaata

SEQ ID NO: 163 - DNA, 60 bp, Eucalyptus camaldulensis
atttgctctcttgtaagctgtgatttacttgtatgctttgccctgtagttgaattctgta

SEQ ID NO: 164 - DNA, 60 bp, Eucalyptus camaldulensis
gagggccgtcttcccgatgctaccaagggctgtgaccacttgagacaagtgtttggtgtt

SEQ ID NO: 165 - DNA, 60 bp, Eucalyptus camaldulensis
ccctgaacctgatgaccctcaagatgcggtggtaacacaacaggatattacagtatctta

SEQ ID NO: 166 - DNA, 60 bp, Eucalyptus camaldulensis
ttcctcgtgtatcattagatggtaccaaactcatataagctgaaatcagtctataatagc

SEQ ID NO: 167 - DNA, 60 bp, Eucalyptus camaldulensis
tggtgtttgtagctctgacaattcgactgtgttaataaatcctgtgtacagatttgccag

SEQ ID NO: 168 - DNA, 60 bp, Eucalyptus camaldulensis
gctgaaaggctgacttctttttcttttatattttctggggcgtctgtggacgagatgaaa

SEQ ID NO: 169 - DNA, 60 bp, Eucalyptus camaldulensis
ttgtatcggtgatcatctattatcagtgtcaatgactggtaatctttacggaatgtcctt

SEQ ID NO: 170 - DNA, 60 bp, Eucalyptus camaldulensis
cgaaatggtcgcgtaaagcttgcacctttctgaactcttacaagaacgccaactgcaaaa

SEQ ID NO: 171 - DNA, 60 bp, Eucalyptus camaldulensis
tgtgtgtgtgtgtgtgtgtgtgtgtgtgtgtgtgtgtgtgtgtgtgtgtgtgtgtgtgtg

SEQ ID NO: 172 - DNA, 60 bp, Eucalyptus camaldulensis
gaagttcatgagttcgttctcaaagaaaataggcaagttgagcctctttggacacagttc

SEQ ID NO: 173 - DNA, 60 bp, Eucalyptus camaldulensis
cggggtgttcgaaagccacaaaccatctgttgatatatataatttttgactttgcaaaaa

SEQ ID NO: 174 - DNA, 60 bp, Eucalyptus camaldulensis
attagtgtgctgctaagcagaatcatgtgaaatgtaaatgcgatttccatgtcctcaact

SEQ ID NO: 175 - DNA, 60 bp, Eucalyptus camaldulensis
gacgaacttcctgtttgatatgtatgatagttttgctcgtgtaacttcttacctcttttt

SEQ ID NO: 176 - DNA, 60 bp, Eucalyptus camaldulensis
tggatgacaaaatgggagaggccaagatcgacatcaagccgtacttagatgctatagaga

SEQ ID NO: 177 - DNA, 60 bp, Eucalyptus camaldulensis
attgttgtcgtcgtcgtcgtggcgatggtgagaaaatgaaacggggagacaaatgtatat

SEQ ID NO: 178 - DNA, 60 bp, Eucalyptus camaldulensis
taaacagggcgagtttgaagtttgttctccttgaaatagcctttaattggttgttatgag

SEQ ID NO: 179 - DNA, 60 bp, Eucalyptus camaldulensis
aagaggagaagaggctattgagagatagtggtgtttattctggcttcgattcatgaacct

SEQ ID NO: 180 - DNA, 60 bp, Eucalyptus camaldulensis
ggattatgtacagttgtgcattaatttgcatctgtctgttttgcaaatgttatgtacagg

SEQ ID NO: 181 - DNA, 60 bp, Eucalyptus camaldulensis
cgagtctctggaagcaggtgagagaagttggatattgcgctccttattgaatggttcatt

SEQ ID NO: 182 - DNA, 60 bp, Eucalyptus camaldulensis
tctttaaggttcgaggaaaaggtttgcactatggccgaaggaccagcatgtcatatgtga

SEQ ID NO: 183 - DNA, 60 bp, Eucalyptus camaldulensis
tctctctctctctagaggggaagggggcttctaatgtttttgcaagaacacacccaatta

SEQ ID NO: 184 - DNA, 60 bp, Eucalyptus camaldulensis
gtcgtcatggtgtaataataaaatcgtgtgccttttcaaatcttataaacgtcgtgctga

SEQ ID NO: 185 - DNA, 60 bp, Eucalyptus camaldulensis
ggtacactttgtactcagattctcgtagttaatgctaatacgtggtttcagttctataaa

SEQ ID NO: 186 - DNA, 60 bp, Eucalyptus camaldulensis
tgggtcgggttttaactttctcagcagtcgtaaaggctggctagtcaatgcatacgtaaa

SEQ ID NO: 187 - DNA, 60 bp, Eucalyptus camaldulensis
gttttttggcaagctatacatgaatacatggaataaagaaagagaattctgagcaactgg

SEQ ID NO: 188 - DNA, 60 bp, Eucalyptus camaldulensis
gttgggaagggtttttactttagacagacgggatcgttaacaaaaataaagatgtttatg

SEQ ID NO: 189 - DNA, 60 bp, Eucalyptus camaldulensis
ctttgggtgtacatgacagttctgtcaaacatctaagaaacgtgtataagtacagttatg

SEQ ID NO: 190 - DNA, 60 bp, Eucalyptus camaldulensis
aaagcttgtatcctaaatgataatcttaagagttgggtcctagaggaacagatacccatc

SEQ ID NO: 191 - DNA, 60 bp, Eucalyptus camaldulensis
agccctcgcaaatattgaggcggggtgatccttctaatgtggaaaggtgtgtttgttaaa

SEQ ID NO: 192 - DNA, 60 bp, Eucalyptus camaldulensis
ggaaatcttcactaacatgatttcgggcagtataaattacttgcaaagagatggtttcca

SEQ ID NO: 193 - DNA, 60 bp, Eucalyptus camaldulensis
gtcttgttctgtctttgtttatgttggattgaaaatttgaaataaaggacaacgtgaggg

SEQ ID NO: 194 - DNA, 60 bp, Eucalyptus camaldulensis
acgtagaagatgatgtagataaaagatttgtctgtaatggtgaagccatgctaatgtcta

SEQ ID NO: 195 - DNA, 60 bp, Eucalyptus camaldulensis
gatgttcctatctacgcttgagattatgcaagaaaggagatccttcaggaatgctgttct

SEQ ID NO: 196 - DNA, 60 bp, Eucalyptus camaldulensis
ttttttatcatgttcggaatgccgtgatggtcccagaggctatgtacatgtggatttggt

SEQ ID NO: 197 - DNA, 60 bp, Eucalyptus camaldulensis
tggattgttcctgacttatggtccacatgtgaccccatgtgacccatgtccatcaaaaaa

SEQ ID NO: 198 - DNA, 60 bp, Eucalyptus camaldulensis
gagaatctacatgcggttgagttgtctggtccaaggtgacccatgcatagttcaagtaaa

SEQ ID NO: 199 - DNA, 60 bp, Eucalyptus camaldulensis
gaggaataaggcaagaaatacagttcgatggcttttgatgctgtgtgaaagctatttttt

SEQ ID NO: 200 - DNA, 60 bp, Eucalyptus camaldulensis
tttgctgatatgttggagatgcggttttccttgtacagagaacaccaacctatcaaacac

SEQ ID NO: 201 - DNA, 60 bp, Eucalyptus camaldulensis
atcggctgttcgtgaaataaaaaacgaggaaatgaggaccgtctcgtgattaaggaatga

SEQ ID NO: 202 - DNA, 60 bp, Eucalyptus camaldulensis
cagctgagttggtgtagatttttcataagttaccacttgggggaagaggaaggtgatgca

SEQ ID NO: 203 - DNA, 60 bp, Eucalyptus camaldulensis
ctgatgctgccttcagaaccatgtgggctgagtttgagtgggaaaataaggttgctatta

SEQ ID NO: 204 - DNA, 60 bp, Eucalyptus camaldulensis
tgaaatgtgagaatatcatggcatggggcatttgatttgtcatacagtgaagaagttttg

SEQ ID NO: 205 - DNA, 60 bp, Eucalyptus camaldulensis
tgtttgtgcaatcatctttttctggcacatgcaatacaggcggaagttcctttcaaaaaa

SEQ ID NO: 206 - DNA, 60 bp, Eucalyptus camaldulensis
gtttcggggctttgttttaatttccagaactggaagccggactttcgtactccattgtaa

SEQ ID NO: 207 - DNA, 60 bp, Eucalyptus camaldulensis
aaaggatataatgtcacataaacgtacagcctcgctgctgagtgagttgtggataaattt

SEQ ID NO: 208 - DNA, 60 bp, Eucalyptus camaldulensis
ggggctttctcccttttccttttgattagaactggtcaagagcaggaatggaatgcagta

SEQ ID NO: 209 - DNA, 60 bp, Eucalyptus camaldulensis
ggattactcagccttgttgtggggggactaggttaagtggaagttcgattcagtaaaaaa

SEQ ID NO: 210 - DNA, 60 bp, Eucalyptus camaldulensis
atcttatcggaacatggtgctgttgttgatagttgatagcatgggaagcattttcttgag

SEQ ID NO: 211 - DNA, 60 bp, Eucalyptus camaldulensis
atttcaacttcatatcaagccttattgtttggaagacatctctagatatctcaggttagg

SEQ ID NO: 212 - DNA, 60 bp, Eucalyptus camaldulensis
ctcaagcactcgcaagtgatattaatgctaacataggccggttgtcttctgtatagtgaa

SEQ ID NO: 213 - DNA, 60 bp, Eucalyptus camaldulensis
attcgctgagcatgtcatcttgcctctcgagtttcggatttatcaaattagcttttgact

SEQ ID NO: 214 - DNA, 60 bp, Eucalyptus camaldulensis
gcttcaggatggtgagaccacaaatgtatttattatcaaaaagaatgtttttgagttgcc

SEQ ID NO: 215 - DNA, 60 bp, Eucalyptus camaldulensis
ttgcttttgttatggtctcaaaagtggatgtataggttgttaggaatatttcttctggag

SEQ ID NO: 216 - DNA, 60 bp, Eucalyptus camaldulensis
tcggtttatggaagggggaaaggaatatggatgcacatcgggcggttttcttatgattct

SEQ ID NO: 217 - DNA, 60 bp, Eucalyptus camaldulensis
gtttcataaacgagccctgtaaatatacatgcaagtatatctggagtattggaagcactt

SEQ ID NO: 218 - DNA, 60 bp, Eucalyptus camaldulensis
ctgtagcttcaggacaccaccacggttattttgtccgtcaacttatagaaatacagtata

SEQ ID NO: 219 - DNA, 60 bp, Eucalyptus camaldulensis
accccaaaaccccaattcattgtagaaggatatttgcataagcatttaaggtgaactttt

SEQ ID NO: 220 - DNA, 60 bp, Eucalyptus camaldulensis
actgggttcttcccctaaactctactaaaccttgtgtaattgttgttcttatggtgttaa

SEQ ID NO: 221 - DNA, 60 bp, Eucalyptus camaldulensis
ggcctttgatcatctctctctctttaatctgtggatcttatgtctcttcgtttgagtagt

SEQ ID NO: 222 - DNA, 60 bp, Eucalyptus camaldulensis
aaccttaaattttggaagggtggagctgggacaggcataaagctacccggtatgtgtaat

SEQ ID NO: 223 - DNA, 60 bp, Eucalyptus camaldulensis
acatctgaattgacagtgttcatattttcttctctcgataaattatctcaatgaaaaaaa

SEQ ID NO: 224 - DNA, 60 bp, Eucalyptus camaldulensis
tgggggatgcctgactaatattcttttgtaacagaagtttggattaagaagactctttaa

SEQ ID NO: 225 - DNA, 60 bp, Eucalyptus camaldulensis
gtttgcaacagcggctgtgagaaactgagtaggtatgggttagcttcccattttgtgtat

SEQ ID NO: 226 - DNA, 60 bp, Eucalyptus camaldulensis
tagagaggattattcgtccgtgtttggttgagctgaacgaggacccagatgttgatgtta

SEQ ID NO: 227 - DNA, 60 bp, Eucalyptus camaldulensis
gcaagaccataacccttgaggttgaaagttcggacactatcgataacgtgaaggcgaaaa

SEQ ID NO: 228 - DNA, 60 bp, Eucalyptus camaldulensis
tccgagtttcagtgacaaatccccatgtttggaatttgagtcgtttgtaccagctttgca

SEQ ID NO: 229 - DNA, 60 bp, Eucalyptus camaldulensis
ttatccatgagttggcttttcaaatcaacattagctctcttgtgatattgagcaaaactg

SEQ ID NO: 230 - DNA, 60 bp, Eucalyptus camaldulensis
ttgtttgtgtgtgtaccgagttaatcaagaaaagggacggcaggtctcaaggatgaggaa

SEQ ID NO: 231 - DNA, 60 bp, Eucalyptus camaldulensis
gtgtgaggctgaggtctttgtttttaaaacacctcagacaaggaactatgggagcgacaa

SEQ ID NO: 232 - DNA, 60 bp, Eucalyptus camaldulensis
atgctgatttgaggtgttaaaactccggattttcttgcgaactatgctgagctaagcatt

SEQ ID NO: 233 - DNA, 60 bp, Eucalyptus camaldulensis
atccataccagatcaatccatagaagagatgatagccaatgggaggcaggtaaggtgctt

SEQ ID NO: 234 - DNA, 60 bp, Eucalyptus camaldulensis
gagcataaaatatctggggatggacactcttcttaatttgtttgttgcaacctgtataaa

SEQ ID NO: 235 - DNA, 60 bp, Eucalyptus camaldulensis
tgtgggtgaagaacggattgtgggtttggattgatatacatgaatagccagttgtactca

SEQ ID NO: 236 - DNA, 60 bp, Eucalyptus camaldulensis
tgggagaagaaaattctccagagagaccagttaatcaagacttgttgcagttgtttgtgt

SEQ ID NO: 237 - DNA, 60 bp, Eucalyptus camaldulensis
cccccgtggaagatgttgaagctagtgaaataactgttgctgcaaatgatgtggaagaca

SEQ ID NO: 238 - DNA, 60 bp, Eucalyptus camaldulensis
aattataaaatttctgatgtttccaaagatcatcagctgacgactgtctcagtcggtgag

SEQ ID NO: 239 - DNA, 60 bp, Eucalyptus camaldulensis
tgggctattgggcatgtataaggtttgatgttatacgtcctttcaaggagctgttgatat

SEQ ID NO: 240 - DNA, 60 bp, Eucalyptus camaldulensis
aactgtgatcttggttgtgcagaattcaatgtactgaataaataagatgctaattgccga

SEQ ID NO: 241 - DNA, 60 bp, Eucalyptus camaldulensis
gcttggatttccatattttctttctccttattcctgtaaatattctcgacgggaaaagcg

SEQ ID NO: 242 - DNA, 60 bp, Eucalyptus camaldulensis
gggctggttgttccatacatggtagttttccggtggcaacagaaaattcggtttttgcta

SEQ ID NO: 243 - DNA, 60 bp, Eucalyptus camaldulensis
tttccctggacattaatgtcaatattgtatgaggattatattcggtttgaggtttgttcc

SEQ ID NO: 244 - DNA, 60 bp, Eucalyptus camaldulensis
gtttgggaaggacttttgcaaagtcccttgtacctccaaagtccattgggcaaatgtgtt

SEQ ID NO: 245 - DNA, 60 bp, Eucalyptus camaldulensis
cccaaaaattcctggctttgtgtacttgtaaaatggtacgagaaagaaagagttttccct

SEQ ID NO: 246 - DNA, 60 bp, Eucalyptus camaldulensis
tgaggcaatgtcacatcggatgcatatagatcacagtgtcaaactaattggaaagctctt

SEQ ID NO: 247 - DNA, 60 bp, Eucalyptus camaldulensis
actatagctaggatcactgcttgtgtacttgagtagattagcgaacaatgttgatgcttt

SEQ ID NO: 248 - DNA, 60 bp, Eucalyptus camaldulensis
ttgtgtgcttttgcgttccagtatatccaaaaatatgtaatcagctctcactttttcagt

SEQ ID NO: 249 - DNA, 60 bp, Eucalyptus camaldulensis
cggtggttgttgactttcttgaagctggtcacgaacgggtggccaagggggaagactaga

SEQ ID NO: 250 - DNA, 60 bp, Eucalyptus camaldulensis
gtgcaccatgtgcttctgtcaattaatatcttttctaggatgatatggttaatgcatcct

SEQ ID NO: 251 - DNA, 60 bp, Eucalyptus camaldulensis
tggaggatctgtattttggcttcccttttgactgctgccagaacagttaatgtatggtgc

SEQ ID NO: 252 - DNA, 60 bp, Eucalyptus camaldulensis
aagatgattatgagttggtcgatcagattctgaaagcaacgtgtcagtcgtccggcaaga

SEQ ID NO: 253 - DNA, 60 bp, Eucalyptus camaldulensis
acacttatgcattgaagtatagcagtaggcaagtgtagccactcttgcttgtcctgtgaa

SEQ ID NO: 254 - DNA, 60 bp, Eucalyptus camaldulensis
ggtttgactacggcgtggtggctctcggggtatatcagaactgaatttctaaagtataca

SEQ ID NO: 255 - DNA, 60 bp, Eucalyptus camaldulensis
ttttttggcttgaactgtttttctggcataaaagcgataaagaatctcgtcttggtttac

SEQ ID NO: 256 - DNA, 60 bp, Eucalyptus camaldulensis
tgcacagaaagtgcttctaacttttcaaggagaaggtcattgagggaagagattactgtc

SEQ ID NO: 257 - DNA, 60 bp, Eucalyptus camaldulensis
gtaggccttgagggcaataaaagtaaatttagctgtgatttggaacatccttttacctgc

SEQ ID NO: 258 - DNA, 60 bp, Eucalyptus camaldulensis
aaggcgtgagagagaagcacctaattccgtttcttttctttcggttcatttgggtccttc

SEQ ID NO: 259 - DNA, 60 bp, Eucalyptus camaldulensis
ttaatggacggagtttgctgtgtaattagagtgaatatatatccaagttggctacttctc

SEQ ID NO: 260 - DNA, 60 bp, Eucalyptus camaldulensis
taggagaagtggttttctttttatacttaaatagaactttcccgagcgtgtttttgtcca

SEQ ID NO: 261 - DNA, 60 bp, Eucalyptus camaldulensis
tagaacccgcaaacatgcttgggtttacttcatagatgaatttatgttctttttgggccg

SEQ ID NO: 262 - DNA, 60 bp, Eucalyptus camaldulensis
cagcatgtaatataatttcgattcgaatcaaatccccggtcacataatctctgatcttct

SEQ ID NO: 263 - DNA, 60 bp, Eucalyptus camaldulensis
ggtataaagaagtgagactcgtgattaaggagtcgaagcatcgtggaggagatcctctaa

SEQ ID NO: 264 - DNA, 60 bp, Eucalyptus camaldulensis
cttgttacgattaaatttgagatgaccgacttgctaattagaagcatcgctatgatggat

SEQ ID NO: 265 - DNA, 60 bp, Eucalyptus camaldulensis
ggcttggataccaggaaaacataagttttgcagaccctaaggatgcaggagtcatccatt

SEQ ID NO: 266 - DNA, 60 bp, Eucalyptus camaldulensis
tgggattgccgatgtcatgacctgatggtttatcgtcatgaattgctatcaaatcattat

SEQ ID NO: 267 - DNA, 60 bp, Eucalyptus camaldulensis
tttgtgcagtgtagtaacaatattgcaggatgtgttttattagtatttggaaatggcctc

SEQ ID NO: 268 - DNA, 60 bp, Eucalyptus camaldulensis
ggatcagttaactgggttgttccgtgattggtttctgtttgttttttgcattgtctcatt

SEQ ID NO: 269 - DNA, 60 bp, Eucalyptus camaldulensis
cctgaccttttgactatcagttctgcgcttgaggtcaaataagatttatactgcatggca

SEQ ID NO: 270 - DNA, 60 bp, Eucalyptus camaldulensis
acacttatgcattgaagtatagcagcaggcaagtgtagccactcttgcttgtcctgtgaa

SEQ ID NO: 271 - DNA, 60 bp, Eucalyptus camaldulensis
gcttgcttttctaacagttagtggcagaatctgagtttacacaattacattagggagatg

SEQ ID NO: 272 - DNA, 60 bp, Eucalyptus camaldulensis
aggagagcgttgtattcaaatatctgatggtaggatatgacatcttaggtccaacttcat

SEQ ID NO: 273 - DNA, 60 bp, Eucalyptus camaldulensis
cgagcctcagtctattatagtttccattaaactgcagcttggagatgagatttacttgcg

SEQ ID NO: 274 - DNA, 60 bp, Eucalyptus camaldulensis
gatctttgctgcagtttgtgactttgaacagaataatggcttgtgccaatgtttgaccgg

SEQ ID NO: 275 - DNA, 60 bp, Eucalyptus camaldulensis
tttataagtttttgttttttaagaatgaagggacggacggaccggaccattttggaaaaa

SEQ ID NO: 276 - DNA, 60 bp, Eucalyptus camaldulensis
tttttttctttcatttttcctaaatcaatcgatcgcattgcgagaaatatagtattgtct

SEQ ID NO: 277 - DNA, 60 bp, Eucalyptus camaldulensis
gggtcaaagaagaagaaacagaagaagaaagaccccaatgcaccaaaaaaggctatgtct

SEQ ID NO: 278 - DNA, 60 bp, Eucalyptus camaldulensis
atgtgttggaggaattttcagatggctttgtacaatgggtaacctatggagaggcgctct

SEQ ID NO: 279 - DNA, 60 bp, Eucalyptus camaldulensis
atggctgggagatgcttggaatatgtatgtaccatcattagttccatcaaagagacatca

SEQ ID NO: 280 - DNA, 60 bp, Eucalyptus camaldulensis
gaggatgcgaacgttagtcatcctaaacctatttatgtattttctgtgatgggagccaaa

SEQ ID NO: 281 - DNA, 60 bp, Eucalyptus camaldulensis
tgttctgatgcgattgtatgatgacgcgatgagcagaataaaagtcgcactttccttgtg

SEQ ID NO: 282 - DNA, 60 bp, Eucalyptus camaldulensis
tagcagctagaaaacatagatgaaaggaggaatttctcctctagccttctctttaaaaaa

SEQ ID NO: 283 - DNA, 60 bp, Eucalyptus camaldulensis
ttggggcaacttctgtgtataccggcttcatgaacagatgtgcatttccacagcgtaaat

SEQ ID NO: 284 - DNA, 60 bp, Eucalyptus camaldulensis
atccgagagcacttaagctgtcaaattgtgagagagcaatgcgttgacaatggatgtatc

SEQ ID NO: 285 - DNA, 60 bp, Eucalyptus camaldulensis
ttgagattcttagtgtggttgtaagggtaattcgattggcaatgtgatggctacattcaa

SEQ ID NO: 286 - DNA, 60 bp, Eucalyptus camaldulensis
accatccatctccttttgataagcgttgactttgttttacgatgaatcggtgcttgtacg

SEQ ID NO: 287 - DNA, 60 bp, Eucalyptus camaldulensis
gaccaatgtacaggttacggctctgctgtccctttgtgtctaataaaagctttggttcaa

SEQ ID NO: 288 - DNA, 60 bp, Eucalyptus camaldulensis
cttatttgattttatcttttgaccaatgtacaggttacggctctgctgtccctttgtgtc

SEQ ID NO: 289 - DNA, 60 bp, Eucalyptus camaldulensis
tgatggttgtggtgggactgagtgatttcagagtaacatgatgtatgagcatcagcctac

SEQ ID NO: 290 - DNA, 60 bp, Eucalyptus camaldulensis
gaaggtgaagcccgaagggaaggtcactattatgcccaagcattattggtgatcctattt

SEQ ID NO: 291 - DNA, 60 bp, Eucalyptus camaldulensis
gccattcattgtagaatgacagtatcaacattagtctctgctattgactattgcaaggaa

SEQ ID NO: 292 - DNA, 60 bp, Eucalyptus camaldulensis
gctgattgctagtttgcgcacggtttgatttctgtgtacataacctagttctatggaaaa

SEQ ID NO: 293 - DNA, 60 bp, Eucalyptus camaldulensis
gccccgacattcaaggcatcattttatctgcctttgtcattggattgtaacagcatcgag

SEQ ID NO: 294 - DNA, 60 bp, Eucalyptus camaldulensis
tcagagttcgtcattttatggacagttgcaattgttttgcgtcaagagaaatggtttggt

SEQ ID NO: 295 - DNA, 60 bp, Eucalyptus camaldulensis
tggctctaaaatggggtactctttctttatttatggaaaatgacgaatatcacgaattcc

SEQ ID NO: 296 - DNA, 60 bp, Eucalyptus camaldulensis
ttggtagagacctgtttctgtctgttataatcttcactcttgttccgagcatgctgtgcc

SEQ ID NO: 297 - DNA, 60 bp, Eucalyptus camaldulensis
gaagaaagaagaagtatcagcagaggctgttgatgcttcaatgccagaagctgaagcaga

SEQ ID NO: 298 - DNA, 60 bp, Eucalyptus camaldulensis
gacgcgttttactgctgagcatgtttaagcgtactttcacctcttggcaggataaataac

SEQ ID NO: 299 - DNA, 60 bp, Eucalyptus camaldulensis
gcttttcgtgtcagcgtgaagaagccgaggcagactctttggtaatattcagttgtgaaa

SEQ ID NO: 300 - DNA, 60 bp, Eucalyptus camaldulensis
caactgtggcacaccatatttgctgtggaaattatgtatgtgggtgatgtgttaagaagg

SEQ ID NO: 301 - DNA, 60 bp, Eucalyptus camaldulensis
gatggccactgaaatagacagtttgacatgatatttccgtctcatgggcacgcacacaaa

SEQ ID NO: 302 - DNA, 60 bp, Eucalyptus camaldulensis
attgatgtagcatatttttcttctatatcctttacggttctcgcttcgccggcttacgat

SEQ ID NO: 303 - DNA, 60 bp, Eucalyptus camaldulensis
ttcctagagtgttggtaccacatctgggttcgcgtacatttcttctgttgaatccctttg

SEQ ID NO: 304 - DNA, 60 bp, Eucalyptus camaldulensis
gcaagctacggtttcttgcgaccattttttctctatggaagctctggttgttgctcaaga

SEQ ID NO: 305 - DNA, 60 bp, Eucalyptus camaldulensis
tcattctgttcacatcgatgaaaggtggctcaaatgtcgaatgtcggaagatcttccttg

SEQ ID NO: 306 - DNA, 60 bp, Eucalyptus camaldulensis
atggaacaggattatgacatgggctccaccatccgcgataagataattcctcatgctgta

SEQ ID NO: 307 - DNA, 60 bp, Eucalyptus camaldulensis
ttaaactcgttagacatatttgttcagctaaattagtgagattgagattgagattggtat

SEQ ID NO: 308 - DNA, 60 bp, Eucalyptus camaldulensis
ctggttttcctgtttcggtggtgattattgtcaacgtgtgagagttgagatagaggagca

SEQ ID NO: 309 - DNA, 60 bp, Eucalyptus camaldulensis
agtgaaagccaagcttggcaaggttgacgagtacttcaacaagctggcagatgccatggt

SEQ ID NO: 310 - DNA, 60 bp, Eucalyptus camaldulensis
atgcttgtcattaacaggacaatgacaaaacatcagtgtgaatcacgtcaacttcgcaga

SEQ ID NO: 311 - DNA, 60 bp, Eucalyptus camaldulensis
tagctttagattttccaagctagcaaaggctacttgctgtatttgaccagcgaatttgat

SEQ ID NO: 312 - DNA, 60 bp, Eucalyptus camaldulensis
gcttcccgagcgaagttaaattcagtccagccagccttaacctcaatatgagaactgttt

SEQ ID NO: 313 - DNA, 60 bp, Eucalyptus camaldulensis
gggtgagatacgtcactcaagctaattcctatgtggctcgaatgtgaggttattgagctt

SEQ ID NO: 314 - DNA, 60 bp, Eucalyptus camaldulensis
gctgcctgtatgcattcatatttttctttggttccatactgaaaagacatgtttgtgaag

SEQ ID NO: 315 - DNA, 60 bp, Eucalyptus camaldulensis
cgtgatgatgctcgtggggtttgcattgggataacaaggtttacaaacaaggctcacatt

SEQ ID NO: 316 - DNA, 60 bp, Eucalyptus camaldulensis
ttgttttctcaccgattgctcactttgtagttcttataaactcggtattcagcaataatc

SEQ ID NO: 317 - DNA, 60 bp, Eucalyptus camaldulensis
caagttcactgcaatgctcatcatccatcgacgagatttgatcaggtttggagctcatgt

SEQ ID NO: 318 - DNA, 60 bp, Eucalyptus camaldulensis
tgtagttccatgtgtcattcttttgtttctcagcctataagtgttatctgttgttgctaa

SEQ ID NO: 319 - DNA, 60 bp, Eucalyptus camaldulensis
tgcttatttcctgtgtgctttgtttaattaatacacccttttgtgagctgttgctcactc

SEQ ID NO: 320 - DNA, 60 bp, Eucalyptus camaldulensis
acttggaacggtacatggccgtggatgttgacagagttttgcactgagaagttttctgtt

SEQ ID NO: 321 - DNA, 60 bp, Eucalyptus camaldulensis
ttttgttcagccggctttttatgaagcttttggacttacagttgttgaggctatgacgtg

SEQ ID NO: 322 - DNA, 60 bp, Eucalyptus camaldulensis
cctatcgaatgggatcttcttccagaaataaatgaaatttccattcaaaggagccgaaat

SEQ ID NO: 323 - DNA, 60 bp, Eucalyptus camaldulensis
gcgttaatagtaccatcctcatgcaaggactcccgggattgaactatcttgtagcagaaa

SEQ ID NO: 324 - DNA, 60 bp, Eucalyptus camaldulensis
tctccctctctccctctcttctctcaactatgcagggttttggttgagatgattgtcatt

SEQ ID NO: 325 - DNA, 60 bp, Eucalyptus camaldulensis
agaggagctgtcttactgtaccgcatcatctgcggagatttcactgagaatagatttgct

SEQ ID NO: 326 - DNA, 60 bp, Eucalyptus camaldulensis
tgtgttgctcttggagcttttaactgggaaagccccggccaattctgtcttaaatgaaga

SEQ ID NO: 327 - DNA, 60 bp, Eucalyptus camaldulensis
aatagtatgtggatgccatggtggaccaaagcaacaaggtgctgttcaaatcaccatgca

SEQ ID NO: 328 - DNA, 60 bp, Eucalyptus camaldulensis
cagagcgagtgcctgaggctctcgaattcctctgatgcatcttagtagttttatactgta

SEQ ID NO: 329 - DNA, 60 bp, Eucalyptus camaldulensis
tggatggctgtagtgaagaggaaagctcattctgcttgcaggagtctacaaacgttttag

SEQ ID NO: 330 - DNA, 60 bp, Eucalyptus camaldulensis
cagagtatttttagactctgaagttatgtgaaagtaaagaaactgatttccctgaaaaaa

SEQ ID NO: 331 - DNA, 60 bp, Eucalyptus camaldulensis
gcaccctctcccttgctttggctttttggctttctaagcattcagcagtgaaaagctatt

SEQ ID NO: 332 - DNA, 60 bp, Eucalyptus camaldulensis
gtgcggaaccggcatgcgtaacataccaaattttaattgttacctttttaagaatcaaag

SEQ ID NO: 333 - DNA, 60 bp, Eucalyptus camaldulensis
ctcgaattctgtatgcggtacaatcactagaaatttgaattaatgctaaatccatggatg

SEQ ID NO: 334 - DNA, 60 bp, Eucalyptus camaldulensis
acggtgttgctatcgatccggtaattctatagtaggcctagaggattgaagggatgaaga

SEQ ID NO: 335 - DNA, 60 bp, Eucalyptus camaldulensis
tggcttttggacctcgatgagtttggaaatggctggcactaagctctattatgatgctat

SEQ ID NO: 336 - DNA, 60 bp, Eucalyptus camaldulensis
tcaaacccgttaaaacgttgtactcttaagagtagtgtattcagccctttgtatttgatt

SEQ ID NO: 337 - DNA, 60 bp, Eucalyptus camaldulensis
gtcattcgtatggcggttgtaaattaaacaaatagcgaactacctggtagtgtaaattat

SEQ ID NO: 338 - DNA, 60 bp, Eucalyptus camaldulensis
ttggtacctttgaaatggatgtttgtttttccttacttttcttattgtaagaagcgcgtc

SEQ ID NO: 339 - DNA, 60 bp, Eucalyptus camaldulensis
ggaatctgcccagaaacggatgatctttcttttccatgagatgtgatgtgttcctaattc

SEQ ID NO: 340 - DNA, 60 bp, Eucalyptus camaldulensis
ggggagcatttgaggtttagtgtcatcatttatgtaaagggatggtaatgttttgttggc

SEQ ID NO: 341 - DNA, 60 bp, Eucalyptus camaldulensis
tgtgggccatgctggcgattagtccaatatggtcgctgcgcatataagtttttaaggatt

SEQ ID NO: 342 - DNA, 60 bp, Eucalyptus camaldulensis
tctgcattggaatgttattatgattaatgaaactctggttgtgatccgatcgcttcaaaa

SEQ ID NO: 343 - DNA, 60 bp, Eucalyptus camaldulensis
tacgtcttcaaataagtaggcatatggggctttggcagattgcctatcgggatgtctatt

SEQ ID NO: 344 - DNA, 60 bp, Eucalyptus camaldulensis
gtcgtttgacaaaaatgtaaacggttggaactctttgcggcaaggcattatgatttatca

SEQ ID NO: 345 - DNA, 60 bp, Eucalyptus camaldulensis
aagaggatggaaagtgagtaaaactatcattgccctcttgcgcatcaacctcatgctttt

SEQ ID NO: 346 - DNA, 60 bp, Eucalyptus camaldulensis
caagacttttgagtgcctgatttcatctatatccattgactgatttgagttttcagaacg

SEQ ID NO: 347 - DNA, 60 bp, Eucalyptus camaldulensis
ggaactggagacactttacgttcatttggaagacgaacatgaatccaataggtcttggtt

SEQ ID NO: 348 - DNA, 60 bp, Eucalyptus camaldulensis
agtgaagaaaatatcagacttgagaaggatgtgtgtggagaagggagtcaacccatggat

SEQ ID NO: 349 - DNA, 60 bp, Eucalyptus camaldulensis
aaatctgtgcccattgtgaagggaggtgagcagacaaaaatggaagagggtgaatttttc

SEQ ID NO: 350 - DNA, 60 bp, Eucalyptus camaldulensis
cccaacaagaggaagagaagaaagtctctccggtgggtgattttgatgtgttagcttgaa

SEQ ID NO: 351 - DNA, 60 bp, Eucalyptus camaldulensis
atgaaagacaatacgagtctctctctcctgagaaacaagtcgcacactgaggttggagtt

SEQ ID NO: 352 - DNA, 60 bp, Eucalyptus camaldulensis
aaggtcatcaatgacaggtttggcattgttgagggacttatgaccactgtccactccatt

SEQ ID NO: 353 - DNA, 60 bp, Eucalyptus camaldulensis
tgttagccttcttcagtgggatctattcaacatatctttccaatagtttatctagaatgg

SEQ ID NO: 354 - DNA, 60 bp, Eucalyptus camaldulensis
gtagttggtcgtacgagatgagaacataaggcgtgggagtattgtaaattattccgttgg

SEQ ID NO: 355 - DNA, 60 bp, Eucalyptus camaldulensis
agataaggcagactcttgtatatttatgttaaactggatgatttgtagcttgtgcgttag

SEQ ID NO: 356 - DNA, 60 bp, Eucalyptus camaldulensis
tcgaacgaatccattgacacaaattgttcatgggagaaaattgacgaactattggcaaga

SEQ ID NO: 357 - DNA, 60 bp, Eucalyptus camaldulensis
gtgtctgcgtctataaactattgggagcaccttcatagcaatgacatccgtgattcaact

SEQ ID NO: 358 - DNA, 60 bp, Eucalyptus camaldulensis
gatgatgatttgagtgatgatgaacagttttacaagaatacaagtgtgaacaatggtgcc

SEQ ID NO: 359 - DNA, 60 bp, Eucalyptus camaldulensis
taactgttttttgaagaaggcatggtggtggaactctattgaaggcagaggtttcacagt

SEQ ID NO: 360 - DNA, 60 bp, Eucalyptus camaldulensis
tttcccttctggggatcatcaacgtctacacaggcgtaattgcctttcacaacaagacat

SEQ ID NO: 361 - DNA, 60 bp, Eucalyptus camaldulensis
cgcgatgcgtcatctttgggagatggatatccttggccccttttttcctttaaacctttt

SEQ ID NO: 362 - DNA, 60 bp, Eucalyptus camaldulensis
tcctgcatgagaggctcccttgtttaagctcattttgaatgaaatattccgcgagtcgct

SEQ ID NO: 363 - DNA, 60 bp, Eucalyptus camaldulensis
tgaagattcgtttaattcgttgaaggaaacattggctgctgtataattgggaaccaagaa

SEQ ID NO: 364 - DNA, 60 bp, Eucalyptus camaldulensis
atgatggatgtgtactaaccttaccggggaaagatcactcaataaacacgagagcgagtt

SEQ ID NO: 365 - DNA, 60 bp, Eucalyptus camaldulensis
ggtcatatatgtgtttatgtaattcgatctcctactgcactagacaactatcttgaagca

SEQ ID NO: 366 - DNA, 60 bp, Eucalyptus camaldulensis
tctggttttctttttcaagctttgctttgtatgtattttattagagggtgttgttgtccg

SEQ ID NO: 367 - DNA, 60 bp, Eucalyptus camaldulensis
tggaaatgtctgtttactcctccacgaacaatcactttgtataaaatgcagacccctgct

SEQ ID NO: 368 - DNA, 60 bp, Eucalyptus camaldulensis
atcctgtggggtacaaacccttgacaagagcaggagatacaaagcattaaggacgaagat

SEQ ID NO: 369 - DNA, 60 bp, Eucalyptus camaldulensis
tccggttattgcactttgtggaagtgtatccttgtcatttgaggatcatttgacgagtca

SEQ ID NO: 370 - DNA, 60 bp, Eucalyptus camaldulensis
ccgggtcagctgatcgcaccagaaaggagatctcaaaatcaagcattgatgaattgagaa

SEQ ID NO: 371 - DNA, 60 bp, Eucalyptus camaldulensis
ggttcaatattcatctaaaggttgaaaagaggaaggcttgttagagggctaaaggaaatt

SEQ ID NO: 372 - DNA, 60 bp, Eucalyptus camaldulensis
attctgacagacattgtggtactttgctttcctacttgtgttttcttttctttgaaaagc

SEQ ID NO: 373 - DNA, 60 bp, Eucalyptus camaldulensis
gccgtgttgtcttgggagcacaacattgagctcgagtctgtcattgtcgttttatttctt

SEQ ID NO: 374 - DNA, 60 bp, Eucalyptus camaldulensis
taacttcagtgttattaccctggacgaggaacagattgcagcaaatcttgtcaagaacgg

SEQ ID NO: 375 - DNA, 60 bp, Eucalyptus camaldulensis
tagttcctacttttgtgaatcctgactcattatctgaaggatagaggattagcacaaaaa

SEQ ID NO: 376 - DNA, 60 bp, Eucalyptus camaldulensis
gttgaatgtgtcatccttggtaaatacaactccatgacttccatcgaacgaatgcattaa

SEQ ID NO: 377 - DNA, 60 bp, Eucalyptus camaldulensis
tggccatacgagtaaaatttcagacttttcatggaacacctgtgaagactgggttgtagc

SEQ ID NO: 378 - DNA, 60 bp, Eucalyptus camaldulensis
atgtgctgatgcccatcatgtcttttataactagctttgataccgcttatatctggactg

SEQ ID NO: 379 - DNA, 60 bp, Eucalyptus camaldulensis
aatatgagttgtgacagaattcaaactcagaaattctaataatcgcccacaggttacagc

SEQ ID NO: 380 - DNA, 60 bp, Eucalyptus camaldulensis
tcacgtgttttttcgcctaatttattgtcgctgcagcagtttgcggtcatttgctgctgt

SEQ ID NO: 381 - DNA, 60 bp, Eucalyptus camaldulensis
taaccatggtctatgggcatgtgttttcccagcttgctatatctttgctaaatgataagt

SEQ ID NO: 382 - DNA, 60 bp, Eucalyptus camaldulensis
cagctgacttggccatggagccaatcacaaatctctcaatgaaaagcgcagaaaaaggta

SEQ ID NO: 383 - DNA, 60 bp, Eucalyptus camaldulensis
cagcgagccggcgctgaagcaatttagaaaacattttctttcttcttctttctgtccttt

SEQ ID NO: 384 - DNA, 60 bp, Eucalyptus camaldulensis
ttatcataggagtcattattttgggttgaagatctgggacagagaagcatcagtctgttg

SEQ ID NO: 385 - DNA, 60 bp, Eucalyptus camaldulensis
gtgtctactatccaggcttgcccagtcatcctgaacatcagattgccaagaaacaaatga

SEQ ID NO: 386 - DNA, 60 bp, Eucalyptus camaldulensis
attcatgactaaaactatgagatggctctttaaagctggaaatggagaaacttgcgatac

SEQ ID NO: 387 - DNA, 60 bp, Eucalyptus camaldulensis
gcacgtgcggctagaccatcttcatacaagtttgtaagagcttttcgtctgtacccaaat

SEQ ID NO: 388 - DNA, 60 bp, Eucalyptus camaldulensis
cgaaaaatgaggaccagttaagaggttcatgttgggagtggatcctggtgtgaccatttg

SEQ ID NO: 389 - DNA, 60 bp, Eucalyptus camaldulensis
tgttttcttctggccagactgtaataacatagggcagccacacttaagcggggaaagaat

SEQ ID NO: 390 - DNA, 60 bp, Eucalyptus camaldulensis
agtgactgaaccactatgcctgcaactgctggagtaaaagcaaaggaatcaagctgcaaa

SEQ ID NO: 391 - DNA, 60 bp, Eucalyptus camaldulensis
ttctcttccaggtgttgaacttttgttgctcaatctgaatgaatttcatgattgaagaac

SEQ ID NO: 392 - DNA, 60 bp, Eucalyptus camaldulensis
gttatgcaggcgtttactgtttacatcttgtctaaggtaaaatgcacagcatagacttaa

SEQ ID NO: 393 - DNA, 60 bp, Eucalyptus camaldulensis
atggatttgtctacatatcaagtgagaatagcgagacagtaaaacagagatgagaagacg

SEQ ID NO: 394 - DNA, 60 bp, Eucalyptus camaldulensis
tgtaacattgtacggtctcaatgaagggaagagggacttgtcacggcagaaatggtctta

SEQ ID NO: 395 - DNA, 60 bp, Eucalyptus camaldulensis
ttccctttcatttctttttcctctcaactcttttaccagacgaacttgtgaaaaggaatg

SEQ ID NO: 396 - DNA, 60 bp, Eucalyptus camaldulensis
ttttcggagatagctgtgaaatgtcctgactgggtaactgctaaataaatccaccccctt

SEQ ID NO: 397 - DNA, 60 bp, Eucalyptus camaldulensis
gtgatgggtgctcggttacctaggaaagctgttgtttaacgtagtatgttcgtgtgctta

SEQ ID NO: 398 - DNA, 60 bp, Eucalyptus camaldulensis
tcgagcgtaagaatgtgaaagaaacgccttgctaattcattgcagcagcttcgtccagaa

SEQ ID NO: 399 - DNA, 60 bp, Eucalyptus camaldulensis
atttggtttctggacaattttacttgtacatttggattgatcagttgtaatgccaatgcg

SEQ ID NO: 400 - DNA, 60 bp, Eucalyptus camaldulensis
ttagctagtgttgcacgtatttcccaccaaaagaatatgtaatccgatactgacggagat

SEQ ID NO: 401 - DNA, 60 bp, Eucalyptus camaldulensis
aggagaagtgtcggcattctacggatctcccttttgagaattaaaaattctcaccagcgg

SEQ ID NO: 402 - DNA, 60 bp, Eucalyptus camaldulensis
gtgcttcggggagggtcttcttgtcttaagctgtattaatttcctactgccgaactttct

SEQ ID NO: 403 - DNA, 60 bp, Eucalyptus camaldulensis
gacaaattgaagaggtggtgcagcatcctttccttgataaggatgagcttgatgcaaagg

SEQ ID NO: 404 - DNA, 60 bp, Eucalyptus camaldulensis
gaagtaatagttattgtaactcgaactgcctagttcaagtcttcaattgaaactcctctc

SEQ ID NO: 405 - DNA, 60 bp, Eucalyptus camaldulensis
agatcggcatatcaaggaagaaacaacgttgccaatcggaatgaccgtgaaggagtctat

SEQ ID NO: 406 - DNA, 60 bp, Eucalyptus camaldulensis
tggagcagctgtacagagaagtgccgcggatggcgatttaagattgccactgcaagtcgg

SEQ ID NO: 407 - DNA, 60 bp, Eucalyptus camaldulensis
tacattagcatttgatgtgcctacaattaactaatttaggaagggaataaaccccccaaa

SEQ ID NO: 408 - DNA, 60 bp, Eucalyptus camaldulensis
attatgttgtatccagtgacagactcgcccactattcttgaggtgctctctgcgagtata

SEQ ID NO: 409 - DNA, 60 bp, Eucalyptus camaldulensis
ttcctgggtttgtttattcaattcatggaaatgaaggaatgtgcagttgcttgctcttct

SEQ ID NO: 410 - DNA, 60 bp, Eucalyptus camaldulensis
acttctgatctctcacgacatgtttccctcaagatgatggagtgaaccgtgatgtagcta

SEQ ID NO: 411 - DNA, 60 bp, Eucalyptus camaldulensis
cccacttgttactagaagatgctagttagactataagtaatttctgtacaaacggacttg

SEQ ID NO: 412 - DNA, 60 bp, Eucalyptus camaldulensis
cagttcaggacttggcatttgccattgctcgattcatccaaaaaggaggatccttcatca

SEQ ID NO: 413 - DNA, 60 bp, Eucalyptus camaldulensis
cttgacagatactaactagactacacctcgtagatgtacattcccctgcagcaaggacta

SEQ ID NO: 414 - DNA, 60 bp, Eucalyptus camaldulensis
ctgagaagtatgctggggtttcgtatggtctgggtgttgagaatggacaaaatggttact

SEQ ID NO: 415 - DNA, 60 bp, Eucalyptus camaldulensis
accctttgatcttctaacacctcctatattgtatccttactaatgtgaatagacccaagt

SEQ ID NO: 416 - DNA, 60 bp, Eucalyptus camaldulensis
aatgtaaacgcggaaccttcatacgagcatgggaggtatgaaatttattggtcttcgatg

SEQ ID NO: 417 - DNA, 60 bp, Eucalyptus camaldulensis
tcaccggtcctggctctctaacgtatgtatgggcgcatgaatgatgttaattggactaat

SEQ ID NO: 418 - DNA, 60 bp, Eucalyptus camaldulensis
cattatgctcgtggttaacaggccaacttcttggatgtcattagtgactcccacataatg

SEQ ID NO: 419 - DNA, 60 bp, Eucalyptus camaldulensis
aagacatttaatgatgctctgggaactaccaaaactgtcatctggaacggacccatggga

SEQ ID NO: 420 - DNA, 60 bp, Eucalyptus camaldulensis
tggtctggctgatgtgaacggaatcaatttcaaatcatgcatacaggaaacatagtgttt

SEQ ID NO: 421 - DNA, 60 bp, Eucalyptus camaldulensis
ctatttgtgtcagtttttagtcagcaaaattcccaaattatgggtggaaatcaacttgtg

SEQ ID NO: 422 - DNA, 60 bp, Eucalyptus camaldulensis
ccagaggatgaacaatctgcctttgtccatgaagtatgaattaccaacaatcaaatgaaa

SEQ ID NO: 423 - DNA, 60 bp, Eucalyptus camaldulensis
ttgtacaaacactgaatgtacactggaatgttgggcaagaaaatctagtctgctggtcag

SEQ ID NO: 424 - DNA, 60 bp, Eucalyptus camaldulensis
tggtacgagaaagaaagagttttccctgtatttcttgaacaattaacattagaatgtgcg

SEQ ID NO: 425 - DNA, 60 bp, Eucalyptus camaldulensis
gactcgttatagtcaaaataagtctgtatcatatgtcccctgtatcataaatgtcaacac

SEQ ID NO: 426 - DNA, 60 bp, Eucalyptus camaldulensis
gacaagaaggacaagaaaaagaagcaatctcgccgaccatctgggagtttggatagatgt

SEQ ID NO: 427 - DNA, 60 bp, Eucalyptus camaldulensis
catcagagtgtgacagtttcttattttggggtcagatggtgtgtaagatttgtatggtct

SEQ ID NO: 428 - DNA, 60 bp, Eucalyptus camaldulensis
cctgaaggggtgaagttactctgtgattgaagttcaaacacatacaacatgtgccgccat

SEQ ID NO: 429 - DNA, 60 bp, Eucalyptus camaldulensis
gatcaggctaaaaatactgggttcttgatcggtgctgtaaagaaggccaatgcatcgtct

SEQ ID NO: 430 - DNA, 60 bp, Eucalyptus camaldulensis
caagtcgtgaaagcgatgtaatgacttctgtgatatggaatatatgagtagtaatgagta

SEQ ID NO: 431 - DNA, 60 bp, Eucalyptus camaldulensis
cttgccggaagtggaatgttcaccaagcaagaggttgatgagattgtcgagataggatat

SEQ ID NO: 432 - DNA, 60 bp, Eucalyptus camaldulensis
tccactaatcttcctggtcccagtaaaatggacatgatgtttgagtctcttcatggccca

SEQ ID NO: 433 - DNA, 60 bp, Eucalyptus camaldulensis
taagtgttggatgttccagctatttggcgaggctcgtttgcttgtgaatcagcttgagtt

SEQ ID NO: 434 - DNA, 60 bp, Eucalyptus camaldulensis
gctaggttgatctcaaaaagctaaacttaaaactatgaattgtaccagagtcgattctgt

SEQ ID NO: 435 - DNA, 60 bp, Eucalyptus camaldulensis
ccttcaatggctgggttggactactgggaccgtgaatttgctggggatgagtggtgaatc

SEQ ID NO: 436 - DNA, 60 bp, Eucalyptus camaldulensis
ttaattttcatatatatgcagacaatgtttgacaggacttgctatgggacttctctgttg

SEQ ID NO: 437 - DNA, 60 bp, Eucalyptus camaldulensis
tgatatatctagaaagaaccaactatggagagcaggggctggtcctatttggcctaatga

SEQ ID NO: 438 - DNA, 60 bp, Eucalyptus camaldulensis
acgtgcccttcacaataatggcatcgatgtgcacgtcatagtcgaaactcagaaacacat

SEQ ID NO: 439 - DNA, 60 bp, Eucalyptus camaldulensis
gaggccctgtagatacaagcagtgttgttcggaaagtttgcaagattgccgagcaaattt

SEQ ID NO: 440 - DNA, 60 bp, Eucalyptus camaldulensis
caaacatggagcgtccatcgctttggtaccatgcacattaccagtacatcaatacactaa

SEQ ID NO: 441 - DNA, 60 bp, Eucalyptus camaldulensis
tcaaactttgtggatttccatgcctattcattccgctattgtctgacagaggagaaacag

SEQ ID NO: 442 - DNA, 60 bp, Eucalyptus camaldulensis
tgtaatgggaactaaatttacccgtgatgtatctcttacttttgttgccgttgtgaagcg

SEQ ID NO: 443 - DNA, 60 bp, Eucalyptus camaldulensis
agcattctctctctctctctctctaaagcataagttaaggaaccaatgtgaaatgtcaat

SEQ ID NO: 444 - DNA, 60 bp, Eucalyptus camaldulensis
actcctagtgtcttccgttttctgctcatttgttcagtaatccgatattatagttttcgc

SEQ ID NO: 445 - DNA, 60 bp, Eucalyptus camaldulensis
aatcccggtattgtaagggtcattcattgatcgatttaatgtaatgtgaacgaatttagc

SEQ ID NO: 446 - DNA, 60 bp, Eucalyptus camaldulensis
atggacccctcttggtagtaataaattggcttttttagctgtctttcgttatttatttgc

SEQ ID NO: 447 - DNA, 60 bp, Eucalyptus camaldulensis
gtcaccggttttaatcagcaacttcttacatgcaaaacatactcggataaggaaggcctc

SEQ ID NO: 448 - DNA, 60 bp, Eucalyptus camaldulensis
ccgcatgtgatggtagtgtgtaaatttcattgttagtagccaattaggttgttgattttc

SEQ ID NO: 449 - DNA, 60 bp, Eucalyptus camaldulensis
atggcgtggaatccaactaataactggtaaaaacaggactgagaagtgattgtcgaggag

SEQ ID NO: 450 - DNA, 60 bp, Eucalyptus camaldulensis
cactgtttattcgacatccagtcttgaaagattggtttgtatagaagtacatcgatatca

SEQ ID NO: 451 - DNA, 60 bp, Eucalyptus camaldulensis
tccgaatgtgatgtcctgtgtctcttatgctacagacaagaacatgaatcatatgaatct

SEQ ID NO: 452 - DNA, 60 bp, Eucalyptus camaldulensis
aagagcctctctctctacatagaaattgaaatgagaaaaggatgcgaatgccgcgttgca

SEQ ID NO: 453 - DNA, 60 bp, Eucalyptus camaldulensis
tgctagtcagccagtctctttctaggatgttttaaaaggagctgggagtgttgtttttct

SEQ ID NO: 454 - DNA, 60 bp, Eucalyptus camaldulensis
gatgggaaagttcaataacaggaatgttgccaaatgtactgctcggatgggacagtgctt

SEQ ID NO: 455 - DNA, 60 bp, Eucalyptus camaldulensis
ttgtatttgattatacgtccccagagagacgttgagatcacattcgggcatacctttcgg

SEQ ID NO: 456 - DNA, 60 bp, Eucalyptus camaldulensis
actatgtataatttattatgcggtggatattatcaaaaaggtcatttgtgtaggaaaaaa

SEQ ID NO: 457 - DNA, 60 bp, Eucalyptus camaldulensis
aggttttatttcacgtagctctggccatcttcaagatgaaggaggatgagctgcttctaa

SEQ ID NO: 458 - DNA, 60 bp, Eucalyptus camaldulensis
accctccgtgttgctttgtatctaagtggtggttctttttgtgctaaacttgtcggtttc

SEQ ID NO: 459 - DNA, 60 bp, Eucalyptus camaldulensis
ttctccttggagagtccaatgcgagatgtaatgcttaaactcgaatcccaaatctcaacg

SEQ ID NO: 460 - DNA, 60 bp, Eucalyptus camaldulensis
tattccatgttttgattgcaaagttcgtatttcgtaggaatttttcttaatcagggtgcc

SEQ ID NO: 461 - DNA, 60 bp, Eucalyptus camaldulensis
tcaagtgtctctagagagcaggacagtcaatttgttgccttgctaagaaagacactgaaa

SEQ ID NO: 462 - DNA, 60 bp, Eucalyptus camaldulensis
tctgtccattcacttggtaagatattgcgctaacttatctatccatgcttccaatcggca

SEQ ID NO: 463 - DNA, 60 bp, Eucalyptus camaldulensis
ctactttaatgtgttttgccttggctaggacctctttgatagactgttattgttatggtg

SEQ ID NO: 464 - DNA, 60 bp, Eucalyptus camaldulensis
aagggcattctctcaccttaggggccaacttgttatgcttccacctgtggatttgattaa

SEQ ID NO: 465 - DNA, 60 bp, Eucalyptus camaldulensis
catctcgagcatcagggaaacaagacttggaaggattaatgaaagaggttcaagaggcca

SEQ ID NO: 466 - DNA, 60 bp, Eucalyptus camaldulensis
tggcagtgcatgccaacgttgcagctaagtcacttgagcaaaactgttctctgaatgtta

SEQ ID NO: 467 - DNA, 60 bp, Eucalyptus camaldulensis
tcggcttctccccatggaagtcggattcatagtattgcagaataaagaaccgttcttgtt

SEQ ID NO: 468 - DNA, 60 bp, Eucalyptus camaldulensis
gcaaatactttgtacttggattatcttgtgcaagcttatctcaagtccaattcccaaaaa

SEQ ID NO: 469 - DNA, 60 bp, Eucalyptus camaldulensis
catggattttattccttctacaattacctcgaaaaacagatgaagacagttctcgtgcat

SEQ ID NO: 470 - DNA, 60 bp, Eucalyptus camaldulensis
aggggccctggatttccgtatcttgtacaattcacatgaccactaatgataaagacctaa

SEQ ID NO: 471 - DNA, 60 bp, Eucalyptus camaldulensis
tttgcttttcgtgtcagcgtgaagaagccgaggcagactctttggtaaatattcagttgt

SEQ ID NO: 472 - DNA, 60 bp, Eucalyptus camaldulensis
attgggttggataaatatccaagcctgattgcagaaagaaatttaaggaagggagcttca

SEQ ID NO: 473 - DNA, 60 bp, Eucalyptus camaldulensis
aggaaaaacaaacttcacgagagagaagagagagacaaaagacgcctgccaagaagttct

SEQ ID NO: 474 - DNA, 60 bp, Eucalyptus camaldulensis
gggctgctgtgctagtgcagcttgctgtttgtggggttattgttgttcatctcattttat

SEQ ID NO: 475 - DNA, 60 bp, Eucalyptus camaldulensis
tggtttgaaatgacatgccggagcagaaaacatcactttgtgtgctgaaatcaaggttct

SEQ ID NO: 476 - DNA, 60 bp, Eucalyptus camaldulensis
tcttttgtcggatggtggagaactatggttaatcccttctgtaagaagattttgatgagg

SEQ ID NO: 477 - DNA, 60 bp, Eucalyptus camaldulensis
cactcttggtctgttttgtgcattaataagacttatatctgtgttttgtggaagttcgcc

SEQ ID NO: 478 - DNA, 60 bp, Eucalyptus camaldulensis
gttggccataaaggctgtaacatcttgaacattcttttgtcatcgcatgtgcaaaatctt

SEQ ID NO: 479 - DNA, 60 bp, Eucalyptus camaldulensis
ttgtactacatcgtattttttctcccccattatgtacatagcaatttgcgtgccccaaaa

SEQ ID NO: 480 - DNA, 60 bp, Eucalyptus camaldulensis
cttgttcttacagcgaataagacagttgttggtaatagaaagtagagattcctggtacca

SEQ ID NO: 481 - DNA, 60 bp, Eucalyptus camaldulensis
cctgctgtcttgtagcttctgtcataagtaacttcaagctcaagaaaagcacttgtcaga

SEQ ID NO: 482 - DNA, 60 bp, Eucalyptus camaldulensis
tctctctctctctctctctctctcttttttaaacatattttaacaagatgatggctccgt

SEQ ID NO: 483 - DNA, 60 bp, Eucalyptus camaldulensis
tggagacaatgaagtccagcgtacaatgcttgagattgtgaaccagcttgatggctttga

SEQ ID NO: 484 - DNA, 60 bp, Eucalyptus camaldulensis
gttgtaaccacagaagtcggcgatccaataatttcatccgtcttctacttagaacatcta

SEQ ID NO: 485 - DNA, 60 bp, Eucalyptus camaldulensis
atgaagtttggtcatgaaatacattccatcgacctgtcgtccatatctatcaggagatgc

SEQ ID NO: 486 - DNA, 60 bp, Eucalyptus camaldulensis
ttccaattctcgttgtctctaggtccgagagttaagttgcattgccatctctccatgcta

SEQ ID NO: 487 - DNA, 60 bp, Eucalyptus camaldulensis
aggatatctgtgtggccagtacttcctcagatgtaaaggtcaatggcttcgtatgcaaga

SEQ ID NO: 488 - DNA, 60 bp, Eucalyptus camaldulensis
tcaacgttgagtgcctatgtacatccaagtcatgtacttatccagatcgggtagtgaaag

SEQ ID NO: 489 - DNA, 60 bp, Eucalyptus camaldulensis
ctacttccgcgacctgcgcagtgactcatccaagcccggcgaagaagaaaagaccatgaa

SEQ ID NO: 490 - DNA, 60 bp, Eucalyptus camaldulensis
ctcaaccagtgatgggagcttgtctgttgctatctcattgcaaacagagcacatgaaact

SEQ ID NO: 491 - DNA, 60 bp, Eucalyptus camaldulensis
cagattttgccgacgcaatccggttggattaacacatcaatttgttgtatttagattaat

SEQ ID NO: 492 - DNA, 60 bp, Eucalyptus camaldulensis
agatgttcctggaggagtgcatttgaactttttgaaggcagaaagaagcttgcacagatg

SEQ ID NO: 493 - DNA, 60 bp, Eucalyptus camaldulensis
ttcattacacacgaggttcctttctcggagatcaacaaggccttcgactacatgcttcaa

SEQ ID NO: 494 - DNA, 60 bp, Eucalyptus camaldulensis
agaagccagataactaggtcatgaacctgattttctctgtccgacactggcaacagaaaa

SEQ ID NO: 495 - DNA, 60 bp, Eucalyptus camaldulensis
tggctcagtggaatgtcctgcagttcaacacaggctcaacaactccattcatcatcaaat

SEQ ID NO: 496 - DNA, 60 bp, Eucalyptus camaldulensis
atgtcttcggacgtccaggcattgccgcgtaccgtttgcatttgtctttatgatgaataa

SEQ ID NO: 497 - DNA, 60 bp, Eucalyptus camaldulensis
ggaggccaaaatccttcattactgtgtctgctgagtatgactcaaaggcaatcaatgcag

SEQ ID NO: 498 - DNA, 60 bp, Eucalyptus camaldulensis
tctttgctcgacaactttgagtggaggttgggatacacatcacggttcggaattgtctat

SEQ ID NO: 499 - DNA, 60 bp, Eucalyptus camaldulensis
tctccctctttgagaagtagaggcaatcgcgatgatactgctactgcagtttgcgtaaat

SEQ ID NO: 500 - DNA, 60 bp, Eucalyptus camaldulensis
ccttcccgagtgaatgaattagttctgcaatggtatcaaaaggattctcctagataaaaa

SEQ ID NO: 501 - DNA, 60 bp, Eucalyptus camaldulensis
ccgagttgtatcgtttcgattgagagaaatacgtgaattgagtggagtcgagggaaaaaa

SEQ ID NO: 502 - DNA, 60 bp, Eucalyptus camaldulensis
ctcaccatgcaagcggtaataatgtaacatcaacttgatcgagtttatgaaggaaagctc

SEQ ID NO: 503 - DNA, 60 bp, Eucalyptus camaldulensis
caagctctcatgattcaaccgaattacatcaaggcacttcttcggagagctgtctccaaa

SEQ ID NO: 504 - DNA, 60 bp, Eucalyptus camaldulensis
ggaaataggcagacggggtcctttgtacatctctctttaaggaaattaattcgattaatt

SEQ ID NO: 505 - DNA, 60 bp, Eucalyptus camaldulensis
atcgagtatgttttcttgtgaaattgtgagtctctgaaccttctgtaaatgcgtttgctg

SEQ ID NO: 506 - DNA, 60 bp, Eucalyptus camaldulensis
gtacgatggagaacagtagtagcaatagactagcgagagggtcgatcattttggtggata

SEQ ID NO: 507 - DNA, 60 bp, Eucalyptus camaldulensis
gtattccttaacagacgctaaaaactcgtctatcgacttgagactgtcttggcgtgtatt

SEQ ID NO: 508 - DNA, 60 bp, Eucalyptus camaldulensis
attatggccgggggtgctggttgttagattcttacccgggtgatttgtgctttgaagaca

SEQ ID NO: 509 - DNA, 60 bp, Eucalyptus camaldulensis
tgagtcgatatccttgaaaagcaatttgagggtactcaactcaggtgccagaagcttgaa

SEQ ID NO: 510 - DNA, 60 bp, Eucalyptus camaldulensis
aaatgtctctccttttgtttcttccttttgggtatttgtgacaatcgttttgcgattcga

SEQ ID NO: 511 - DNA, 60 bp, Eucalyptus camaldulensis
gactctggacgagtaactgagtttgttttaactgtaaagttgatcacgactccttagggc

SEQ ID NO: 512 - DNA, 60 bp, Eucalyptus camaldulensis
atgcacatgtcaggtgaagagtgtcgtagggcatgtcaaaatctagatcgaaccaagctt

SEQ ID NO: 513 - DNA, 60 bp, Eucalyptus camaldulensis
tataaggtgtcttgcactggagaaagtggcagaatcacttgagctgccttgcaagtactt

SEQ ID NO: 514 - DNA, 60 bp, Eucalyptus camaldulensis
gctgagtttaagtctggggatatgagactatgtcctgtttcatatttctcgatctatgca

SEQ ID NO: 515 - DNA, 60 bp, Eucalyptus camaldulensis
ttctttgaaaaatcaaatgcaatttccagaggttcaatggagcattaaacctcgagctaa

SEQ ID NO: 516 - DNA, 60 bp, Eucalyptus camaldulensis
attgaggccatcgatactcacagcgggtacgaattgccttggagtcttacaaagtacatt

SEQ ID NO: 517 - DNA, 60 bp, Eucalyptus camaldulensis
cagccattttgccttaccaaatattgtgaataacttcaggacgagaactgatgcatgatg

SEQ ID NO: 518 - DNA, 60 bp, Eucalyptus camaldulensis
cactgcttatgtacatattgcttatgtacatattgacacaagttcttcaaataatgcccg

SEQ ID NO: 519 - DNA, 60 bp, Eucalyptus camaldulensis
aagccacggttgtgacataaagatatgtttgtagcagtttgttctaccttgtgtaataaa

SEQ ID NO: 520 - DNA, 60 bp, Eucalyptus camaldulensis
ttttttcttgggttaattttttgtaatgccgcactaataataattgtatgacgccgctgc

SEQ ID NO: 521 - DNA, 60 bp, Eucalyptus camaldulensis
cctgttattaaggggaacaaatggtcatctactaagtggctgcgtgtgcacgagtacaaa

SEQ ID NO: 522 - DNA, 60 bp, Eucalyptus camaldulensis
tgtacatcttgtattgcatagggaaaatgcttgagcctagcgttccagacaatgtaagta

SEQ ID NO: 523 - DNA, 60 bp, Eucalyptus camaldulensis
cgtttggtttgaactttgcaataaaagtagccatgagatctgttttgatcttaaaaaaaa

SEQ ID NO: 524 - DNA, 60 bp, Eucalyptus camaldulensis
gtgctaagttgctcttggaatcattggaagaatcggtgcattgacaaatcttcttgaaaa

SEQ ID NO: 525 - DNA, 60 bp, Eucalyptus camaldulensis
tgacatctcatctctacaaatatttactccacatgaactggactatctgctctgtggccg

SEQ ID NO: 526 - DNA, 60 bp, Eucalyptus camaldulensis
atacaccaatcttggatgagctagcggagccaactatagcacaagaagggacagaatcaa

SEQ ID NO: 527 - DNA, 60 bp, Eucalyptus camaldulensis
agtaattggatatggaaaagttacttttgtcactgttcatgcttgcttgcggctattaat

SEQ ID NO: 528 - DNA, 60 bp, Eucalyptus camaldulensis
ttcatctcttattggtggagatgcgcatataatgaagaaacccattattgtggccatgat

SEQ ID NO: 529 - DNA, 60 bp, Eucalyptus camaldulensis
gaattgaagctggacaaaagctggattcgaaggccaacaaagcaggctaatgaagagagt

SEQ ID NO: 530 - DNA, 60 bp, Eucalyptus camaldulensis
gtttgtttggacagggaactgaaataaataatcttgctgcttgctcaatatcgatgaaat

SEQ ID NO: 531 - DNA, 60 bp, Eucalyptus camaldulensis
agagacttttgatgatgaaatgagtgatgatgcagaaagcaaggaagaaactgcagatgc

SEQ ID NO: 532 - DNA, 60 bp, Eucalyptus camaldulensis
acatcatcagacgacccagtacaaggtcatattttagcaaagaacgaagccacaatagtg

SEQ ID NO: 533 - DNA, 60 bp, Eucalyptus camaldulensis
ggaaaggggagctgtactgcttctcttgccttttgatcttttgcggttaaattggaagga

SEQ ID NO: 534 - DNA, 60 bp, Eucalyptus camaldulensis
accgaatgatgctcactttctcagtggtttccatcaccaaaggtatcagacacagtggtt

SEQ ID NO: 535 - DNA, 60 bp, Eucalyptus camaldulensis
tctggagctggcatttcatcatacgttgtatttattgctgctatgttccctgccaaaata

SEQ ID NO: 536 - DNA, 60 bp, Eucalyptus camaldulensis
catgtaatcaccatgagaaatgtgagctggatgacgagttcttcttcgtaattctttcaa

SEQ ID NO: 537 - DNA, 60 bp, Eucalyptus camaldulensis
caaaacaacgagaatgcagttcagtcttggcatctagatggttacgatttctgggttgtc

SEQ ID NO: 538 - DNA, 60 bp, Eucalyptus camaldulensis
tctggttcacaggccagtttgtggtgcaatatccgaagtgtgtgctgcatatgtaagaat

SEQ ID NO: 539 - DNA, 60 bp, Eucalyptus camaldulensis
tcgattgggcatcacaatcagagtttaagattgcaaattcaggtcatcagccaacccact

SEQ ID NO: 540 - DNA, 60 bp, Eucalyptus camaldulensis
catttccttccctaaagagtttctcttgtgagagtgttactagagctatgcgattctgat

SEQ ID NO: 541 - DNA, 60 bp, Eucalyptus camaldulensis
aaaggtccagcagtcgtcattgatgggactctttatgtgtcggatcaaagttcaggacta

SEQ ID NO: 542 - DNA, 60 bp, Eucalyptus camaldulensis
gaaaatgaatccttggaaaatcacagggattgataatgtcgaaaatgcttcaagctttga

SEQ ID NO: 543 - DNA, 60 bp, Eucalyptus camaldulensis
ttgttttccgctgggaatgtatgctttttccaagattgcaagttgtaaaatttatgttgg

SEQ ID NO: 544 - DNA, 60 bp, Eucalyptus camaldulensis
gcgtcaagcattcaccaggagatgacatcatgtgtatgtggtgtgactatcatttcttga

SEQ ID NO: 545 - DNA, 60 bp, Eucalyptus camaldulensis
gcagagtacaacaatctaagccaatatgtatgacagatcattctttgagcaattcagcgc

SEQ ID NO: 546 - DNA, 60 bp, Eucalyptus camaldulensis
tgtgcttgtggagaagcaagaaggtcggccctcaattcaatcactgttcagaacttgata

SEQ ID NO: 547 - DNA, 60 bp, Eucalyptus camaldulensis
gtgagggatgtaatcagcacactttcagtgtataaagagatttccgatggatgttcactt

SEQ ID NO: 548 - DNA, 60 bp, Eucalyptus camaldulensis
ttcatgtttcaccttctagagatgtggatgagctcaatactcctgaatggctggacgact

SEQ ID NO: 549 - DNA, 60 bp, Eucalyptus camaldulensis
ttctggaccctctcgtgctttgtgcaacaactacttaagaaataaagagaaacccattca

SEQ ID NO: 550 - DNA, 60 bp, Eucalyptus camaldulensis
agattccaatcctcagattagactgacagaattttgatattcttggagaattctgttcgg

SEQ ID NO: 551 - DNA, 60 bp, Eucalyptus camaldulensis
ttgttgtgctttttcctgctcttcaattatcaagcatgtgaagaaagctaggcgagctca

SEQ ID NO: 552 - DNA, 60 bp, Eucalyptus camaldulensis
gcgaattaacggaggcagctcaagcaaagtactacagaaaagaaaacataagcttgccgc

SEQ ID NO: 553 - DNA, 60 bp, Eucalyptus camaldulensis
tgtagactgccaatgtcaaatggatatcttgcaaaggcttcatcttatgcagtaatatat

SEQ ID NO: 554 - DNA, 60 bp, Eucalyptus camaldulensis
aacctataagtacattactctcctctactctctaaacatgtacagtgaaagtatgaactg

SEQ ID NO: 555 - DNA, 60 bp, Eucalyptus camaldulensis
cgacttcttttgctgggacctgccctgtccattaactgatattggtttctaaggttatgg

SEQ ID NO: 556 - DNA, 60 bp, Eucalyptus camaldulensis
gcctgcttcatcaaatacccatgtggggcttaagagctatatattcttcagatttccctt

SEQ ID NO: 557 - DNA, 60 bp, Eucalyptus camaldulensis
tagtgctgatttttagattatcctgatttgcctgctattataaaacactgcttggactca

SEQ ID NO: 558 - DNA, 60 bp, Eucalyptus camaldulensis
tgctgtcaagtttctgttatccttcttatctattcttctttacccaattggcgtgcttgg

SEQ ID NO: 559 - DNA, 60 bp, Eucalyptus camaldulensis
tttgtcaagaccctgaccggcaagaccattaccctcgaggttgagagctccgacaccatt

SEQ ID NO: 560 - DNA, 60 bp, Eucalyptus camaldulensis
aacacgccaccttggttgggagaaattcttctaaatagtacaaagtgctgtagtgcagat

SEQ ID NO: 561 - DNA, 60 bp, Eucalyptus camaldulensis
tcgtgcatatatgaagcattgcaatgtgtatatgaatccatgtgaagttttgccttgaat

SEQ ID NO: 562 - DNA, 60 bp, Eucalyptus camaldulensis
tcacactttcttgtaaagctatgatctgatttcacgtgcccatggtttatatcgaaaaag

SEQ ID NO: 563 - DNA, 60 bp, Eucalyptus camaldulensis
agaaccgacagatcattgttgttatctagagatagtactgagaaaccaggatggagaagg

SEQ ID NO: 564 - DNA, 60 bp, Eucalyptus camaldulensis
ccatgaatcagcgggcaagaacctcacctaatgttgtgatgaattagcgattacggtcaa

SEQ ID NO: 565 - DNA, 60 bp, Eucalyptus camaldulensis
acaattcgattcagtcggtcatgtacagcatccgatcttcaggatttggtggttactctt

SEQ ID NO: 566 - DNA, 60 bp, Eucalyptus camaldulensis
gcataaggcgtctgatagagaccctccttgatgcttcgaaattggattcgactgcaaata

SEQ ID NO: 567 - DNA, 60 bp, Eucalyptus camaldulensis
tgatgaaaactgtgaagattcaggatatagatagcttgcaagatgagatgatggacctga

SEQ ID NO: 568 - DNA, 60 bp, Eucalyptus camaldulensis
gggactctcgacatccagtgttcgccgtggatggattggtttcacggcggattgcagttt

SEQ ID NO: 569 - DNA, 60 bp, Eucalyptus camaldulensis
tcttgactttgatatttctatgtattaacgaaccgcatttaatgagagaaaaaacctttc

SEQ ID NO: 570 - DNA, 60 bp, Eucalyptus camaldulensis
acccaaaggctataaatgaagtttcatggttcgggattgagcaagaaaacagcctactgc

SEQ ID NO: 571 - DNA, 60 bp, Eucalyptus camaldulensis
ttttgcttcagtggcttcttggtaccaagtattccatattttttacactctgaagaattg

SEQ ID NO: 572 - DNA, 60 bp, Eucalyptus camaldulensis
atgccctctgttcgttttgttgaatatattaaaatttgggaatttggggactgaattctg

SEQ ID NO: 573 - DNA, 60 bp, Eucalyptus camaldulensis
cgtatatggatgtgcctgaatgatggaaattgtgatgttctgtcttctttttcagtaaat

SEQ ID NO: 574 - DNA, 60 bp, Eucalyptus camaldulensis
ctactccttaaccgtcttaggagttatactctacagtgaagccaaaaagaggagcaaatc

SEQ ID NO: 575 - DNA, 60 bp, Eucalyptus camaldulensis
gcattgtaagatatgtgataatgtagaattcagctgtaactgcttagaaacgtgtttgtc

SEQ ID NO: 576 - DNA, 60 bp, Eucalyptus camaldulensis
tcccgatcgtctggggcttgtgaatcaatccttaagtttcaattgaataaaagagtcttt

SEQ ID NO: 577 - DNA, 60 bp, Eucalyptus camaldulensis
tttgtctttatgatgaataaagtcgacgactgctcgacaacttagcgtgttgcaactggg

SEQ ID NO: 578 - DNA, 60 bp, Eucalyptus camaldulensis
caagatttgtatttcaagaagactgtcaaatatgtgggagagccaatgagccacttagaa

SEQ ID NO: 579 - DNA, 60 bp, Eucalyptus camaldulensis
aatgcaattccaggacagacgtctatgcctatggaatactcctgctcgagatactaatcg

SEQ ID NO: 580 - DNA, 60 bp, Eucalyptus camaldulensis
cttgtcactcaataagccagggaatgactggacgtgtatattttttgctttggttctggg

SEQ ID NO: 581 - DNA, 60 bp, Eucalyptus camaldulensis
agctgtttcaatgatgctcggatgttggacttaatgtggtgcttttcacccttcatataa

SEQ ID NO: 582 - DNA, 60 bp, Eucalyptus camaldulensis
gacggtgaattttcctttctatattgagttttgacagatttacagtatgtaatgaggagc

SEQ ID NO: 583 - DNA, 60 bp, Eucalyptus camaldulensis
gagggatttatgtggaaaacggggctgcttttgtcgaggtcttctgggtttccttatata

SEQ ID NO: 584 - DNA, 60 bp, Eucalyptus camaldulensis
ttagggtttagtgaaagtaggactcaaagtttggactgagcaaaggagttggatgtgtga

SEQ ID NO: 585 - DNA, 60 bp, Eucalyptus camaldulensis
ctttccaggaagttttgactcgactggattatccctcaggttaagtcgcttctcatgcaa

SEQ ID NO: 586 - DNA, 60 bp, Eucalyptus camaldulensis
ttgtaattagtcactgttggttctactttcattttcttatctaaagtttctcaaatggca

SEQ ID NO: 587 - DNA, 60 bp, Eucalyptus camaldulensis
ttcttctgggactccacctctaatgtctcgaccttcattaagggatatacagatgcagca

SEQ ID NO: 588 - DNA, 60 bp, Eucalyptus camaldulensis
ctgctacatcggactggatttcatctttgcttcggagagagcaataaggatttgccgaat

SEQ ID NO: 589 - DNA, 60 bp, Eucalyptus camaldulensis
aggctctccaactggaatgagtgtaaatagtgtaaagcatgatgtgaaatggtaagctgt

SEQ ID NO: 590 - DNA, 60 bp, Eucalyptus camaldulensis
tgtcgatctattggcataggaagtggaggcttcctaataggtcgagttcgtcaacaaagt

SEQ ID NO: 591 - DNA, 60 bp, Eucalyptus camaldulensis
tttctctttcttttactcacatgctgataattgtccatctgtgggagagatcttataaag

SEQ ID NO: 592 - DNA, 60 bp, Eucalyptus camaldulensis
attggactgatcgagtgtaatccaaagatcaacgattctggtctgagtcaaacgttgaag

SEQ ID NO: 593 - DNA, 60 bp, Eucalyptus camaldulensis
tggagtatgctgtttgactgatacaaatttgaataccagtgaagcaggcaagaatttggt

SEQ ID NO: 594 - DNA, 60 bp, Eucalyptus camaldulensis
atgctcgtgccagcacagtccgctgaggtgaattataataaaggattatgacggtggaat

SEQ ID NO: 595 - DNA, 60 bp, Eucalyptus camaldulensis
agcttctcattagttggtttgatgaaatgagaatatgcagttggccctggaaaacacaca

SEQ ID NO: 596 - DNA, 60 bp, Eucalyptus camaldulensis
gtagatctatccatcatcggttttaccttaagatcattagaaatggaggcttcctttgta

SEQ ID NO: 597 - DNA, 60 bp, Eucalyptus camaldulensis
ataatgaacttcatcctgtattaatgatggagatggtgaggtttatggtccctttgagat

SEQ ID NO: 598 - DNA, 60 bp, Eucalyptus camaldulensis
caacgtggagacgtgggtgagcgccgcactgaccgacgagaacacctgcatggatggttt

SEQ ID NO: 599 - DNA, 60 bp, Eucalyptus camaldulensis
aatatgacaattgacagctatagtttactggaggtttatgattcctaaggttgattaccg

SEQ ID NO: 600 - DNA, 60 bp, Eucalyptus camaldulensis
atgttgaatagagctgcattaatatggagggttctcgctatttggagcaaaagtatcggt

SEQ ID NO: 601 - DNA, 60 bp, Eucalyptus camaldulensis
ctcgaactggttgtgcatttctaagattgtattatgttgagcatgggcaatatgtaatgt

SEQ ID NO: 602 - DNA, 60 bp, Eucalyptus camaldulensis
atttgagacagtttccttctttatgaggacctgtgtaatgaagtagggtaattacgatat

SEQ ID NO: 603 - DNA, 60 bp, Eucalyptus camaldulensis
ggttggagaatctggtggggcatacaatagtggcggtcgtcacatatctaacacatttat

SEQ ID NO: 604 - DNA, 60 bp, Eucalyptus camaldulensis
cgacctttttgtattttggtcacggtctgaaggaaatcaagtgctaagaaaccgggggat

SEQ ID NO: 605 - DNA, 60 bp, Eucalyptus camaldulensis
tgttgtatcttctcttatatgcaatattgcaagcaatgtcaatccggtgtgtgactggtc

SEQ ID NO: 606 - DNA, 60 bp, Eucalyptus camaldulensis
gtgattgaggctgtcgtctgtgtagatatggatcatataagagttgtgaattgggatcgt

SEQ ID NO: 607 - DNA, 60 bp, Eucalyptus camaldulensis
taaatgggaataatgcagtattagtgttcctacttgaaataatgagcatggttctgcgca

SEQ ID NO: 608 - DNA, 60 bp, Eucalyptus camaldulensis
ccacaatctccaagcagatgtagttgctgccgatgtggtcgagttcaatcctcagcgtga

SEQ ID NO: 609 - DNA, 60 bp, Eucalyptus camaldulensis
gagagctaatacgcacattttctctttgtacttaaaacggaaggtctccaagctggaatt

SEQ ID NO: 610 - DNA, 60 bp, Eucalyptus camaldulensis
gtttcttcctttgggtattgtgacaatcgtttgcgattcgaatgtaataaatatcttctc

SEQ ID NO: 611 - DNA, 60 bp, Eucalyptus camaldulensis
acgagattattactgggtgggatgggtataggagtattgttcttcctgctatctaaatgg

SEQ ID NO: 612 - DNA, 60 bp, Eucalyptus camaldulensis
gtgttttccctttaccggttcgatagcagtatcgatcatttatgggaaatgaaaaatata

SEQ ID NO: 613 - DNA, 60 bp, Eucalyptus camaldulensis
gcgataggaagacactgttacaatcaatttgatcgctatgtaagacactgttattcatcg

SEQ ID NO: 614 - DNA, 60 bp, Eucalyptus camaldulensis
tcatatatacatttttgaatagtgagcattatgcgacctcgctgtccttacacagtgcag

SEQ ID NO: 615 - DNA, 60 bp, Eucalyptus camaldulensis
ttgttcttaactggaagcaaagatggtgatgtgaaactttgggatgccaaaagtgccaag

SEQ ID NO: 616 - DNA, 60 bp, Eucalyptus camaldulensis
tagttgaagggcaagcgtttctagttcttatccttttaccatgcttaatagagtcagtgt

SEQ ID NO: 617 - DNA, 60 bp, Eucalyptus camaldulensis
aaagacagaaacttatgatgactctgaagtgtgtccggtcgtccagaaaggggtgctaat

SEQ ID NO: 618 - DNA, 60 bp, Eucalyptus camaldulensis
accagggcaatatggttcaatgatttcttgaacactatcttcttctcgtccccaactgtc

SEQ ID NO: 619 - DNA, 60 bp, Eucalyptus camaldulensis
gggtgtggaaccatatgatatagaagatgctgatgcaagctgcaaaggccttgtttcgta

SEQ ID NO: 620 - DNA, 60 bp, Eucalyptus camaldulensis
agctggggaagataatgcagaagggttcatcgagctctcgtttcggaggagctcacgtgg

SEQ ID NO: 621 - DNA, 60 bp, Eucalyptus camaldulensis
gtctgtccacaagttcgatgattttttaaaaatcccttgtgatctaattcgttggcaggt

SEQ ID NO: 622 - DNA, 60 bp, Eucalyptus camaldulensis
cttctagatgttacttgacaattctatttaacataagctagcttctcacttccgtttacc

SEQ ID NO: 623 - DNA, 60 bp, Eucalyptus camaldulensis
atcagctgcttatcttccttctggtgagaaccttggggttttcttcatcgactggaatgt

SEQ ID NO: 624 - DNA, 60 bp, Eucalyptus camaldulensis
catcatgttctttatctgtaagaaactttaattcaatctttatgtgagcggacaatggcg

SEQ ID NO: 625 - DNA, 60 bp, Eucalyptus camaldulensis
gcctcgatatctgtcggcagattgataatgctaaagaaacgatcgaacttattgtttatt

SEQ ID NO: 626 - DNA, 60 bp, Eucalyptus camaldulensis
aagaagctggtattgtttgtgagcagaaaacctaaataatcatcttctagtcatcatcgg

SEQ ID NO: 627 - DNA, 60 bp, Eucalyptus camaldulensis
acgtcccgtggatccagagtttggggagaaggacctggtcaagctgggtatgcatcactt

SEQ ID NO: 628 - DNA, 60 bp, Eucalyptus camaldulensis
cttccctcgccggagagattcgattttatttatttaatttcgcggatttcttttgaaaaa

SEQ ID NO: 629 - DNA, 60 bp, Eucalyptus camaldulensis
actcacaaatgacgttgagaagtcgaatgaaatggtgctcatcctgcaagagaagctgga

SEQ ID NO: 630 - DNA, 60 bp, Eucalyptus camaldulensis
atagaggagagatgtgccgttggatgtaagcttgatcctttgctttctttcaagtaatca

SEQ ID NO: 631 - DNA, 60 bp, Eucalyptus camaldulensis
ggaccatgtgttttactggagattctagccagcgtcagagatggcacaagataacttcat

SEQ ID NO: 632 - DNA, 60 bp, Eucalyptus camaldulensis
acgatgacgtttaggctaccagattgattaattcattcgtttattggttttttcgttgct

SEQ ID NO: 633 - DNA, 60 bp, Eucalyptus camaldulensis
ggatgaatggtatttatgaggtttctagataataaatttcgctatgatcgtgtcaaaaaa

SEQ ID NO: 634 - DNA, 60 bp, Eucalyptus camaldulensis
ctgcaatttgttctcggttgcaactgaactacttaatttgcatgtcgataatcatattca

SEQ ID NO: 635 - DNA, 60 bp, Eucalyptus camaldulensis
ctgtctgagtgacagctctctctcttgattccaaaaattgttcttgagaataaaaagtat

SEQ ID NO: 636 - DNA, 60 bp, Eucalyptus camaldulensis
agtagctttcgttttcctacctatgggacattgtcacctcaggattccaggagggattat

SEQ ID NO: 637 - DNA, 60 bp, Eucalyptus camaldulensis
gattttgtgatctttagacattggtgatctcctgtggctggtcttcaccaaggttggagt

SEQ ID NO: 638 - DNA, 60 bp, Eucalyptus camaldulensis
gctccgtatgttttactaggtacttgtgggtgcaaaattactggagatgaagctccgtgt

SEQ ID NO: 639 - DNA, 60 bp, Eucalyptus camaldulensis
ccaatgtgctaaattctaatgtctgatgtctcatgtcatgagcatgtgacctttgcaaat

SEQ ID NO: 640 - DNA, 60 bp, Eucalyptus camaldulensis
accgacatttctcaaacacagactttttcctgtaactgaaaccagaaggaacttttcttc

SEQ ID NO: 641 - DNA, 60 bp, Eucalyptus camaldulensis
acacgatgtctgtggatattgctttgcactttaatcaggtcctctgcagagtttcctgaa

SEQ ID NO: 642 - DNA, 60 bp, Eucalyptus camaldulensis
tcaagttcgatgtggaagagacgtggcaaaacttttccaccatgaacctacgaatgagaa

SEQ ID NO: 643 - DNA, 60 bp, Eucalyptus camaldulensis
taagtggaagaggcatgtcaatgcctacaagaaaatgaataagcttcttgacactggaag

SEQ ID NO: 644 - DNA, 60 bp, Eucalyptus camaldulensis
tcgatgttatacgtgaccacaagcatagtgacataaaagttcaagagcttactattttgt

SEQ ID NO: 645 - DNA, 60 bp, Eucalyptus camaldulensis
agaaggagcaatttgtgcataaatctttccactttgactctgagaagctggcagcagtta

SEQ ID NO: 646 - DNA, 60 bp, Eucalyptus camaldulensis
ctggagcattacttaaatatattcaggaattgtgcttcttccgaatacagattgcaagga

SEQ ID NO: 647 - DNA, 60 bp, Eucalyptus camaldulensis
ctttgcgtcaatatggcgcgtgaagtatgtattgaccatgcactttgcctcacgaaatta

SEQ ID NO: 648 - DNA, 60 bp, Eucalyptus camaldulensis
taagaagctcgtttcagcgaccaaggaaggtttgaagctcgatgagagtgaagatgagaa

SEQ ID NO: 649 - DNA, 60 bp, Eucalyptus camaldulensis
gacacatgatgagtttcctgggatagagtgtttggatgaggcaggcagcaaacagtttat

SEQ ID NO: 650 - DNA, 60 bp, Eucalyptus camaldulensis
gtggagccaacgaagtgcaactcggctacagcaaggggactgctctcggcgcagagatca

SEQ ID NO: 651 - DNA, 60 bp, Eucalyptus camaldulensis
gctccatttgtatgtgaggaaaatgtcaaatgaagcggagaaagttacgatccttcttct

SEQ ID NO: 652 - DNA, 60 bp, Eucalyptus camaldulensis
agttgtgcttggttgggggatctgtcaaatttggtggatatttgtatttgatcatttgag

SEQ ID NO: 653 - DNA, 60 bp, Eucalyptus camaldulensis
ttcctggtacatgatagttttgtctgcgttgatgccgacactgtccttgtaaccctacta

SEQ ID NO: 654 - DNA, 60 bp, Eucalyptus camaldulensis
attttctaactgctgggcaatacagtcagaattgtatcatttggaacctgggtggaacga

SEQ ID NO: 655 - DNA, 60 bp, Eucalyptus camaldulensis
aagcaaagttcatttgatgattgtaatcgatgagtgtaacttccatatatgcataagggg

SEQ ID NO: 656 - DNA, 60 bp, Eucalyptus camaldulensis
cctctactactattatgccagatccgacatgtctggcttcatgcaaacttcgttcttctt

SEQ ID NO: 657 - DNA, 60 bp, Eucalyptus camaldulensis
tcgagggaaagcgcattgtatacttctgtacgataaataaagaaggcaatacgagtggaa

SEQ ID NO: 658 - DNA, 60 bp, Eucalyptus camaldulensis
ttgtgtacatatgttgtgacattttcttcattcaaccagtttttagtgtttaccacaggg

SEQ ID NO: 659 - DNA, 60 bp, Eucalyptus camaldulensis
gtttggaaatggaccagcaacaattcagtgactttatgttcatctgtatgaaaaggaact

SEQ ID NO: 660 - DNA, 60 bp, Eucalyptus camaldulensis
tgagtcgaatggttctgtatattgctatgatataagttcttggttgtgatagtaagggtt

SEQ ID NO: 661 - DNA, 60 bp, Eucalyptus camaldulensis
gttcctgaacggcattaggagagtgatgtgatagttaaactaagacattaagtgtatgga

SEQ ID NO: 662 - DNA, 60 bp, Eucalyptus camaldulensis
tctggagtatcatgggactggccattatcgtgaggaactccagaagaactttgtagtgaa

SEQ ID NO: 663 - DNA, 60 bp, Eucalyptus camaldulensis
actcatcgatcgaggcgaaactagagcgtgcaaatgcactggagagagaaatcttgatta

SEQ ID NO: 664 - DNA, 60 bp, Eucalyptus camaldulensis
attggaatggattgaccaagtgatataaaagctagctgcaacaatattctacaaaccaac

SEQ ID NO: 665 - DNA, 60 bp, Eucalyptus camaldulensis
ccatttacctctacctcatttacttcttcgaaggagcatgatcaaacatggtcaggccca

SEQ ID NO: 666 - DNA, 60 bp, Eucalyptus camaldulensis
gaatgtagctggagatttgaacacaatatgagcttcttcatattttcatcgcaccagctg

SEQ ID NO: 667 - DNA, 60 bp, Eucalyptus camaldulensis
cctggacccgtcctatcacaaaaaggagtgaagtaccatgaaccagaacactggaaattt

SEQ ID NO: 668 - DNA, 60 bp, Eucalyptus camaldulensis
gatacctccctcgcatcattcggtgtggttcgattgcgtacctttggtattgttgtttta

SEQ ID NO: 669 - DNA, 60 bp, Eucalyptus camaldulensis
gttgggggaggatacacgtgaaagtgaaagatgccaaccttcagcttctgtaattactga

SEQ ID NO: 670 - DNA, 60 bp, Eucalyptus camaldulensis
aaagcagtgctcatagttggaaccttcgatcgaaagagagagaatcgtgtgatggaagtg

SEQ ID NO: 671 - DNA, 60 bp, Eucalyptus camaldulensis
tcttgagagcttcttggttatgcatttgtaaattagtactttctagaatctgcaaaggac

SEQ ID NO: 672 - DNA, 60 bp, Eucalyptus camaldulensis
gcaaattgaagtttcgactaccatgaagcaactcacctttgtttttgatttacatcattg

SEQ ID NO: 673 - DNA, 60 bp, Eucalyptus camaldulensis
ggtctggatgaactgtgagtagctgtagaaagttactccgtgatcaagaaaggattgcca

SEQ ID NO: 674 - DNA, 60 bp, Eucalyptus camaldulensis
cctgctgttgcataactccactgaagaaactgtatatagaaaggagcttaatgcagggca

SEQ ID NO: 675 - DNA, 60 bp, Eucalyptus camaldulensis
ggttttatgtcttggttcatggttttggtcaatcaactaaagaaagttgcacaggccgca

SEQ ID NO: 676 - DNA, 60 bp, Eucalyptus camaldulensis
gctacctgcaacacatcagttttctgtatatcaattcagacaataaacgagatctttctt

SEQ ID NO: 677 - DNA, 60 bp, Eucalyptus camaldulensis
gttgtgcttataataatccactcgtcttggatcgaacgaaattccaaacatctaaatcaa

SEQ ID NO: 678 - DNA, 60 bp, Eucalyptus camaldulensis
atatgaacatgggtttcatggctgtactagttaataggcaatttggatctgtcttcggtt

SEQ ID NO: 679 - DNA, 60 bp, Eucalyptus camaldulensis
tgtttcatgcgtaaaagtagactgaccatcaaaaccagttctcttaaaaatggcagcaca

SEQ ID NO: 680 - DNA, 60 bp, Eucalyptus camaldulensis
gtcaatcttgcggttagcttagtgattattgtacctaatcttgggactactcggttttaa

SEQ ID NO: 681 - DNA, 60 bp, Eucalyptus camaldulensis
gctgcaataattgttgttttcacatcttccggcagagcagcaaggctaattgcaaaatac

SEQ ID NO: 682 - DNA, 60 bp, Eucalyptus camaldulensis
atcaacccagcgaggagtctcggcgctgctgttatctacaacaaggataaggcctgggat

SEQ ID NO: 683 - DNA, 60 bp, Eucalyptus camaldulensis
ggaaaagaacgcctgagaactttctctcagatgaagaatggcatcttcattttcgtgaaa

SEQ ID NO: 684 - DNA, 60 bp, Eucalyptus camaldulensis
caggtttcgacttttcattgttttacgagtttgtccgtattctgcaacgggtaaggagta

SEQ ID NO: 685 - DNA, 60 bp, Eucalyptus camaldulensis
tgctttaaagttgctgatttggctgatttggctgattaatgaagcaagttgctgcttggg

SEQ ID NO: 686 - DNA, 60 bp, Eucalyptus camaldulensis
tgatggcgtatggagaggatgattattaggcaattcaatcactgaaactttagatgtctg

SEQ ID NO: 687 - DNA, 60 bp, Eucalyptus camaldulensis
tttgagtgggaagagggcaaaaaacttttactgtcccaagcatacaaaccatcagttgat

SEQ ID NO: 688 - DNA, 60 bp, Eucalyptus camaldulensis
cttaaggggtcaacgctcaatatcttgtcaaagaatacgacagaagtacataattctatc

SEQ ID NO: 689 - DNA, 60 bp, Eucalyptus camaldulensis
gaggacaaagctgaaaaacagtttccttcactttgatgatcctagcttctgcgagtggat

SEQ ID NO: 690 - DNA, 60 bp, Eucalyptus camaldulensis
atatttgtcttgaacacgagtacgcattttgacgcaacttctacatgattttggtagtcc

SEQ ID NO: 691 - DNA, 60 bp, Eucalyptus camaldulensis
cagagtctgccgtcagaataattgggagataaaattcaatatccagaagggaacttttga

SEQ ID NO: 692 - DNA, 60 bp, Eucalyptus camaldulensis
aatttgatttacaagatagctggagcttcccatcttgctggatatgggagtcttgaaagt

SEQ ID NO: 693 - DNA, 60 bp, Eucalyptus camaldulensis
gcgaaacaattttgtctccatggacttcataatttctctatttaatccttgagaaacctc

SEQ ID NO: 694 - DNA, 60 bp, Eucalyptus camaldulensis
gggaaagagagggctgatgagttttgatacccaaggggaatttccttataccccatcata

SEQ ID NO: 695 - DNA, 60 bp, Eucalyptus camaldulensis
tatacagtgtatgagctgttgtaattgatgggcacttcaagcactaaaagaggatggttt

SEQ ID NO: 696 - DNA, 60 bp, Eucalyptus camaldulensis
cttgtatgatgatggggattgtaattttccaccccactgaatgtaaattgtcacatgcat

SEQ ID NO: 697 - DNA, 60 bp, Eucalyptus camaldulensis
gtacttgaggtctggtcaaagaaatgaaatttgggtattgtatagaccatgcaaatcacc

SEQ ID NO: 698 - DNA, 60 bp, Eucalyptus camaldulensis
aatggtctatggatggatattcgtggaccgcctttctaactcagagggaatgtgctatga

SEQ ID NO: 699 - DNA, 60 bp, Eucalyptus camaldulensis
tacgagtcatgacctatgtggctatttgtaatcttgaatgacagtgcgtgagattacctg

SEQ ID NO: 700 - DNA, 60 bp, Eucalyptus camaldulensis
atcatccaccaccatctgagaagctgctgtatggaagcttaaccagcgacagtgattttt

SEQ ID NO: 701 - DNA, 60 bp, Eucalyptus camaldulensis
atctatattaggggcccaaattgggctctgatagaagtgaatgttggcaggaatctttat

SEQ ID NO: 702 - DNA, 60 bp, Eucalyptus camaldulensis
tagccttttaagtatcggtgactaactttccacatgaataattgaagcctgtttttatgc

SEQ ID NO: 703 - DNA, 60 bp, Eucalyptus camaldulensis
tcaagttgccatttgaatttacttttttcgtgtggccatgctttgtgattttaaacgcct

SEQ ID NO: 704 - DNA, 60 bp, Eucalyptus camaldulensis
ttccgccgaaaaatgatcaataattgacagcgtgagaaacgatgaatctaattgaagata

SEQ ID NO: 705 - DNA, 60 bp, Eucalyptus camaldulensis
ttcattctgatgcatacccagttgtccagaaatggttccaaacaaccttgaggcttcctg

SEQ ID NO: 706 - DNA, 60 bp, Eucalyptus camaldulensis
cgagattcatacatcacaactctaaacgtttgccaagcgtacacactcaaacggatccga

SEQ ID NO: 707 - DNA, 60 bp, Eucalyptus camaldulensis
cgggtgctgagattgatgtatttgtaaatacatgctgaaattacagcaaggcattaaata

SEQ ID NO: 708 - DNA, 60 bp, Eucalyptus camaldulensis
aaactgcggtttatacaaccaattaaagcgaggttgatgtaattacactctgtttgcttc

SEQ ID NO: 709 - DNA, 60 bp, Eucalyptus camaldulensis
tgtactctaggactgtggtgatgcaatcaaacattggtgatcacattgatcccacagggt

SEQ ID NO: 710 - DNA, 60 bp, Eucalyptus camaldulensis
ggtgatgtaagatgtctggctggatcaatttccattgtatcgtccgaccccatttgtaca

SEQ ID NO: 711 - DNA, 60 bp, Eucalyptus camaldulensis
tgtggcttcaaagacacctctgcgtgttctgccatttcctatatgaagtgaactggtgat

SEQ ID NO: 712 - DNA, 60 bp, Eucalyptus camaldulensis
ggccaaagaatgtttccatgctttattcgatactggcatatcaagcgaagcgattcaaga

SEQ ID NO: 713 - DNA, 60 bp, Eucalyptus camaldulensis
tgcggtgtgcctaggcgtgctctattatgcaaatccttactcagaattccctgttacaat

SEQ ID NO: 714 - DNA, 60 bp, Eucalyptus camaldulensis
gggtcaaagttcatctgaatgcactgaagaagtacacatatggaaagcatatcgtggcgc

SEQ ID NO: 715 - DNA, 60 bp, Eucalyptus camaldulensis
tgccatgtaacgataaatccacattgattcattgaggtatctaataatattgtgtcctgg

SEQ ID NO: 716 - DNA, 60 bp, Eucalyptus camaldulensis
tgttgcagttggtggatatatcttctacaaatacaggctcaggtcgtacatggactcaga

SEQ ID NO: 717 - DNA, 60 bp, Eucalyptus camaldulensis
aagcacaagagtcttttacgttctctatggaactagctttttagatgcataatctcgatt

SEQ ID NO: 718 - DNA, 60 bp, Eucalyptus camaldulensis
tgtaggctcaggtactggggtttatttttcccctggtatattaaactaaatgccgagaat

SEQ ID NO: 719 - DNA, 60 bp, Eucalyptus camaldulensis
agaagcgggtattatcagtcttccttatgggaagaaacctcaattccaacattctccttt

SEQ ID NO: 720 - DNA, 60 bp, Eucalyptus camaldulensis
tcttttgttgctgaactgagttatgatgatgactgaagattattcactggggaagagaaa

SEQ ID NO: 721 - DNA, 60 bp, Eucalyptus camaldulensis
ttttttcgacacatatgttgttcacattgtgcatgagatgtgatttacccagtcttgtaa

SEQ ID NO: 722 - DNA, 60 bp, Eucalyptus camaldulensis
acctcaccagaaatctaaaaaagatttaacagaaatggatgaagaactcatgtccgcctg

SEQ ID NO: 723 - DNA, 60 bp, Eucalyptus camaldulensis
ctgtgaagaatggtggggaaacggttgacttacgtgagtaatgattaaatgggtggataa

SEQ ID NO: 724 - DNA, 60 bp, Eucalyptus camaldulensis
attcattttcttcccaattaaaacactggcgtttgaccaaagcgaaatggctgttttacc

SEQ ID NO: 725 - DNA, 60 bp, Eucalyptus camaldulensis
aatacatgcagtgctttgtgaattgagatgtgattgtacttatgtttatgagaggaacag

SEQ ID NO: 726 - DNA, 60 bp, Eucalyptus camaldulensis
tcgggaggagatgaggatagtgagactgatctctcgtacatggacgagatgtttatgtta

SEQ ID NO: 727 - DNA, 60 bp, Eucalyptus camaldulensis
tttgttcttctgtgtaaattcgagtgaaggtaactttctgttcaatctctgatgaacatg

SEQ ID NO: 728 - DNA, 60 bp, Eucalyptus camaldulensis
tctagcggaggagggtgatttaagaaattagccactgcgcttttcgttttcatgggatgt

SEQ ID NO: 729 - DNA, 60 bp, Eucalyptus camaldulensis
agaggaactacactgacttcccttccgcagttgagattggatgtgcagagtattttccaa

SEQ ID NO: 730 - DNA, 60 bp, Eucalyptus camaldulensis
gattgctgaggaagtgctgtgtgttcctggattgtgaaattgatcttcttcttcttccca

SEQ ID NO: 731 - DNA, 60 bp, Eucalyptus camaldulensis
tgagaatatgcattttttagggggatcaagggagaatcagagatggcatgtagtggctat

SEQ ID NO: 732 - DNA, 60 bp, Eucalyptus camaldulensis
tggaaaatggagttatgttgctttagtaggtagagattgatgtgctttttgttggacttg

SEQ ID NO: 733 - DNA, 60 bp, Eucalyptus camaldulensis
ctgattagcactcaagtttctgttttttcatcaacatgggagtgcttagtggatgcgatt

SEQ ID NO: 734 - DNA, 60 bp, Eucalyptus camaldulensis
tctgaaggcctaaaagaaaaaggaaatcatcgactcatagtactagctcccagttcgaga

SEQ ID NO: 735 - DNA, 60 bp, Eucalyptus camaldulensis
cctcgtagctcatgtattattctgacaagtaaacttatctgtgtaagagcaatagataca

SEQ ID NO: 736 - DNA, 60 bp, Eucalyptus camaldulensis
atgtgacgccccactacgaggttgtattgatcaatttatggaaacggacgcattcccaaa

SEQ ID NO: 737 - DNA, 60 bp, Eucalyptus camaldulensis
gacggaagttacttcctgctcctggggatccggggacaaacgtgggagaggacaagtata

SEQ ID NO: 738 - DNA, 60 bp, Eucalyptus camaldulensis
gaacgaaatctcctcatattcattatttattaaagatacgcgtctaatgggtctctcttc

SEQ ID NO: 739 - DNA, 60 bp, Eucalyptus camaldulensis
aaagagcaggtagactacaaggcacaacagatcaaatgaaccgttcatcccggccataat

SEQ ID NO: 740 - DNA, 60 bp, Eucalyptus camaldulensis
gtatacaactgcgatagacatatggtctgcaggttgtgtgatggctgaactacttcttgg

SEQ ID NO: 741 - DNA, 60 bp, Eucalyptus camaldulensis
ccggtatgggaatgtgaagtctactatgtatgttggtgggtattatttgaaaattgtatt

SEQ ID NO: 742 - DNA, 60 bp, Eucalyptus camaldulensis
aagtatcaccggtggcaattgccttgatgaaacataacattggaagatcgttctgggttt

SEQ ID NO: 743 - DNA, 60 bp, Eucalyptus camaldulensis
tttggaccatttagcccgagattcggtattaccagtgtcgaatttggattgtgaatgtca

SEQ ID NO: 744 - DNA, 60 bp, Eucalyptus camaldulensis
cgcactattttgctttggatgttgctcaatatacgtcggatgactataaaaagaaaattg

SEQ ID NO: 745 - DNA, 60 bp, Eucalyptus camaldulensis
atgtcagatgctattggcgatgcattggacgatgatgaggctgaagatgaaactgaagag

SEQ ID NO: 746 - DNA, 60 bp, Eucalyptus camaldulensis
ggatgatcatgatgatggctctaaggaacccttggttgtcgcatgtagttttctggtgta

SEQ ID NO: 747 - DNA, 60 bp, Eucalyptus camaldulensis
tttggaaatggtaaggtgcacaagtatggaaagagcacgaccggtgaaagctgggatatt

SEQ ID NO: 748 - DNA, 60 bp, Eucalyptus camaldulensis
atgaatgtgctgtgttgaaggacaagcttcaaaatctattcaggatggagcagcagattg

SEQ ID NO: 749 - DNA, 60 bp, Eucalyptus camaldulensis
aagcaattggtccagcagagtttgcagagcaacatgaggaggatcgttaagagagctaat

SEQ ID NO: 750 - DNA, 60 bp, Eucalyptus camaldulensis
aaggcaagggggtgtggagggtgtggatttatcttattttgttctagttggttcaaaaaa

SEQ ID NO: 751 - DNA, 60 bp, Eucalyptus camaldulensis
atgtgcgtggatctgttactttcatgttcaaagcgattacgtttttgtaggatatgaact

SEQ ID NO: 752 - DNA, 60 bp, Eucalyptus camaldulensis
agctgctagtaacttcacgggatgatgacaagtcttatgtgtcgtcgacagagttgtgat

SEQ ID NO: 753 - DNA, 60 bp, Eucalyptus camaldulensis
agataccagagttcgcccgagagcacgacgaggatttgcagtaccgtccgatgatggact

SEQ ID NO: 754 - DNA, 60 bp, Eucalyptus camaldulensis
aaatattacttatgctcctaggggtggtcttatctgaagttctgaatgtgtgagtgtcaa

SEQ ID NO: 755 - DNA, 60 bp, Eucalyptus camaldulensis
gatccgaatctgagccttttgcttgtaagtatcgtaggaggaaaaagaggcatgctccta

SEQ ID NO: 756 - DNA, 60 bp, Eucalyptus camaldulensis
tcaatcggtggttttcgtgcaatgcctgaaacagctttgaattgtcaacttatttatcct

SEQ ID NO: 757 - DNA, 60 bp, Eucalyptus camaldulensis
catcgatgatgttgttgagggaaacaggaacttgttatatcattacatattcggagagtt

SEQ ID NO: 758 - DNA, 60 bp, Eucalyptus camaldulensis
tcgctcctttcgtgaaagtcctggatgaaaagttcggcattgtgaagggtacaatgacaa

SEQ ID NO: 759 - DNA, 60 bp, Eucalyptus camaldulensis
tcctagtcgaattgcaccatgaaatggcgagcatagtagatccattatcaattttggtga

SEQ ID NO: 760 - DNA, 60 bp, Eucalyptus camaldulensis
gtcttcaggtcacagctacaactggtcctacaattgatacagttggtgtcccaagtgaat

SEQ ID NO: 761 - DNA, 60 bp, Eucalyptus camaldulensis
ttatggatcgattatttgtgatccgtgcccaatcaatgaactttttgcagtccaaattcg

SEQ ID NO: 762 - DNA, 60 bp, Eucalyptus camaldulensis
ttcaggagtgtaaagttagattatgcatcagtaccataataatcttggctttgtaaaaaa

SEQ ID NO: 763 - DNA, 60 bp, Eucalyptus camaldulensis
aagtaccggtgtttttgatgtttaaattaaggttttacaaacaaaaaaaagtaaaaaaaa

SEQ ID NO: 764 - DNA, 60 bp, Eucalyptus camaldulensis
tcaacctgggattacgcggagcccgtctattaatcgaccataagtaaaattccttgatca

SEQ ID NO: 765 - DNA, 60 bp, Eucalyptus camaldulensis
ccaactcagaaagtctgagccctagaatttcaagagtctggccggtctatttaagcattt

SEQ ID NO: 766 - DNA, 60 bp, Eucalyptus camaldulensis
tgaactggagagcaaactcctgttattggaaaattgagaacctaatggtgctgcattgca

SEQ ID NO: 767 - DNA, 60 bp, Eucalyptus camaldulensis
cattgcagtaataaggtattggataataatgaggagggattaaaatggggtgggatctgc

SEQ ID NO: 768 - DNA, 60 bp, Eucalyptus camaldulensis
agggggtgctgttatatacgacaggggcagtgaccatgatcatcaatgtgtcataaagat

SEQ ID NO: 769 - DNA, 60 bp, Eucalyptus camaldulensis
ttgtcgggtcaatcattgacttgtacaagaggcgcccaaatggtgaggcagattattgat

SEQ ID NO: 770 - DNA, 60 bp, Eucalyptus camaldulensis
ttcatctacaacttcacgatcacggggcagagagggacgcttctctggcacgcgcacatt

SEQ ID NO: 771 - DNA, 60 bp, Eucalyptus camaldulensis
agggatgcaagagtctactccagtgagaatagtacctttctcaagccaagtacctgtgat

SEQ ID NO: 772 - DNA, 60 bp, Eucalyptus camaldulensis
gatgaactgatcgtggatgggaatgacatcgagcttgtttcccggtcttgcgccctcata

SEQ ID NO: 773 - DNA, 60 bp, Eucalyptus camaldulensis
acccattgaatgaacaaatgccagtgtgcagttactgaatgaattgatcaattttatagc

SEQ ID NO: 774 - DNA, 60 bp, Eucalyptus camaldulensis
acagactttcctgcttcctctgtactcgtcttcataataaagcgaggagtgccgtcaaaa

SEQ ID NO: 775 - DNA, 60 bp, Eucalyptus camaldulensis
tgttttgatggctgtgccgtgtgaggttgatgaatgatttgaccaaatgcttctctggtt

SEQ ID NO: 776 - DNA, 60 bp, Eucalyptus camaldulensis
tggctatccttcttgtggtgaagaagtggagacccttgatttgaattggcaatggcttgt

SEQ ID NO: 777 - DNA, 60 bp, Eucalyptus camaldulensis
atgttcctgtatgacagactttctgcgacacaaattgcacatttaaggacacccgaatca

SEQ ID NO: 778 - DNA, 60 bp, Eucalyptus camaldulensis
ctttctttccgtaccagctgagagtctactattggtcatttttcaatgcagttcaggcct

SEQ ID NO: 779 - DNA, 60 bp, Eucalyptus camaldulensis
attagattggtttgaaatgacatgccggagcagaaaacatcactttgtgtgctgaaaaaa

SEQ ID NO: 780 - DNA, 60 bp, Eucalyptus camaldulensis
ttacaggactcaagatttgtgaatcctttttctttcgtcggttgttcagggaatgaaaag

SEQ ID NO: 781 - DNA, 60 bp, Eucalyptus camaldulensis
attttacacctttgacttttcacatgatatgaaagtttgtattacctgttttgagttgga

SEQ ID NO: 782 - DNA, 60 bp, Eucalyptus camaldulensis
atggccattgtgaatctaggaatgctttatgctgtcgtgctctttttatgttgcagacat

SEQ ID NO: 783 - DNA, 60 bp, Eucalyptus camaldulensis
tttgtcggttcaatatgtttatgattggttagcaactctgagaaataaactctcttggtg

SEQ ID NO: 784 - DNA, 60 bp, Eucalyptus camaldulensis
gtccagtgctattgtcatattcgctatgctgtcgagtgtggttacggtaatctagcgatt

SEQ ID NO: 785 - DNA, 60 bp, Eucalyptus camaldulensis
ctcctctagaaagggaattacccaaaagttgattggtttcggaaaagggaaaatagaggg

SEQ ID NO: 786 - DNA, 60 bp, Eucalyptus camaldulensis
gagctatcccatggacatgacgccatggtgtccctccaggggagattagccctaccccga

SEQ ID NO: 787 - DNA, 60 bp, Eucalyptus camaldulensis
aaagccagagaatcaggaggggcatcacgtgagtcaaggggatgattgcttcaatttttt

SEQ ID NO: 788 - DNA, 60 bp, Eucalyptus camaldulensis
acgtgcaacaaccatgtataaaccacaaaataaaatgagtccttcctctttgtgctcgca

SEQ ID NO: 789 - DNA, 60 bp, Eucalyptus camaldulensis
gtacaaggatgagaagttcagcactagagttaaggatgaagacgggtttccctgcttttc

SEQ ID NO: 790 - DNA, 60 bp, Eucalyptus camaldulensis
cgataccgtgtgcttagaattggcatgaacatgtatgttcgcgtacaccatgtcttttct

SEQ ID NO: 791 - DNA, 60 bp, Eucalyptus camaldulensis
ttttggactcgtggtcagcaactcggcaaagggaaaccacgtctttatcggattgaaatt

SEQ ID NO: 792 - DNA, 60 bp, Eucalyptus camaldulensis
tcgtgttatggtatgttgcctgtataattttacatgaccacccagtgagcggtacctggt

SEQ ID NO: 793 - DNA, 60 bp, Eucalyptus camaldulensis
ttcccttcccccgccccccttgcttgtacattaggtataggttttatgagatccgtctca

SEQ ID NO: 794 - DNA, 60 bp, Eucalyptus camaldulensis
gatcaatacttgcactatacttatgtttgtctgtgctgctttgattacatttggaggtat

SEQ ID NO: 795 - DNA, 60 bp, Eucalyptus camaldulensis
atccctttttatggtgtcctctgggcttgcggaggctttgcaatgaaaaaacaaaaaaaa

SEQ ID NO: 796 - DNA, 60 bp, Eucalyptus camaldulensis
gccagtgttgcattttttcctacctgtaatgacattttgttacatttataagagagagtg

SEQ ID NO: 797 - DNA, 60 bp, Eucalyptus camaldulensis
agagaggtggatattgtatagtgctcagtttgcaatataatgcaggaattctatcgtttt

SEQ ID NO: 798 - DNA, 60 bp, Eucalyptus camaldulensis
tgttagtgatctgatgttgttgctgtgtatgatatggagattggcctcacattctacatt

SEQ ID NO: 799 - DNA, 60 bp, Eucalyptus camaldulensis
cctttgcttcttggtccaagcatcagccttaatctggtatcaccaatctgtgtctactaa

SEQ ID NO: 800 - DNA, 60 bp, Eucalyptus camaldulensis
tgcccaaacatttcgggtacttgaggttttatgatatggtatgatatgatatgatcgtga

SEQ ID NO: 801 - DNA, 60 bp, Eucalyptus camaldulensis
cctgtggataggatgtttgtgcatttcgacttcaccagacatgttgataaggaaaaaaga

SEQ ID NO: 802 - DNA, 60 bp, Eucalyptus camaldulensis
tatatgcgcgttatatccttttccagtgtaagttagcctgtacagctgttgagtatccaa

SEQ ID NO: 803 - DNA, 60 bp, Eucalyptus camaldulensis
gccaatcctttgtgagatttctattttggtttagtctttaaatgatgtcaaacgtgatgc

SEQ ID NO: 804 - DNA, 60 bp, Eucalyptus camaldulensis
gttcaataacaggatcgtgagatccaccaaagaagttggtactttatgcagaccaagtta

SEQ ID NO: 805 - DNA, 60 bp, Eucalyptus camaldulensis
tcctgtactctattttatcagcaaagcaaatggttgacggactgcatttgcatgtccttt

SEQ ID NO: 806 - DNA, 60 bp, Eucalyptus camaldulensis
tggatttagatggcattggcatttggtgcctgtgatcatgtggaagaaacacgacatgca

SEQ ID NO: 807 - DNA, 60 bp, Eucalyptus camaldulensis
actaatacaatcacaagaaaccaagatttctgtcgcgctatggatttaataatcggggtc

SEQ ID NO: 808 - DNA, 60 bp, Eucalyptus camaldulensis
tgcaacagcaacctgttgtaatattttatctcactcgcttttgagagtgatgtatgtatg

SEQ ID NO: 809 - DNA, 60 bp, Eucalyptus camaldulensis
gtggcacaggtggtgtcaactcaattgcagtcctgggcagttggcgtcttctgttttcct

SEQ ID NO: 810 - DNA, 60 bp, Eucalyptus camaldulensis
tcttttttcgaggcaattctattaatttgttttgtggccgagaagcaattgatatttttt

SEQ ID NO: 811 - DNA, 60 bp, Eucalyptus camaldulensis
gccaactccatctttttctgatgtcatcggtattgggaagcttctagaaacggcacttga

SEQ ID NO: 812 - DNA, 60 bp, Eucalyptus camaldulensis
attggacttgggcactgcactatattagtatatcacagagaacattaatgaaggttgtgt

SEQ ID NO: 813 - DNA, 60 bp, Eucalyptus camaldulensis
tgattagcataggttttgcttgaagagtgtggaactgaaatgtgcaaagtcctggttttg

SEQ ID NO: 814 - DNA, 60 bp, Eucalyptus camaldulensis
agtcacgcggtctcattaaccctcttcgaatctttcagtgtatgcttttattcaacaaag

SEQ ID NO: 815 - DNA, 60 bp, Eucalyptus camaldulensis
cagatcaaagagcagtaccacaaacggaacagtgttcctctagatcgggctattgcaaat

SEQ ID NO: 816 - DNA, 60 bp, Eucalyptus camaldulensis
tctgctcgaaactgctttaacaaactgcaggctcaatatattggagtgttgtgatttaga

SEQ ID NO: 817 - DNA, 60 bp, Eucalyptus camaldulensis
gtatgaaattcggcttgcaattgtacaaaaaggaaggtatataaaaggatcattcgcttg

SEQ ID NO: 818 - DNA, 60 bp, Eucalyptus camaldulensis
agtcgcagctagaggctctccttttcttttcttttttccgtcagtagaggtgatcctgtt

SEQ ID NO: 819 - DNA, 60 bp, Eucalyptus camaldulensis
ggatcaaagtcagggaaaaagcgacaaagaggtgcttaatccttagcagctaccaaagca

SEQ ID NO: 820 - DNA, 60 bp, Eucalyptus camaldulensis
tagatgaacgtacgaaaacaggaggcatagctggacctaataatatgcatgttcttcgca

SEQ ID NO: 821 - DNA, 60 bp, Eucalyptus camaldulensis
tgtgagatacgatgttgcgcccatcgaaaagaagggtgctgcaatcaataagacgtagat

SEQ ID NO: 822 - DNA, 60 bp, Eucalyptus camaldulensis
ggtctgttgatgaagggatgatgatttgtttctggttttcttttcttttgaactgattca

SEQ ID NO: 823 - DNA, 60 bp, Eucalyptus camaldulensis
gtggctgctttgctttgtgttaagtaatgtgtttatcatgacttttgaactagtgatttc

SEQ ID NO: 824 - DNA, 60 bp, Eucalyptus camaldulensis
aggtgtgtgcatggcttgaatgtgtaattagacctttggagaaaccattctctgtacaac

SEQ ID NO: 825 - DNA, 60 bp, Eucalyptus camaldulensis
tatcatgaaataggtcaggctggcaatcccagtagagaatgatataaagaaattttgact

SEQ ID NO: 826 - DNA, 60 bp, Eucalyptus camaldulensis
ctaccttctgtactcgtaataacgtggcgcaataccaaaatttgcagtcatcagaatttg

SEQ ID NO: 827 - DNA, 60 bp, Eucalyptus camaldulensis
cccttccctgtgttcactgctttcagttttcatgtacttatttgttgtgttattgctgag

SEQ ID NO: 828 - DNA, 60 bp, Eucalyptus camaldulensis
tttgctgttgttacccttgtgcagttcctgatgaattgttaagtttttcagcattctaaa

SEQ ID NO: 829 - DNA, 60 bp, Eucalyptus camaldulensis
agagtgggatttgaacccacgcccttccggaccagaaccttaatctggcgccttagatca

SEQ ID NO: 830 - DNA, 60 bp, Eucalyptus camaldulensis
gctccgtctagtcaatcctttttcgcattgattgtgaggaccagtgtcttggttctcttt

SEQ ID NO: 831 - DNA, 60 bp, Eucalyptus camaldulensis
tacgtcttgaactgtgtatacttcgatgttttaccaggaaattataataaaaacggaggc

SEQ ID NO: 832 - DNA, 60 bp, Eucalyptus camaldulensis
aaattgtcttgcatacgtgaactccggttaccatcagatacagtttcaaggttaaactcg

SEQ ID NO: 833 - DNA, 60 bp, Eucalyptus camaldulensis
gttcagctcttctttctgcgttaaaatttgtgaagtggtgtgttttttggctttctgaat

SEQ ID NO: 834 - DNA, 60 bp, Eucalyptus camaldulensis
tttgctgaggggtgcattggattgtaatgcatgagcaacaggaatttgccaaaagcgaga

SEQ ID NO: 835 - DNA, 60 bp, Eucalyptus camaldulensis
gtttggtggtgtggaaatgtatttggatcgaagctgagggtttaatactaataaaaaatg

SEQ ID NO: 836 - DNA, 60 bp, Eucalyptus camaldulensis
caatcaaacggagacataaagcacgtcctggggttaaatgagtcggcacaatcagtaaga

SEQ ID NO: 837 - DNA, 60 bp, Eucalyptus camaldulensis
gcatgggcatctgctcataccctgagttttaagctcctttgctacctttcagggaatttt

SEQ ID NO: 838 - DNA, 60 bp, Eucalyptus camaldulensis
ttgtctggggacttcattagtatacagactgttgatgtcctatgaacaagcacagaatgt

SEQ ID NO: 839 - DNA, 60 bp, Eucalyptus camaldulensis
atatgtgtttaaccatgacaacatggcaaggaggtctctgaagaacaccgcccttgcata

SEQ ID NO: 840 - DNA, 60 bp, Eucalyptus camaldulensis
tccagatcagcggcgagcggaacatggagaaggaggacaagaacgacacctggcaccgga

SEQ ID NO: 841 - DNA, 60 bp, Eucalyptus camaldulensis
tcttcagtgtatgtatcctttgtacacttctgaaattcgtaggccgccctatgtgatcat

SEQ ID NO: 842 - DNA, 60 bp, Eucalyptus camaldulensis
atggaaagtgccgctagtaatgaataaaggcagaccatgaaggtttggttacacgatgtg

SEQ ID NO: 843 - DNA, 60 bp, Eucalyptus camaldulensis
aactccggttgatgagatgatgagacatgcatatgtggatcccaatgaccctaccaagat

SEQ ID NO: 844 - DNA, 60 bp, Eucalyptus camaldulensis
ttcgcattctcgtcgcgaaattccgatccgatcgagagatcgcagagagagagagagaga

SEQ ID NO: 845 - DNA, 60 bp, Eucalyptus camaldulensis
ttttttcacccactcattcatttaatttaaatccctgtaacaccataggcacgagtagag

SEQ ID NO: 846 - DNA, 60 bp, Eucalyptus camaldulensis
ctctctctctctctccatttccttaagactcaatttcgagtgtaaatgaattttatcaag

SEQ ID NO: 847 - DNA, 60 bp, Eucalyptus camaldulensis
taaacagacccaatcaaatctacagtagcgagagcagtttcgactggagcttcggaaagt

SEQ ID NO: 848 - DNA, 60 bp, Eucalyptus camaldulensis
ggtgctggtttgttagattcttacctggtgatttgtgctttgactcttgtgcttcaatga

SEQ ID NO: 849 - DNA, 60 bp, Eucalyptus camaldulensis
tggccacagctgcttcaagtttagcgatgaagccagaattagagcgaagagaaagaagtt

SEQ ID NO: 850 - DNA, 60 bp, Eucalyptus camaldulensis
gtttggttcatgcactgtcacttcgacgtccatttgagctggggcttgaagatggcgtgg

SEQ ID NO: 851 - DNA, 60 bp, Eucalyptus camaldulensis
catcttaggcatggtgtttgaaggacaaagatcggttggtttatctcaaaatcgcctcca

SEQ ID NO: 852 - DNA, 60 bp, Eucalyptus camaldulensis
tgtggtgttctgcctatgcagacaaagttctacattgaaagctgtggaagaacatgaaga

SEQ ID NO: 853 - DNA, 60 bp, Eucalyptus camaldulensis
cgtgtgattgacataatctattagactaagctgttgcgcatcttcttggtaaacaaaaca

SEQ ID NO: 854 - DNA, 60 bp, Eucalyptus camaldulensis
gcacattctttgtcaaaaggtttcgttttgtaatcaatcgagcaataacaattccgcggc

SEQ ID NO: 855 - DNA, 60 bp, Eucalyptus camaldulensis
tgttgagaataaagggcctggacattgaaacgatccagcaagcttacactgtctgaagga

SEQ ID NO: 856 - DNA, 60 bp, Eucalyptus camaldulensis
gtcctgataacaaggctgtttatcattcgtttgagtggaacctatgtacagagagagttt

SEQ ID NO: 857 - DNA, 60 bp, Eucalyptus camaldulensis
gacgggtatgtggctagagagaaggaagacccccacaaactgttctatcttgtattactt

SEQ ID NO: 858 - DNA, 60 bp, Eucalyptus camaldulensis
gtctgatatggcaatgttaaataactacagtgactctctacttgtaatgagatctgagat

SEQ ID NO: 859 - DNA, 60 bp, Eucalyptus camaldulensis
tccaatttcgttttcccttgtccaatgtgcaattttgtggttagcgtgttgcattgttca

SEQ ID NO: 860 - DNA, 60 bp, Eucalyptus camaldulensis
ggggaattgtgcgtcataggctgttgatgtgtggcatatatagcatttccagggaacaat

SEQ ID NO: 861 - DNA, 60 bp, Eucalyptus camaldulensis
tgtcgaacttcccctcagtacttgtatatgaatttgtgcgaacagcttgtgagatttaaa

SEQ ID NO: 862 - DNA, 60 bp, Eucalyptus camaldulensis
tcggttctttgacttatgggttgatgttgtaaatggttgttggatgcaagcgacatcgca

SEQ ID NO: 863 - DNA, 60 bp, Eucalyptus camaldulensis
agcatctgcatctgatatcctttagggactcctagcccactcattgtttttctagtatca

SEQ ID NO: 864 - DNA, 60 bp, Eucalyptus camaldulensis
cgccaacttcaatcagaacatcgattacgtcttcaagatcgtcttgatcggcgactctgc

SEQ ID NO: 865 - DNA, 60 bp, Eucalyptus camaldulensis
tgggattgaactctgtcgtcgttcttgttccgacgaaagcagaaatcaagaacagcagat

SEQ ID NO: 866 - DNA, 60 bp, Eucalyptus camaldulensis
cgatggatcgtattgaagatcatccgaagcttaacagttcgctcatgacttgcataaaac

SEQ ID NO: 867 - DNA, 60 bp, Eucalyptus camaldulensis
cgagagactcgtctttgcatattacaaggcaaaatataatgctgtatcaccatggggaat

SEQ ID NO: 868 - DNA, 60 bp, Eucalyptus camaldulensis
ccgtcttccggagaccctttctcttttccagctgataactggcactcggaccgaaagctc

SEQ ID NO: 869 - DNA, 60 bp, Eucalyptus camaldulensis
tccattgcagctgctgagaatggatcatcaagcactactgcaagtgaacctgtttttgct

SEQ ID NO: 870 - DNA, 60 bp, Eucalyptus camaldulensis
tatcgtttatttttggggtgccgtggatcactgcttaatacgaaaatgtagtgaaatgtg

SEQ ID NO: 871 - DNA, 60 bp, Eucalyptus camaldulensis
gagaccacactgtctctgctactcatggtgacatggaccagaatactcgtgatataatca

SEQ ID NO: 872 - DNA, 60 bp, Eucalyptus camaldulensis
agagctaaatacccatcacttgacatagaggttgatggcggtttgggaccttctaccatt

SEQ ID NO: 873 - DNA, 60 bp, Eucalyptus camaldulensis
cccatgtttctattctatgtaagcttttctaggagagatgattattagtagatgggtttg

SEQ ID NO: 874 - DNA, 60 bp, Eucalyptus camaldulensis
ctctgataagattggagaaatcccaagtggatcatactaggctataagcgttttcttccg

SEQ ID NO: 875 - DNA, 60 bp, Eucalyptus camaldulensis
atccaaatgctttttgcagtttggtcatggacgacgcagttgttccagatctgtgagatt

SEQ ID NO: 876 - DNA, 60 bp, Eucalyptus camaldulensis
ctgatgtggatgaagctcgagctcttccacggtcctactttatatggtgggtgtataatt

SEQ ID NO: 877 - DNA, 60 bp, Eucalyptus camaldulensis
ttggttatctcctgtcaggagcttattggcctgttccttggtaccgcttgtttgaaagat

SEQ ID NO: 878 - DNA, 60 bp, Eucalyptus camaldulensis
attggcttgggtcgtaagtgtcaaagctgcgaagaaatgcatactcggtctccattaaca

SEQ ID NO: 879 - DNA, 60 bp, Eucalyptus camaldulensis
ggctcaccaacaacaatttagcctgaagtttaatagatcggcctttttctttgcggcagc

SEQ ID NO: 880 - DNA, 60 bp, Eucalyptus camaldulensis
taggatcagctttcctgtcacagcgggacgtttctgtgcttgaattggttgagtattgta

SEQ ID NO: 881 - DNA, 60 bp, Eucalyptus camaldulensis
tgcttcttgtattgattgacagtgattgtgggtttacaattattcattgcttgcttgaac

SEQ ID NO: 882 - DNA, 60 bp, Eucalyptus camaldulensis
ggtgatgtgtttggctttagctggaagtatggtttatcaatcgtacaaatgaagagttta

SEQ ID NO: 883 - DNA, 60 bp, Eucalyptus camaldulensis
cagacgagagaaacatctctgtaaattatccaggtttcgggtaagatattctttgtgcct

SEQ ID NO: 884 - DNA, 60 bp, Eucalyptus camaldulensis
gtagagaagaatgatttgctttccttttagaaatctccgggactaatttaatttatgcac

SEQ ID NO: 885 - DNA, 60 bp, Eucalyptus camaldulensis
acgtttatttgttatcggtgattggcacatttgcttcataacttgtattgctggtggtcg

SEQ ID NO: 886 - DNA, 60 bp, Eucalyptus camaldulensis
cgggcgccggggtacgggcatcccccggcgggatacgggttgctcggcgggctgctgatc

SEQ ID NO: 887 - DNA, 60 bp, Eucalyptus camaldulensis
atggtactcttgccatatgcagaatgtactgctcaacttatgcaatgagactcatgtcga

SEQ ID NO: 888 - DNA, 60 bp, Eucalyptus camaldulensis
ttggaccctctaaagtatttgtttgaggtggggtaagaaccatttttgtaacaagccatc

SEQ ID NO: 889 - DNA, 60 bp, Eucalyptus camaldulensis
agtgcaagttttgacagtgctgtaaactgaatcatcagttgaaagtttgggcttctgatt

SEQ ID NO: 890 - DNA, 60 bp, Eucalyptus camaldulensis
tccgggaagatactgaggaaagatctcagggccaagttagctacggcggcccccatttct

SEQ ID NO: 891 - DNA, 60 bp, Eucalyptus camaldulensis
agactataataagctattatattcacagatcttagaacccttgagagctatggttacggg

SEQ ID NO: 892 - DNA, 60 bp, Eucalyptus camaldulensis
ctcccttaagcacattcactttttataagggggtattgaatgcgacttttgcgatgtgtg

SEQ ID NO: 893 - DNA, 60 bp, Eucalyptus camaldulensis
tatggttgccccaagtatcaccaacattacacttggtgttacgccgaagacttgttgcat

SEQ ID NO: 894 - DNA, 60 bp, Eucalyptus camaldulensis
ggatcatctttgtatgtgcgtggctccatcagcagaaattgtcatgtataatgtcactca

SEQ ID NO: 895 - DNA, 60 bp, Eucalyptus camaldulensis
ttgatggccaggtccatttttgatatttgatggctagatctagactttgcattttccttt

SEQ ID NO: 896 - DNA, 60 bp, Eucalyptus camaldulensis
aatcgtcacatgcagtgatgcttgcctagggtacttatactggtctgtggctcaaattgt

SEQ ID NO: 897 - DNA, 60 bp, Eucalyptus camaldulensis
gaactgatttggactgagctttcaggaacacttgagtctacgttgcaatggaaagctatt

SEQ ID NO: 898 - DNA, 60 bp, Eucalyptus camaldulensis
gtgcggatgactaaactttcagatcgaataagggattaaatgagatgactgaactggttt

SEQ ID NO: 899 - DNA, 60 bp, Eucalyptus camaldulensis
gctgcctggctttgcggataaagtgatgcagtaacattcagtgaattgcttttcaacctg

SEQ ID NO: 900 - DNA, 60 bp, Eucalyptus camaldulensis
taaaatatccacacgcgtttcacatgtataagagtatgtatcgagcagaggggcgatggc

SEQ ID NO: 901 - DNA, 60 bp, Eucalyptus camaldulensis
tagggatactgaacatggccgtctaatgaataagattacaggtcttgtgcgttgtccaca

SEQ ID NO: 902 - DNA, 60 bp, Eucalyptus camaldulensis
aattttctctgagtacacgctgtgcttttactattacataaatctggacgaggtagtgct

SEQ ID NO: 903 - DNA, 60 bp, Eucalyptus camaldulensis
acttgatgatacatgtgttgcataccattgcaacctatcaaatgcgttctcctggatgca

SEQ ID NO: 904 - DNA, 60 bp, Eucalyptus camaldulensis
ttgggtgaaagggtcatgcttccggttggggacaatgttttagcctgtaactttgttaca

SEQ ID NO: 905 - DNA, 60 bp, Eucalyptus camaldulensis
gttgccggagttcgtcccaagctttgtattcagcttcacggttgagacaaatagaagttt

SEQ ID NO: 906 - DNA, 60 bp, Eucalyptus camaldulensis
tatgtactggaagttacctggagagagcatgattgagcagccaaataaaccgatcggcaa

SEQ ID NO: 907 - DNA, 60 bp, Eucalyptus camaldulensis
aattgtttgatgtggctttgattaaaattttgggagcctgcttgcttgcttgcttgcttg

SEQ ID NO: 908 - DNA, 60 bp, Eucalyptus camaldulensis
gcccgaaacctttggtcgagggcacgtttgcctgggtgtcacacatggcgttgcaaaaaa

SEQ ID NO: 909 - DNA, 60 bp, Eucalyptus camaldulensis
ccgccgagatgtatcgtgaaatggaaatacttgggcatatgtttgcctatgttttcctaa

SEQ ID NO: 910 - DNA, 60 bp, Eucalyptus camaldulensis
aattttggagctgttgagagtatagttattttattttactggacagttccgttgcttctc

SEQ ID NO: 911 - DNA, 60 bp, Eucalyptus camaldulensis
acatctccttgtatacttggatccttgtgtaaaaactcttatatgtgaattacaccatcc

SEQ ID NO: 912 - DNA, 60 bp, Eucalyptus camaldulensis
gagcaggcggagatggtggcttttgagcagttacttctattttcagcttttacttgctct

SEQ ID NO: 913 - DNA, 60 bp, Eucalyptus camaldulensis
ttttgtttgaagaccttgatatttgctattcggtaattgtttccatgtgctggtctggga

SEQ ID NO: 914 - DNA, 60 bp, Eucalyptus camaldulensis
gaagtgctcacacaaatataccacgtggtgagtcgaaaagcacttgatgttggggaagat

SEQ ID NO: 915 - DNA, 60 bp, Eucalyptus camaldulensis
cattttgcgactttgcgccgtttctcctgtgatactacacattagtatgggaaagctagt

SEQ ID NO: 916 - DNA, 60 bp, Eucalyptus camaldulensis
agttctgaaacttttgacggctgttatggcacccttgtatttgtctgcctctttttggtg

SEQ ID NO: 917 - DNA, 60 bp, Eucalyptus camaldulensis
tgggcactttttattgctttgcatatggaagaacgatacgagttgtctgcaacaattgat

SEQ ID NO: 918 - DNA, 60 bp, Eucalyptus camaldulensis
aaacgtcgagtttgatgtggggtttcgtcagctgttatggtatggacgcatgtgatcata

SEQ ID NO: 919 - DNA, 60 bp, Eucalyptus camaldulensis
ccgagcgccggatcctgcaggccataatggccgattcccggtaccaacacatgcgcgctc

SEQ ID NO: 920 - DNA, 60 bp, Eucalyptus camaldulensis
ctgtctcctacgttagtattttgtgccgattccgtgtgattgctttgcttctttcgtgca

SEQ ID NO: 921 - DNA, 60 bp, Eucalyptus camaldulensis
tgaaaagccctagaagctttccctttttcctttctatgtccaatttctttcaatatgcaa

SEQ ID NO: 922 - DNA, 60 bp, Eucalyptus camaldulensis
tgctgaatatctgattactgtttttcatactctataaagcagtgttgtcgaagtgtttcc

SEQ ID NO: 923 - DNA, 60 bp, Eucalyptus camaldulensis
ttgatgctagcgcggacacattgtattattttctgagggaatcatgacgttgaattggaa

SEQ ID NO: 924 - DNA, 60 bp, Eucalyptus camaldulensis
tagtgagtgataaagctgggtgaacttttctgtcctttgtctctctgtaattttatgcat

SEQ ID NO: 925 - DNA, 60 bp, Eucalyptus camaldulensis
tcagtggaaataatccccaattcttcatcttgcttctactcgaatcacttcaccggaatt

SEQ ID NO: 926 - DNA, 60 bp, Eucalyptus camaldulensis
ttgtgaattgcttgatgagcattggtaatgcctatgcccatcaaatgggcaggttggatt

SEQ ID NO: 927 - DNA, 60 bp, Eucalyptus camaldulensis
tttgcaagaaaagaaaaccacctttatcccgattgatttgctttgtccgcgttgtgttga

SEQ ID NO: 928 - DNA, 60 bp, Eucalyptus camaldulensis
acgggggaatttaattagctcttttgctatgaagctgctaatttgggatctttcgttaat

SEQ ID NO: 929 - DNA, 60 bp, Eucalyptus camaldulensis
acctacattctctaccagatcctgtagctgattgtgctgtttatattcatatagttcact

SEQ ID NO: 930 - DNA, 60 bp, Eucalyptus camaldulensis
ttgtctcatctctctgtctgatgcacagggaggtcatccctgcaaatgaagtgaaaagtt

SEQ ID NO: 931 - DNA, 60 bp, Eucalyptus camaldulensis
gtgaactcctcttttgcataatatatgttggtcatcaaatctttttgatgggtccttgtt

SEQ ID NO: 932 - DNA, 60 bp, Eucalyptus camaldulensis
aatccctttgcgagtttatttgtagtaagatatgtttcattcgtatgttttggctatcgg

SEQ ID NO: 933 - DNA, 60 bp, Eucalyptus camaldulensis
gcactagcagtttctattttggttattgctttcttcaatatccaaatgatgatgtacagc

SEQ ID NO: 934 - DNA, 60 bp, Eucalyptus camaldulensis
cgaacaccaatttcttctaggttgcaaaattacttttctctatgaatttgctttttccgg

SEQ ID NO: 935 - DNA, 60 bp, Eucalyptus camaldulensis
aatatgcagcttagtttccatatgcttattgcagagcacaatgaactttctaattgagtc

SEQ ID NO: 936 - DNA, 60 bp, Eucalyptus camaldulensis
actggtggtgggttgctgtctgttttttacctcacataaattatgatgttaagctataat

SEQ ID NO: 937 - DNA, 60 bp, Eucalyptus camaldulensis
gatgtactatgttgccatccagtcaaggattggcgtctccacatggtctcaagtaatgaa

SEQ ID NO: 938 - DNA, 60 bp, Eucalyptus camaldulensis
gagctgcattatgattttgtacagacagtttttaagtagagcagaacttcgcctctcttg

SEQ ID NO: 939 - DNA, 60 bp, Eucalyptus camaldulensis
tttaagtgataatcagtttcattaagcaagcaatgagaggtccggacaattttgttgtgc

SEQ ID NO: 940 - DNA, 60 bp, Eucalyptus camaldulensis
ccattatgccgggtattatatgtgccgacatacgagttttcgtgtgctaataagtaaaaa

SEQ ID NO: 941 - DNA, 60 bp, Eucalyptus camaldulensis
accccccagggatcaactttgtaattgcatttgtgaacggaatcgtcttgctattttctt

SEQ ID NO: 942 - DNA, 60 bp, Eucalyptus camaldulensis
tgtccgacttgtaggtcgtaggacaattcatttttaagagccggtcttttgtagactttt

SEQ ID NO: 943 - DNA, 60 bp, Eucalyptus camaldulensis
tgctgtaattaggctgcatttttagtgattagttgtaatagtgggtacagaccaagggtt

SEQ ID NO: 944 - DNA, 60 bp, Eucalyptus camaldulensis
ggacaggatgagtttgcctggaagcttatctattgtcagcgactgtgtagaagctgaatt

SEQ ID NO: 945 - DNA, 60 bp, Eucalyptus camaldulensis
atggcacacaaagaagggaagcttattgatgtgcagagcttgaaatcatttctaggagaa

SEQ ID NO: 946 - DNA, 60 bp, Eucalyptus camaldulensis
tcagccgcgcagcaagtgtaacagattttgaaattaaaggagaaataatattttagcatc

SEQ ID NO: 947 - DNA, 60 bp, Eucalyptus camaldulensis
cgactctgaactctgtgtttcctgtaatttggcgttgcttttcgggaataaaagagtggc

SEQ ID NO: 948 - DNA, 60 bp, Eucalyptus camaldulensis
tgggttacctaatgagatattttcagacgcgggattaaatttttccctcagattcttctg

SEQ ID NO: 949 - DNA, 60 bp, Eucalyptus camaldulensis
tggtttacgagactggtacatgttgtcgctgctggttatttgtatgattgaacaagtgaa

SEQ ID NO: 950 - DNA, 60 bp, Eucalyptus camaldulensis
ggtttgaccttgccagtggaagtgtaaacctcagattctctttttgaagtgtcgtaatgg

SEQ ID NO: 951 - DNA, 60 bp, Eucalyptus camaldulensis
ttctcatgatctgtaattttcttcagctgaatacctgtgccatcgtattgggagttgatt

SEQ ID NO: 952 - DNA, 60 bp, Eucalyptus camaldulensis
tttagggtgtttttaatgtgcaaaatcccaagagatgggcccgtcacatgtaaatttggt

SEQ ID NO: 953 - DNA, 60 bp, Eucalyptus camaldulensis
tttgctgatcaaagagacaattatgagcagacagagcccgctacttacaacaatgctccg

SEQ ID NO: 954 - DNA, 60 bp, Eucalyptus camaldulensis
attatcatctttgcgctcccgcacttcttattgaatcgacagagttcgggtctcgagtta

SEQ ID NO: 955 - DNA, 60 bp, Eucalyptus camaldulensis
ggaattggtgcaaaagagtcgcaggtttcttggttccgttcttccgaaagctttccttat

SEQ ID NO: 956 - DNA, 60 bp, Eucalyptus camaldulensis
agaaggagttggagtactgtgactcatgttatacggaccaaattagtgcgtgaatgggtt

SEQ ID NO: 957 - DNA, 60 bp, Eucalyptus camaldulensis
gcgagccatgtatggcaattatttactgattcaaattttgtgaatccccctggttttgac

SEQ ID NO: 958 - DNA, 60 bp, Eucalyptus camaldulensis
acgcgtgaattactccagtgttagaccatagattatacgtgaatccactaattttcttct

SEQ ID NO: 959 - DNA, 60 bp, Eucalyptus camaldulensis
cattgtgtatcagttcagatactctcagttacttgtctgctgttgaccaccagaacaggg

SEQ ID NO: 960 - DNA, 60 bp, Eucalyptus camaldulensis
aaacgtggttgtggcagtgaacatgttttcaacagatactgaagcagagttgaatgcggt

SEQ ID NO: 961 - DNA, 60 bp, Eucalyptus camaldulensis
gttctgatggttctatactttgaataaatttgaggctatttagtctctagcgtcattacc

SEQ ID NO: 962 - DNA, 60 bp, Eucalyptus camaldulensis
aattcatggctgttgacagatttacctactatatatgggaaggcacgaacgaactgattt

SEQ ID NO: 963 - DNA, 60 bp, Eucalyptus camaldulensis
gtgttttgcagaaacgagtaaaacccgtatttaattttcacaacccccattccaaccctc

SEQ ID NO: 964 - DNA, 60 bp, Eucalyptus camaldulensis
gctaatatcgagtaacaacatgctggtcatgacggtgaacttcacgggtgcactttttca

SEQ ID NO: 965 - DNA, 60 bp, Eucalyptus camaldulensis
tggaacatggtcagggtatttgtataatttggtttcgaggtgactattctcggccactgg

SEQ ID NO: 966 - DNA, 60 bp, Eucalyptus camaldulensis
tttagatggggtccatctattcggtgaaggacatgggatcctactgtttgcaactgttct

SEQ ID NO: 967 - DNA, 60 bp, Eucalyptus camaldulensis
aagatgatttcctgcccttctgtgcactccatagatcgccctgaggataatatatgcctt

SEQ ID NO: 968 - DNA, 60 bp, Eucalyptus camaldulensis
ttcgaaatttggaaagaatttacccgaagggcccgagtgatcaatgtcaatgctgatctt

SEQ ID NO: 969 - DNA, 60 bp, Eucalyptus camaldulensis
actcgttagttctttggatatgtaattggtgttcagagtaaattgaattcccagattgcc

SEQ ID NO: 970 - DNA, 60 bp, Eucalyptus camaldulensis
gaatacgaagaacactggatgctgggaactgtgaggtatttgaatgattaagctgtgttt

SEQ ID NO: 971 - DNA, 60 bp, Eucalyptus camaldulensis
aaggatgaggtttctgatgcaatatcacttggtaaaggacagctctcatgtaatctattc

SEQ ID NO: 972 - DNA, 60 bp, Eucalyptus camaldulensis
ttcagtctgccgttagctcttgagccattcattagtgatttagtgtgctttcttcttttc

SEQ ID NO: 973 - DNA, 60 bp, Eucalyptus camaldulensis
ttcaggtaccggtgaatttgacgaggtggaagtttatgagaaaataccagccgacctgaa

SEQ ID NO: 974 - DNA, 60 bp, Eucalyptus camaldulensis
tttgacacacatttatgttgtagtccttgtcgctccatgtccaagaggatctcttttcgc

SEQ ID NO: 975 - DNA, 60 bp, Eucalyptus camaldulensis
accagaatgatgactatccttattggaagtcgtcttaaccattctttgacgccactttgg

SEQ ID NO: 976 - DNA, 60 bp, Eucalyptus camaldulensis
tgatgtctagtgattccgaatgatgtggtgtacacggagtttgctttcttagtgcaagca

SEQ ID NO: 977 - DNA, 60 bp, Eucalyptus camaldulensis
gatacatcctttctgagatgtctgcttttcgacctaagttgggattctgccaaaaaagaa

SEQ ID NO: 978 - DNA, 60 bp, Eucalyptus camaldulensis
gctgtgtactcttctctggattttgataataagatgttgtcttgactgatttaaagtcgc

SEQ ID NO: 979 - DNA, 60 bp, Eucalyptus camaldulensis
gtaacattcgaatgattgcttaaattggaacttaagaacgagttcatagcattttcccag

SEQ ID NO: 980 - DNA, 60 bp, Eucalyptus camaldulensis
gtttgatggactccactgtggctacttctgttggtctgaatttccagctgttttggcttt

SEQ ID NO: 981 - DNA, 60 bp, Eucalyptus camaldulensis
accccttccgggttcagtatctttattagctgtatcaaatcaagactgtctctaactttg

SEQ ID NO: 982 - DNA, 60 bp, Eucalyptus camaldulensis
ggcatctcggttcgagtgttcgattaggaaacttcgctttactttccatggttcttgaat

SEQ ID NO: 983 - DNA, 60 bp, Eucalyptus camaldulensis
attagagaagcctgattatcatctattcctgcctctgattccacggttgtagcgtttgct

SEQ ID NO: 984 - DNA, 60 bp, Eucalyptus camaldulensis
tcgtctcatgaggcatatggctttggacatgatgtaatagtcttgtctgagctctggctt

SEQ ID NO: 985 - DNA, 60 bp, Eucalyptus camaldulensis
tttcgtttctgggtttgaatttgtttgtttcgaaatttgaaaagaatttacccgaagggg

SEQ ID NO: 986 - DNA, 60 bp, Eucalyptus camaldulensis
tagtaatcgttgacacggtgtaagctgatatttttagttggggttgtgctctctctctct

SEQ ID NO: 987 - DNA, 60 bp, Eucalyptus camaldulensis
cacgacttgttaatctgccccagttctgcttaattttgctttgcctggttctactgctgt

SEQ ID NO: 988 - DNA, 60 bp, Eucalyptus camaldulensis
cactgccttccagtgtcttacttcgatagtattccgactcattagatttctgcttaaact

SEQ ID NO: 989 - DNA, 60 bp, Eucalyptus camaldulensis
tggggaataaatagcagaaatcgtttgctttagcgtggacaacatggctgttttgctatc

SEQ ID NO: 990 - DNA, 60 bp, Eucalyptus camaldulensis
tgcgtatactgctcattgttgcaatgcgggatatgtccgattgccttcttgcttcaaagt

SEQ ID NO: 991 - DNA, 60 bp, Eucalyptus camaldulensis
gccatatcttagtttacagagcttgcaaattttgttgttcttagatggaagaccttgcac

SEQ ID NO: 992 - DNA, 60 bp, Eucalyptus camaldulensis
ttagttaaacatggtaggttttgtccttaagttgtttttccttgtttgaacggtcctctg

SEQ ID NO: 993 - DNA, 60 bp, Eucalyptus camaldulensis
tggattgatttctcgaactgtaagtctgtaagggaagtcgagaacaagcacatttgcata

SEQ ID NO: 994 - DNA, 60 bp, Eucalyptus camaldulensis
tggagatgtcatcatctattgcggatgaattcaataatgtaatttctggcgactttcggg

SEQ ID NO: 995 - DNA, 60 bp, Eucalyptus camaldulensis
gccaatccaacagtaccggttgctatttttcttgtattaaagttgtgtggagtttcttat

SEQ ID NO: 996 - DNA, 60 bp, Eucalyptus camaldulensis
ggtggtttctgagttttcatgcaagcaaatgggtttttaataaagtctgagtttttcttg

SEQ ID NO: 997 - DNA, 60 bp, Eucalyptus camaldulensis
tcctcaaaaaagtcattattcttatgttggcctttcaaattgtcatattcctaattcgct

SEQ ID NO: 998 - DNA, 60 bp, Eucalyptus camaldulensis
cgactttgtatatgaggtcatgtgttggattgcgctgtcgaattgcgtgcatttcgcctt

SEQ ID NO: 999 - DNA, 60 bp, Eucalyptus camaldulensis
tggggcttatggtatttataattctactggtgtggcccgagtgtggtttgaatggaggtt

SEQ ID NO: 1000 - DNA, 60 bp, Eucalyptus camaldulensis
acaacttttttgtaagagcaggcagctcatagttgtacatgcctgaattatcaactgatg

SEQ ID NO: 1001 - DNA, 60 bp, Eucalyptus camaldulensis
caagttaaggagctttaacgaataaattctccgcctccaatgtggatagtttttatgaaa

SEQ ID NO: 1002 - DNA, 60 bp, Eucalyptus camaldulensis
cattcggggcctcattgatcccaacaagtactttgtaaaggtcttgagcaacggaatcaa

SEQ ID NO: 1003 - DNA, 60 bp, Eucalyptus camaldulensis
aatggaatgccctgagacaacggggatctggcaataaaatttcccgcacttcattggttt

SEQ ID NO: 1004 - DNA, 60 bp, Eucalyptus camaldulensis
ctatggtgtcttatgattgtctctgtattgatctgaattgattggatgtaattgcgaaag

SEQ ID NO: 1005 - DNA, 60 bp, Eucalyptus camaldulensis
tcaacagccaaacttgcactatgaatttggatacttctgtgctttgtttaatttcatctg

SEQ ID NO: 1006 - DNA, 60 bp, Eucalyptus camaldulensis
tgggatccatgcggcagaatagtcctacagtaaactgttacaccttaacttaggtttaga

SEQ ID NO: 1007 - DNA, 60 bp, Eucalyptus camaldulensis
gatggtgtaataataaacctctgaggtatgatatttatgacggagtacctgaatttttcg

SEQ ID NO: 1008 - DNA, 60 bp, Eucalyptus camaldulensis
gatatccctcggccattcatggttgatctttctcatgtaatgactgccatcctgataatt

SEQ ID NO: 1009 - DNA, 60 bp, Eucalyptus camaldulensis
tgttttttgcttgtaagctctatcttgcaaagtcaatgtgcttcaagtcctttagctgtg

SEQ ID NO: 1010 - DNA, 60 bp, Eucalyptus camaldulensis
cttcgtgctcatacttgcagaaaccaatttgatgtttcaaatgcttgcgcagtcgactct

SEQ ID NO: 1011 - DNA, 60 bp, Eucalyptus camaldulensis
ctgcttctattcatcaatttctggcggtaaatgtcatatgtgatcccgtgatcggtttgg

SEQ ID NO: 1012 - DNA, 60 bp, Eucalyptus camaldulensis
tgaacatggttgcctttgggctgcatcgaaataaatggtgtggggagcatgtgtaacaaa

SEQ ID NO: 1013 - DNA, 60 bp, Eucalyptus camaldulensis
tggaggaatcctccagtgctttaacaaggttctatccaatgtccatagccaccatcgatt

SEQ ID NO: 1014 - DNA, 60 bp, Eucalyptus camaldulensis
acccactgaacataatggaggagctccaacgaaggtacctgacactagtaatggaacttc

SEQ ID NO: 1015 - DNA, 60 bp, Eucalyptus camaldulensis
gttagctgctcctttggattgtgttccgatttgctcgtgattttaatgcttttattcgcc

SEQ ID NO: 1016 - DNA, 60 bp, Eucalyptus camaldulensis
gagttctggcaaagggatgtcgaagaatcaattaggcaggggcatgtgaaaccttttgtt

SEQ ID NO: 1017 - DNA, 60 bp, Eucalyptus camaldulensis
gttcacaggcttttctatagcgaaatgtcgaacttttgtgagactatctccatgacaaca

SEQ ID NO: 1018 - DNA, 60 bp, Eucalyptus camaldulensis
tttggaaacacagatcttctctaggatacggcagaagagggaaaagacctgaacgcgtat

SEQ ID NO: 1019 - DNA, 60 bp, Eucalyptus camaldulensis
ccgggatttgaagactgccaatcttctgatggatgaaaatgaggtggttaaagtggcaga

SEQ ID NO: 1020 - DNA, 60 bp, Eucalyptus camaldulensis
aggagatgttctcgttgttttatgggctatggaagtatctgttcactaagacggagtatc

SEQ ID NO: 1021 - DNA, 60 bp, Eucalyptus camaldulensis
tgaatatactttgtatcaaatcgatatacaagtttttgtcttgagattgccttcaaaaaa

SEQ ID NO: 1022 - DNA, 60 bp, Eucalyptus camaldulensis
taacttctggagctactgtgcattgtatatcaatcaaacccatttttctgatgataactg

SEQ ID NO: 1023 - DNA, 60 bp, Eucalyptus camaldulensis
actgctaatgtgcatatcagcagcttcctagaatagaaccacgtgtggatttagtacgtg

SEQ ID NO: 1024 - DNA, 60 bp, Eucalyptus camaldulensis
ttaagctgtctttgtgcgagagtagtactagtgatgtgtgtgacctccttgtgaagtcct

SEQ ID NO: 1025 - DNA, 60 bp, Eucalyptus camaldulensis
ccaggaaatgcctctcttgtattggatggatgaccatttaaagataggtggcgcttgagt

SEQ ID NO: 1026 - DNA, 60 bp, Eucalyptus camaldulensis
ccagtgttctttccaggtttgcctttggtaaacctatcaactgaatgtcttgtgccacaa

SEQ ID NO: 1027 - DNA, 60 bp, Eucalyptus camaldulensis
agtcagctgggtgtgttcaacgtcgactttgaagcatgatattttgaattttgagctgca

SEQ ID NO: 1028 - DNA, 60 bp, Eucalyptus camaldulensis
ctggagaaagcggctatgaggttcttgttttgtcgtagctgcaaacagcgtaaggaaatt

SEQ ID NO: 1029 - DNA, 60 bp, Eucalyptus camaldulensis
ttttttggatctgtttccagttccatcagacctttctacacagaccatcagtgctttgct

SEQ ID NO: 1030 - DNA, 60 bp, Eucalyptus camaldulensis
ctcacctgaaactagaatactttggtagtgctattttcctgctttgttttgaaagcaggc

SEQ ID NO: 1031 - DNA, 60 bp, Eucalyptus camaldulensis
tgtttcactcattgagcacctgatcgactttttatatcgagatttcagccgatgcatctc

SEQ ID NO: 1032 - DNA, 60 bp, Eucalyptus camaldulensis
tttattaaagtccccaaagttttgaacaaggagtcagagtaacttcatccatctccaggg

SEQ ID NO: 1033 - DNA, 60 bp, Eucalyptus camaldulensis
ctactttgtacctgactactggaatgcaaaatgtcacgctctgggaggttggttgatgtt

SEQ ID NO: 1034 - DNA, 60 bp, Eucalyptus camaldulensis
gatccactgattgtccttaatcatcgcatggcattgatttccgatcgtgctttagctgga

SEQ ID NO: 1035 - DNA, 60 bp, Eucalyptus camaldulensis
tcacatgaagaacatttatccgtgccttcaggtgctcaatctgaatctgcaccttctcaa

SEQ ID NO: 1036 - DNA, 60 bp, Eucalyptus camaldulensis
ggaaacatgtgtggaccagtttgtgttgtaaaatgaacactttcacattgtttcgtttgg

SEQ ID NO: 1037 - DNA, 60 bp, Eucalyptus camaldulensis
tcgcctaaactgtgtcttgaaagattgtttggtgaatggaattttaatccgcgtgtcaaa

SEQ ID NO: 1038 - DNA, 60 bp, Eucalyptus camaldulensis
tttatgaagatttttgtcttttgttttcgggttctgtaaatgcttcaactccgaatgctc

SEQ ID NO: 1039 - DNA, 60 bp, Eucalyptus camaldulensis
ttgttccctacaagaagagacgtcttttgtccacgcatcttctactcttcatgcttagtc

SEQ ID NO: 1040 - DNA, 60 bp, Eucalyptus camaldulensis
attcccttcctgcaagttcttttctaaaagatcaatgattcttgcttttgatgtcgagta

SEQ ID NO: 1041 - DNA, 60 bp, Eucalyptus camaldulensis
gtattctgatcgtcagtctatctgacatcagaaagtggaagcaggctagtccccgaaaat

SEQ ID NO: 1042 - DNA, 60 bp, Eucalyptus camaldulensis
attgtgtcaaccctaggcacgggttcttggtcaattcttagtttgtatgatatctccaat

SEQ ID NO: 1043 - DNA, 60 bp, Eucalyptus camaldulensis
tctttgtcaagacccttactggaaagacaatcactttggaagtggagagctctgatacca

SEQ ID NO: 1044 - DNA, 60 bp, Eucalyptus camaldulensis
ttgtttcccaagactttggagctcctggttaatcgttctgcgtttgttaattgagtttag

SEQ ID NO: 1045 - DNA, 60 bp, Eucalyptus camaldulensis
tgtatcttgatgaagtgagcttggtcatctttcaaaagtgtactgtatctactatgacca

SEQ ID NO: 1046 - DNA, 60 bp, Eucalyptus camaldulensis
cagtctcgacagaatctgatcgacctacagactacctgttctgtttcgatgtgtcctata

SEQ ID NO: 1047 - DNA, 60 bp, Eucalyptus camaldulensis
gatggtagtggaacagagtgaatcgaccgctgaagcaaccaagtagctggtgtgtatctc

SEQ ID NO: 1048 - DNA, 60 bp, Eucalyptus camaldulensis
tgttcgagcaaatgtactgtacttaagctcaaaatactcgaaacccatatcggactctgt

SEQ ID NO: 1049 - DNA, 60 bp, Eucalyptus camaldulensis
attatggtagccaacatgtaatatgactcatatgtgagatgaaactgggaattcaatctc

SEQ ID NO: 1050 - DNA, 60 bp, Eucalyptus camaldulensis
tagcttagcttctgtatttgacttcatgagacgtgacttgcacggagaatagaacacagg

SEQ ID NO: 1051 - DNA, 60 bp, Eucalyptus camaldulensis
agcatgtgtgtgtttgggctttgtcttgtaccggttgtatcttgggtgtttaataatatt

SEQ ID NO: 1052 - DNA, 60 bp, Eucalyptus camaldulensis
gcaatacattgtgcccagcatttagaacattgcatcttctcgaaatttgatttgacatgt

SEQ ID NO: 1053 - DNA, 60 bp, Eucalyptus camaldulensis
tgcatgattagtccacttgacttgtgaaccttgacatttggttcagttttagaaatcctt

SEQ ID NO: 1054 - DNA, 60 bp, Eucalyptus camaldulensis
ttctatagtctggtaccgctgtctggaaccaggcagttcatggtgccttcaacataataa

SEQ ID NO: 1055 - DNA, 60 bp, Eucalyptus camaldulensis
taccagaaggccggcggtgattgagatcgatagagagatagataatttaaattatggagt

SEQ ID NO: 1056 - DNA, 60 bp, Eucalyptus camaldulensis
gttttgatggtttgatgtgactatattctgatattttcttgttccaatcggatgtgctgg

SEQ ID NO: 1057 - DNA, 60 bp, Eucalyptus camaldulensis
agttgtggcttttcatgactccattctggaacgaactatgggtcttaacgatctgtaacg

SEQ ID NO: 1058 - DNA, 60 bp, Eucalyptus camaldulensis
ggtgattttcctagttgtcttctatctcactaggtaatggatcgcaataggttgaaggct

SEQ ID NO: 1059 - DNA, 60 bp, Eucalyptus camaldulensis
acctgacataatgtatctgtgtccaaattttccatccgtttggtgaatacgactcgtcgt

SEQ ID NO: 1060 - DNA, 60 bp, Eucalyptus camaldulensis
tgtgtttggcatgcataatgtacacttctagacatatgaagcacaatgaatgaggtgcat

SEQ ID NO: 1061 - DNA, 60 bp, Eucalyptus camaldulensis
agaggcaactaattcccggatcagtcctgatcatatttatcagaagcggggtggaaacaa

SEQ ID NO: 1062 - DNA, 60 bp, Eucalyptus camaldulensis
gatgcatcttcaataaatttagtgttcctgaatgagagatgttttggtacacgctcgtgg

SEQ ID NO: 1063 - DNA, 60 bp, Eucalyptus camaldulensis
gctagagcattagttctatagatgagagagattttgccattagtttttgttatccgatca

SEQ ID NO: 1064 - DNA, 60 bp, Eucalyptus camaldulensis
ggtctttcttggtcatggtttgatctgttttatggaggcaaatagtgaacttttttcctg

SEQ ID NO: 1065 - DNA, 60 bp, Eucalyptus camaldulensis
ttttttgggtgtgctccactgtttgctttgtgtttgtcctgaaaattgaagcaactgtat

SEQ ID NO: 1066 - DNA, 60 bp, Eucalyptus camaldulensis
ttttccaggatggcaattgttcgagaatcaaccttgtttactgatcacaatgttgccatg

SEQ ID NO: 1067 - DNA, 60 bp, Eucalyptus camaldulensis
agacatgagagatgcaattattgttcatgtgttcattgtctgcgttgtatatgatcgtcg

SEQ ID NO: 1068 - DNA, 60 bp, Eucalyptus camaldulensis
gtaactttctatattgtaacagatgtatagtagctaggacatcattatttggtgtgggac

SEQ ID NO: 1069 - DNA, 60 bp, Eucalyptus camaldulensis
gaaggacgtctcccctcatgagtttgtcaagcctacgccgcgcatctcaagcgttccggg

SEQ ID NO: 1070 - DNA, 60 bp, Eucalyptus camaldulensis
tctctgtgtgagcgtgcatggaaaaatgtaacgaacggatttgtcagcatggtgttccta

SEQ ID NO: 1071 - DNA, 60 bp, Eucalyptus camaldulensis
acatccaaggactgtcaacattgtccaccatgtcgtgcctattgtgccattttcagacaa

SEQ ID NO: 1072 - DNA, 60 bp, Eucalyptus camaldulensis
aaccttgtgtaacagaggaaattacatgagattcctccattctggccgttttctttttta

SEQ ID NO: 1073 - DNA, 60 bp, Eucalyptus camaldulensis
ggtcgttgccttcctttgtggaaaacgggattcggaagtttcgatgttgacgtgataaaa

SEQ ID NO: 1074 - DNA, 60 bp, Eucalyptus camaldulensis
tgtgttttcaagaggggtgggtgagggttctcttttttgacagtgacccaacaacaaact

SEQ ID NO: 1075 - DNA, 60 bp, Eucalyptus camaldulensis
gtagataggcttaatttctagcggattagggtttaggacttagtttggccgttccttttt

SEQ ID NO: 1076 - DNA, 60 bp, Eucalyptus camaldulensis
cggcccaggggtgtcctcgttgtatctttagctattcatattttgactttgataagccta

SEQ ID NO: 1077 - DNA, 60 bp, Eucalyptus camaldulensis
cttttctggctttatgaccggaaatatcaaaagctagcatataaactcgtcgcttgtcct

SEQ ID NO: 1078 - DNA, 60 bp, Eucalyptus camaldulensis
ctcctttggattctgtagctatctttgtggatcttattagcaagcttttcccgtggtatt

SEQ ID NO: 1079 - DNA, 60 bp, Eucalyptus camaldulensis
ttttgctgatgtttggttggagaaaaaactgtcgtcgcctgtcattcattttgggcctca

SEQ ID NO: 1080 - DNA, 60 bp, Eucalyptus camaldulensis
gaatgttttctggagagaggagaattcagatgtcagtgagagctcatattgaatttgagc

SEQ ID NO: 1081 - DNA, 60 bp, Eucalyptus camaldulensis
tgtttgggctttgtcttgtaccggttgtatcttgggtgtttaataatattatcctctgat

SEQ ID NO: 1082 - DNA, 60 bp, Eucalyptus camaldulensis
ttttttgagttccaacgatataacgagggaggaccgttatgtttttctttatggagtaat

SEQ ID NO: 1083 - DNA, 60 bp, Eucalyptus camaldulensis
tcttggtctctttgcatttatgttgaggaatatagagagctgcaatgctattgaattaca

SEQ ID NO: 1084 - DNA, 60 bp, Eucalyptus camaldulensis
ttcggatggtaatttctttagttctaatttgtaacttgactgcacctatttgaagcaagc

SEQ ID NO: 1085 - DNA, 60 bp, Eucalyptus camaldulensis
tggtcgttaacccgacgattgtagtttcagtattgtgaaatctaatgctggattgattct

SEQ ID NO: 1086 - DNA, 60 bp, Eucalyptus camaldulensis
ttctctgttattggtaacagagagaactgttctttgtttccattgaaggggtgaagttgg

SEQ ID NO: 1087 - DNA, 60 bp, Eucalyptus camaldulensis
ttttattgcgtcgttcagttcttttatgagcgtagggtgtatatatagagagatcggttt

SEQ ID NO: 1088 - DNA, 60 bp, Eucalyptus camaldulensis
tcttgcagggttattatcttatgtatcctgaggtacaatacacagtcttaggtggcttgc

SEQ ID NO: 1089 - DNA, 60 bp, Eucalyptus camaldulensis
aagtggaaaccttggtgaaccctggattatgcccaatttgatgttcaatgacactcggga

SEQ ID NO: 1090 - DNA, 60 bp, Eucalyptus camaldulensis
gtgtatgaattggatctatattgagtgaatagaatttcatgctttaaaaagatatttttg

SEQ ID NO: 1091 - DNA, 60 bp, Eucalyptus camaldulensis
agaacacatctagatgccccaatctcatctttcaaggtttttaagatatgtggattggat

SEQ ID NO: 1092 - DNA, 60 bp, Eucalyptus camaldulensis
aatgtatatcatagttgatcgtcttgaatgcgattagcatgttattcgccccttcgttct

SEQ ID NO: 1093 - DNA, 60 bp, Eucalyptus camaldulensis
tctatgcggcaaattttgggttgggaggaaactttgttttaaatggtaatagcccctttg

SEQ ID NO: 1094 - DNA, 60 bp, Eucalyptus camaldulensis
tttacatgtaagcagttgtgacactgggcgcgaatttgggatctagaactttgtatctga

SEQ ID NO: 1095 - DNA, 60 bp, Eucalyptus camaldulensis
cttttatttctgacttgtgctatatgaatgccatgttgttctgtttgcatttaaaaaaaa

SEQ ID NO: 1096 - DNA, 60 bp, Eucalyptus camaldulensis
tgcagctcaggtgtttgtggaatttactgaacgagcttctgaattagattgcaggcgctt

SEQ ID NO: 1097 - DNA, 60 bp, Eucalyptus camaldulensis
aaacagaatgccgtagtgttttctccgttgtggtgctgtttatttgtctcagtgtaagaa

SEQ ID NO: 1098 - DNA, 60 bp, Eucalyptus camaldulensis
agttgtgaatttcctgcttttggagtaatgatcaatgaaggaatggactggttctactaa

SEQ ID NO: 1099 - DNA, 60 bp, Eucalyptus camaldulensis
ttaaatctcagtgtgttatggtttttatggcaggttaatgaaagccgatgtttttgctca

SEQ ID NO: 1100 - DNA, 60 bp, Eucalyptus camaldulensis
agaggaacgtgcaagcccagtgggaatatcactttcttgtatgaatgtgaatgtgaccct

SEQ ID NO: 1101 - DNA, 60 bp, Eucalyptus camaldulensis
aggtaggtcagtttcgtgaattctactcaacttgtaaatagcgttctcatcttgttttcc

SEQ ID NO: 1102 - DNA, 60 bp, Eucalyptus camaldulensis
acactccggccttgcgatcaaccttaatgtgctcagttagtagctttacgattggatgat

SEQ ID NO: 1103 - DNA, 60 bp, Eucalyptus camaldulensis
aaatggtttgtcgtccctttgtaaccgaagggcaggatccggatttcgatggtgtattta

SEQ ID NO: 1104 - DNA, 60 bp, Eucalyptus camaldulensis
ttcgctgctccctgcattgtagttgttgttttctgcatgacgccgtgaaattctattgga

SEQ ID NO: 1105 - DNA, 60 bp, Eucalyptus camaldulensis
cagatgatgaatgataaaggcgattgaaaggtctgttcttgcggtcaaaaagttggcact

SEQ ID NO: 1106 - DNA, 60 bp, Eucalyptus camaldulensis
ggaggatttttcaactgtgtaataagcattctgctatgtgatttgaagagcgaggaatga

SEQ ID NO: 1107 - DNA, 60 bp, Eucalyptus camaldulensis
ccagctgaatctcaaggacatcattgacttctatgctcaagatttttagagagacgagga

SEQ ID NO: 1108 - DNA, 60 bp, Eucalyptus camaldulensis
tggttctactgctgttctttcggttcaattaccatctgaagcaaccaggcgactgatgtt

SEQ ID NO: 1109 - DNA, 60 bp, Eucalyptus camaldulensis
gtgaaagagtaatttgatgtgctatgctttcgtttttacttctctttgtcttataggcag

SEQ ID NO: 1110 - DNA, 60 bp, Eucalyptus camaldulensis
gttagtgaagtaaagcggatcaacgtttaagagataatcagtttcattaagcaagcaatg

SEQ ID NO: 1111 - DNA, 60 bp, Eucalyptus camaldulensis
acctgatcttgggacattgaagcatcctatctattagtatgagaaagtgcttgtttgtgt

SEQ ID NO: 1112 - DNA, 60 bp, Eucalyptus camaldulensis
cacaccagacctgaactcttgtcgttttcaataattttcaggctggatcttttttgagtt

SEQ ID NO: 1113 - DNA, 60 bp, Eucalyptus camaldulensis
tcagacaattagagtttttgcttgtaagctctatcttcaaagtcaatggcttcaagtcct

SEQ ID NO: 1114 - DNA, 60 bp, Eucalyptus camaldulensis
gatgtcctgcttatcttcatgtaggatgtttcagctgtgacaattagcatgttttttaag

SEQ ID NO: 1115 - DNA, 60 bp, Eucalyptus camaldulensis
tcagtccccattgatgtatcagcttcttaattaatcttgctagtctgtctctgtttcgtc

SEQ ID NO: 1116 - DNA, 60 bp, Eucalyptus camaldulensis
ctagagctttgctgatcagattgctgatgctcaatgattaaatcctttgaccatattccg

SEQ ID NO: 1117 - DNA, 60 bp, Eucalyptus camaldulensis
gtaattcttgcgacgaggttggtagtctctgactttctgatgtacaatgaaatgcttatc

SEQ ID NO: 1118 - DNA, 60 bp, Eucalyptus camaldulensis
tctggtctgttagcacatgataaagatggacaccgagatgcacttaggtcaaagtgatgg

SEQ ID NO: 1119 - DNA, 60 bp, Eucalyptus camaldulensis
ccaaagcgcgagctaatcctcattcagcatttatgattttctcaccacattccatttttg

SEQ ID NO: 1120 - DNA, 60 bp, Eucalyptus camaldulensis
tcttgagaggttggatgcttttgtttgtattgactgattgatccttattctggttgatta

SEQ ID NO: 1121 - DNA, 60 bp, Eucalyptus camaldulensis
aaatagttgatgagttcaaaatcccaattctgtttttctatttcgattggcgttctcagg

SEQ ID NO: 1122 - DNA, 60 bp, Eucalyptus camaldulensis
gaggaacaagtcgacaaatttgatgagcttattcatcttatttgtgtatgataccacaca

SEQ ID NO: 1123 - DNA, 60 bp, Eucalyptus camaldulensis
ttaacttcaagaaagacaaggatggctactggaagtggtttggtggtaacttggcatcag

SEQ ID NO: 1124 - DNA, 60 bp, Eucalyptus camaldulensis
atggcactagtcgacggaaattatctttgttttttacctttgatttcgtcataatcaagg

SEQ ID NO: 1125 - DNA, 60 bp, Eucalyptus camaldulensis
ttgattgtacttctcaccatctgttttttccctatcatacattagaactagtatctgtgg

SEQ ID NO: 1126 - DNA, 60 bp, Eucalyptus camaldulensis
tgcttgtacatattaacttcttagggggaacagtgaataaataaataagttgtttttgca

SEQ ID NO: 1127 - DNA, 60 bp, Eucalyptus camaldulensis
gatggccgtgaaattccaattgtgcatcgtgtaataaaggtacatgaacgccaagatact

SEQ ID NO: 1128 - DNA, 60 bp, Eucalyptus camaldulensis
acctgacagtgttcgtttcattcttgtcactcttttgccccagtttgcagggcttgtaat

SEQ ID NO: 1129 - DNA, 60 bp, Eucalyptus camaldulensis
ttgaggacttagtgaagattttgtgattatgtggacatgttggcttttctattacatcga

SEQ ID NO: 1130 - DNA, 60 bp, Eucalyptus camaldulensis
tgtaacagctgtgcacttgccgtctctgtaatgacataataaataaagtctaaagatgat

SEQ ID NO: 1131 - DNA, 60 bp, Eucalyptus camaldulensis
atgattcagacgagaattagaattttctgcagtatctctctgtattgtttcacttggtct

SEQ ID NO: 1132 - DNA, 60 bp, Eucalyptus camaldulensis
tggcaagtaaattaaagaaccaagataaccatccggaaatagtcggatgttttttgttcg

SEQ ID NO: 1133 - DNA, 60 bp, Eucalyptus camaldulensis
agtgaaagtgtgcctgacctagcatcaagaagcatagaatcttgtcaagtaaatcagctt

SEQ ID NO: 1134 - DNA, 60 bp, Eucalyptus camaldulensis
gcatgtgcatcttggtgaccttatagaccatttgggggacatttgaagatatttgacctt

SEQ ID NO: 1135 - DNA, 60 bp, Eucalyptus camaldulensis
atgatctgtataacgatttgcggtctacatgtgttgtttgcttgttgaggggcgagcact

SEQ ID NO: 1136 - DNA, 60 bp, Eucalyptus camaldulensis
catttaaaacctttctccttcctgttttattactgtatcctgttggaattgtctaatggg

SEQ ID NO: 1137 - DNA, 60 bp, Eucalyptus camaldulensis
ctttgatatagagatgaacttgtgcccgggaaattcaggtttcagtttttcgagatatct

SEQ ID NO: 1138 - DNA, 60 bp, Eucalyptus camaldulensis
ggtagcttgtattgctctgctcaaggtgctaatggataagtcattctgttggagcaaaaa

SEQ ID NO: 1139 - DNA, 60 bp, Eucalyptus camaldulensis
tcaaggagttccatctctttcaaacttgttgcctggagcggcaaggagcaactaaatgtt

SEQ ID NO: 1140 - DNA, 60 bp, Eucalyptus camaldulensis
tgcgttttgttgtgaagattacacatgatgagcaatatgaattcattttcctccttttgc

SEQ ID NO: 1141 - DNA, 60 bp, Eucalyptus camaldulensis
tatgatgtatctctgcggccactgttcgagatatccatcatctttaaaattttcgaagtc

SEQ ID NO: 1142 - DNA, 60 bp, Eucalyptus camaldulensis
ggttcctctgccttttgcctcgagttttacattgaatattaggattctttcaacttggcc

SEQ ID NO: 1143 - DNA, 60 bp, Eucalyptus camaldulensis
tgctgcaaataatatcagatccgcctcaaggaagtgaaaatcttttgatgctggtgctcc

SEQ ID NO: 1144 - DNA, 60 bp, Eucalyptus camaldulensis
cagattgtgaggaacttcatcttgagagtttgtctgtgaacttgtattggcgagtagagt

SEQ ID NO: 1145 - DNA, 60 bp, Eucalyptus camaldulensis
gctcttatatcttggtgggtttttcgctatggattacgaaagagcagtttatcacccagt

SEQ ID NO: 1146 - DNA, 60 bp, Eucalyptus camaldulensis
ttcctgacatttatatccgaatgccttttctcttgagtttctttaacgcatctatctgag

SEQ ID NO: 1147 - DNA, 60 bp, Eucalyptus camaldulensis
gaactcgttcttgaatatgctgcttgtaagagatttgaacttttctgaactgcatatgta

SEQ ID NO: 1148 - DNA, 60 bp, Eucalyptus camaldulensis
tcttggggcgggattgatgtctttgctagccatctgggcttgatttcattaagatagctt

SEQ ID NO: 1149 - DNA, 60 bp, Eucalyptus camaldulensis
tgtaatttgtctagtggacggtgtgaggtaaaggcagttctttgtaatgtctctctttgc

SEQ ID NO: 1150 - DNA, 60 bp, Eucalyptus camaldulensis
aaaggagcaacttcatagactttcatgtctcaaggcggaggtcaagtccttcatcctctt

SEQ ID NO: 1151 - DNA, 60 bp, Eucalyptus camaldulensis
aaacacgtcacgtgcttttaaatgtttggtgctgctaatgaaatcaaactttctggtggg

SEQ ID NO: 1152 - DNA, 60 bp, Eucalyptus camaldulensis
cttgatcttattcttgttgttgggtcgttttgtgaggatttgtagaaagatttgacctaa

SEQ ID NO: 1153 - DNA, 60 bp, Eucalyptus camaldulensis
atgcaacctgatcggtatgtaaaatcaagatgtggattgcttcaatatgtgcttacttct

SEQ ID NO: 1154 - DNA, 60 bp, Eucalyptus camaldulensis
actggataagatatgtctttaaattgacggggtttttgagcctataggctatatacgtgc

SEQ ID NO: 1155 - DNA, 60 bp, Eucalyptus camaldulensis
cttgatgacagttccttttcttccccttatatatgttatgatggattggagtgttctttt

SEQ ID NO: 1156 - DNA, 60 bp, Eucalyptus camaldulensis
gctaatgtgtagtggcttcttatatgtagtgttgactctggagatataatgaatgaagga

SEQ ID NO: 1157 - DNA, 60 bp, Eucalyptus camaldulensis
ccgaagcagcatctgctggcagcaattgacgatccttgtgttcaggttgtaatttgtttt

SEQ ID NO: 1158 - DNA, 60 bp, Eucalyptus camaldulensis
tcgctctgtaccgtgttaattttctgaacatcttgcgcctttaggcgacgaatcttcaat

SEQ ID NO: 1159 - DNA, 60 bp, Eucalyptus camaldulensis
gtcatgctcattttctggattttacccagctaatcatgacttctagaactctatcgcatg

SEQ ID NO: 1160 - DNA, 60 bp, Eucalyptus camaldulensis
tatacagagcgtaggcttgctttgagcactctaatttcttcaaagtaacagcaccggagg

SEQ ID NO: 1161 - DNA, 60 bp, Eucalyptus camaldulensis
cttggtctcaatgttgcttgtgagaaggttcccctacccaagtaaagattggctacttct

SEQ ID NO: 1162 - DNA, 60 bp, Eucalyptus camaldulensis
aacgttagactaccttccaccggaaatggtggagagtgttgagcatgatgcaaatgtaga

SEQ ID NO: 1163 - DNA, 60 bp, Eucalyptus camaldulensis
cctgtttgagaaatacatccctgactatttcatgtttccaaatatcttacggaatctatc

SEQ ID NO: 1164 - DNA, 60 bp, Eucalyptus camaldulensis
ggccattatggccggggatgaggattgtcaagtcttcatatattaagttgatatgtaaag

SEQ ID NO: 1165 - DNA, 60 bp, Eucalyptus camaldulensis
ccatggccatggccatgggaagttcaagcatgggaaatttggcaagcacggcaagcatgg

SEQ ID NO: 1166 - DNA, 60 bp, Eucalyptus camaldulensis
attactcgcgatttcattttgtttatatcgaatatcaaacgtgtaatctcgtcaccagat

SEQ ID NO: 1167 - DNA, 60 bp, Eucalyptus camaldulensis
tggttcttgaatcttgaatctgctgcctgtgaatgcttcccagagaagatgagcatgtgt

SEQ ID NO: 1168 - DNA, 60 bp, Eucalyptus camaldulensis
gtcattataattccagatcgagacctggccaagtactggaataatgtctggagaggtatg

SEQ ID NO: 1169 - DNA, 60 bp, Eucalyptus camaldulensis
ttctatgctctttcatgaattgtaacattggctaaaacagatctcttgtatcttttcccc

SEQ ID NO: 1170 - DNA, 60 bp, Eucalyptus camaldulensis
tccctggtagtggaagttgagaatttatgtgattatatgttgcttccaggtttcagctct

SEQ ID NO: 1171 - DNA, 60 bp, Eucalyptus camaldulensis
gagtatctatagatgagtgttggtattctcctattgttcttcttccgtgaatgtttaagg

SEQ ID NO: 1172 - DNA, 60 bp, Eucalyptus camaldulensis
tggtcgattttcggttctcttttggccgaacctacaaattttatgacatggtgaattcgt

SEQ ID NO: 1173 - DNA, 60 bp, Eucalyptus camaldulensis
agatcacttagacaggaatttgattactcgattgttctgaatcgaacacaaatgttcagt

SEQ ID NO: 1174 - DNA, 60 bp, Eucalyptus camaldulensis
caggtcgacgagcttaacgtgtgtaatattattgtttaatgggaatcctaaggttgtgaa

SEQ ID NO: 1175 - DNA, 60 bp, Eucalyptus camaldulensis
aaacctcgctattgctatatacaagatcgaccctccgtccactcaagaatgaagggttgt

SEQ ID NO: 1176 - DNA, 60 bp, Eucalyptus camaldulensis
ttttggttgtaattggcggatggaactcttacttttgactttgaggaggatgagacgtgt

SEQ ID NO: 1177 - DNA, 60 bp, Eucalyptus camaldulensis
cagttaggactccctacttactagatgaatttgctagttttggatgggaattttatgtta

SEQ ID NO: 1178 - DNA, 60 bp, Eucalyptus camaldulensis
gtgaccttatagaccatttggtgtggacatttgaagatatttgaccttaaatgtactttc

SEQ ID NO: 1179 - DNA, 60 bp, Eucalyptus camaldulensis
cggaagaatgtgctcatttgacgatgttgatgcttatttcttttcatttgattgtattgc

SEQ ID NO: 1180 - DNA, 60 bp, Eucalyptus camaldulensis
tgtcactgtagcaggtgggggctgatctggtcttctgacctgattgtgttaaataagatt

SEQ ID NO: 1181 - DNA, 60 bp, Eucalyptus camaldulensis
agttgacatatcaattttggtgaaccgtgcccggttatgatgactgtgtttgaaagacat

SEQ ID NO: 1182 - DNA, 60 bp, Eucalyptus camaldulensis
tgtgtttctggttcaccattccttttctgcattgtactcgcgcacccgtgtttgagtaaa

SEQ ID NO: 1183 - DNA, 60 bp, Eucalyptus camaldulensis
atatctctgtgttacccattttcggaacagtaatctgtaagtgcaatttgtactcttctc

SEQ ID NO: 1184 - DNA, 60 bp, Eucalyptus camaldulensis
ttgttgaagtgttatgtgtctttctcgtctccaatcagtgcaaatggagataatctgttc

SEQ ID NO: 1185 - DNA, 60 bp, Eucalyptus camaldulensis
tgatagatgaaccaatttatccttcgagaggcatggcacgaaccactgctaattttgtgt

SEQ ID NO: 1186 - DNA, 60 bp, Eucalyptus camaldulensis
tgattgtttttgtgcgtgatcattatatctgtacacgatcttccaacattccaactttag

SEQ ID NO: 1187 - DNA, 60 bp, Eucalyptus camaldulensis
atcgatgacagtgatatcctactttggtggacttataatttgctgagagactttggctat

SEQ ID NO: 1188 - DNA, 60 bp, Eucalyptus camaldulensis
atgtaggggtgattgtaattggatcgtcatctatgacgaacattttaatctattaactcg

SEQ ID NO: 1189 - DNA, 60 bp, Eucalyptus camaldulensis
actggagcaacggcctattgaattgtatcatgtggttgtattgaacaagcttttaatcaa

SEQ ID NO: 1190 - DNA, 60 bp, Eucalyptus camaldulensis
ctcgtaccacgcgtaatcctttagagggtgttctgagtgagttatttaagtttcatgctt

SEQ ID NO: 1191 - DNA, 60 bp, Eucalyptus camaldulensis
ttgctccttctttggaagcttggaaattctacaaatccaccatcgatgaatggactgcac

SEQ ID NO: 1192 - DNA, 60 bp, Eucalyptus camaldulensis
ttatgtttgccaaaggacgcgttcaccacgcattttttgccggttaataaagcatataaa

SEQ ID NO: 1193 - DNA, 60 bp, Eucalyptus camaldulensis
tcgcagcttcgcggtatgtggacttctcataaatgttcacttttgtatttttatttgatc

SEQ ID NO: 1194 - DNA, 60 bp, Eucalyptus camaldulensis
atttttgctattttcaaatagcatcatatactatcaaactctcatgtgaatttgagaatt

SEQ ID NO: 1195 - DNA, 60 bp, Eucalyptus camaldulensis
acatagggttaagtttgataaaacgcattctttgtgtaatctctgtatttcagtctgctc

SEQ ID NO: 1196 - DNA, 60 bp, Eucalyptus camaldulensis
tacctgaaatgcgtttccaaaatctctctccgcagcctccagacccattttgtttgtgta

SEQ ID NO: 1197 - DNA, 60 bp, Eucalyptus camaldulensis
tgtgagaaaggcactggaaagagtggaaaacctcttcactacaaggtgaagcagtttcca

SEQ ID NO: 1198 - DNA, 60 bp, Eucalyptus camaldulensis
acttgatttgattctagcatatgcagaacacaaaatgcagtctgcgttttataatggcca

SEQ ID NO: 1199 - DNA, 60 bp, Eucalyptus camaldulensis
attcactggctcgtacagtgttgggaagtcttaagtttatcaacattatgctgcttgaat

SEQ ID NO: 1200 - DNA, 60 bp, Eucalyptus camaldulensis
taatttacttgtgtgggaatctttcctgtttcgagcatcagatgtaaatctgtgaatgat

SEQ ID NO: 1201 - DNA, 60 bp, Eucalyptus camaldulensis
caagattgacagccgtgtaccacctgtggtcaaacaagtttccgcccaggccatgtctgc

SEQ ID NO: 1202 - DNA, 60 bp, Eucalyptus camaldulensis
ttgtgagttttatttatgtgatgcggcttctctcctgaccgttccggggagaaacttgat

SEQ ID NO: 1203 - DNA, 60 bp, Eucalyptus camaldulensis
aatttactccactgtagagctagtcctagtttaattggcaatattgctggttacgcgttg

SEQ ID NO: 1204 - DNA, 60 bp, Eucalyptus camaldulensis
aacacagtactctggtgtatttgaagtcgatgactttcttatgtactcgagaaccattat

SEQ ID NO: 1205 - DNA, 60 bp, Eucalyptus camaldulensis
aatgctgtcagttgcatctcgatgtgaacagcattgtacttttgatattggggttatatt

SEQ ID NO: 1206 - DNA, 60 bp, Eucalyptus camaldulensis
ccctttcaccaaacatccccatttcttcggagggctcttgacttctggaattcttttgtt

SEQ ID NO: 1207 - DNA, 60 bp, Eucalyptus camaldulensis
ctggagtgtaatttggggttgaaaaatagccaaaattgcagatgcagtgttggggtgatc

SEQ ID NO: 1208 - DNA, 60 bp, Eucalyptus camaldulensis
ttttatggaactcggtttcaattttatgtgaaggctgcctagtgaaaatctcagtataag

SEQ ID NO: 1209 - DNA, 60 bp, Eucalyptus camaldulensis
aatcagtgatgagtcaggattgatatctctgtacttcggttttttatcttctgctcattt

SEQ ID NO: 1210 - DNA, 60 bp, Eucalyptus camaldulensis
ggtactcgatgagattcttttcttgctgtagtattggtccattcttgcttcttgtaagtg

SEQ ID NO: 1211 - DNA, 60 bp, Eucalyptus camaldulensis
agagcttcaaagcgcacacaaccctcaacactaaacaaaagcacgatttggcaaatcggt

SEQ ID NO: 1212 - DNA, 60 bp, Eucalyptus camaldulensis
agttcttccagtccaagtactgcaagagtaaatcggtcatcacattccacttcccagcaa

SEQ ID NO: 1213 - DNA, 60 bp, Eucalyptus camaldulensis
aaatgagaaatactccggtggcctactctttaaattttgattcattctgcttctggcatt

SEQ ID NO: 1214 - DNA, 60 bp, Eucalyptus camaldulensis
cttgaccatctggcatctgccatgtattttcctttcttaaccaaagacactttgtgaaaa

SEQ ID NO: 1215 - DNA, 60 bp, Eucalyptus camaldulensis
ttagtaccgtcatgtaaaacagtcagatcgagaaggaggcttgaaccttgattcaagcag

SEQ ID NO: 1216 - DNA, 60 bp, Eucalyptus camaldulensis
gagcccttgcacgttctggcatgaaaatcggtcgcatagaggatgtgacaccgataccaa

SEQ ID NO: 1217 - DNA, 60 bp, Eucalyptus camaldulensis
aattcaactgatatttagccaaaattgatggccacgagcgtgggaaaacctgaatcttag

SEQ ID NO: 1218 - DNA, 60 bp, Eucalyptus camaldulensis
agatgaagtggacaaattgcagaaatacttggcttgagtcagctgcctcccttgtggaat

SEQ ID NO: 1219 - DNA, 60 bp, Eucalyptus camaldulensis
ttctcaccaaatgggacttccatttcaccagatggagctcattttgcatgtaaaatacgt

SEQ ID NO: 1220 - DNA, 60 bp, Eucalyptus camaldulensis
tctgtggagatgtattaggataattggtagtgactgttcactgcagacttcttcatagtt

SEQ ID NO: 1221 - DNA, 60 bp, Eucalyptus camaldulensis
ttatctgtttcaagacgacaattgttccaattttcttagctcgagatggactgtgcaaac

SEQ ID NO: 1222 - DNA, 60 bp, Eucalyptus camaldulensis
agcatactgtgagatacaacaacgtaggagcagctacaatgagagcccccttagatcatt

SEQ ID NO: 1223 - DNA, 60 bp, Eucalyptus camaldulensis
tcatcgcgctggaatttggacttccttgaatacctattgggacttgaatatgttaacatt

SEQ ID NO: 1224 - DNA, 60 bp, Eucalyptus camaldulensis
gagagctcaacttcgttatcgcaggagacttttgtgtggaccattatactccttttgtta

SEQ ID NO: 1225 - DNA, 60 bp, Eucalyptus camaldulensis
cctttggcatggcagtaggatagctgcataagttagttatttctctgttctgattcatgc

SEQ ID NO: 1226 - DNA, 60 bp, Eucalyptus camaldulensis
catgcagacttgtcattgttgcttaatactccaataattgagacatccttatataagagc

SEQ ID NO: 1227 - DNA, 60 bp, Eucalyptus camaldulensis
taccggattacttcgagtgacttgagttggaagcgtctccctcccatcaatttatccatt

SEQ ID NO: 1228 - DNA, 60 bp, Eucalyptus camaldulensis
tgattggtttagctgagtttgtatatgcatattcgctatgaacatgcccaagaaggtgct

SEQ ID NO: 1229 - DNA, 60 bp, Eucalyptus camaldulensis
ttgactgatttaaagtcgccatacttgcggccacgatgatatttgatcttgatttgggga

SEQ ID NO: 1230 - DNA, 60 bp, Eucalyptus camaldulensis
ttgtacttaaatttgctggttgacctctgcattttcttatgtgaagtaccgttacctgag

SEQ ID NO: 1231 - DNA, 60 bp, Eucalyptus camaldulensis
tactctgcctatacttcgcgtatataggattttgaatcgttttccacaatgttaaactga

SEQ ID NO: 1232 - DNA, 60 bp, Eucalyptus camaldulensis
gcaccgatcagctttggtctcgatggctttggttagtcaagagctttaagattttgaatg

SEQ ID NO: 1233 - DNA, 60 bp, Eucalyptus camaldulensis
tggctaccgaagtatgactttaggcaattgctagaacatttattctgtcttacaactctt

SEQ ID NO: 1234 - DNA, 60 bp, Eucalyptus camaldulensis
acggggaacttgacttcgctctccgagcaagagctcatcgactgtgacacaacttacaac

SEQ ID NO: 1235 - DNA, 60 bp, Eucalyptus camaldulensis
ctgaaaccatttcatgacaatgtgtaatgtctagctatataaaatggatctcgtgccttt

SEQ ID NO: 1236 - DNA, 60 bp, Eucalyptus camaldulensis
tgttgtcttcaccttgtgggactaaagttgagtacaaaataccatttctgcatttcgact

SEQ ID NO: 1237 - DNA, 60 bp, Eucalyptus camaldulensis
gtgtggatagacttagacacgttctaagcaactctatgaacgtgataatggctttttcgc

SEQ ID NO: 1238 - DNA, 60 bp, Eucalyptus camaldulensis
agattcttgacgacaagtgttagtggtctggcagctgtaagttatggcatgtatcgcatc

SEQ ID NO: 1239 - DNA, 60 bp, Eucalyptus camaldulensis
ctagtaagagcttctgtatttcccttctctgcatttaccactagaatgttcaagtatttt

SEQ ID NO: 1240 - DNA, 60 bp, Eucalyptus camaldulensis
aacattgttgagctggggacataaacacatgatgaaatatgctaccttagtgtatgctgg

SEQ ID NO: 1241 - DNA, 60 bp, Eucalyptus camaldulensis
attctggttgattgagtttgtaaattacttcctctctctgtccaagaattccagctgtta

SEQ ID NO: 1242 - DNA, 60 bp, Eucalyptus camaldulensis
cagacagtacatttgtatgttacatatcaaaattcatcaaatcgacagaaacttctccca

SEQ ID NO: 1243 - DNA, 60 bp, Eucalyptus camaldulensis
gtgtcgggttactattcagctgatgaccatctgtaacaagatgggcaaatctgccaaaga

SEQ ID NO: 1244 - DNA, 60 bp, Eucalyptus camaldulensis
atctctgtagaattgtgattcccaagcaaaaataacacatatcatgctactttgggagta

SEQ ID NO: 1245 - DNA, 60 bp, Eucalyptus camaldulensis
gccagcgctgctgctttttctgaaactgtgatggtttgtttcacacgccatagtatatgt

SEQ ID NO: 1246 - DNA, 60 bp, Eucalyptus camaldulensis
tgctcttcgcctggcatcttaatgtcctccgaagtcttaacattttaatgaaggaggtaa

SEQ ID NO: 1247 - DNA, 60 bp, Eucalyptus camaldulensis
acagtgccaagatgatgaggacgataattgtccgcagaaattaccttcatcttgttaaga

SEQ ID NO: 1248 - DNA, 60 bp, Eucalyptus camaldulensis
gctgattgttgtcaacacatgcaaccacactataatggaaactgtttcctgttgacatca

SEQ ID NO: 1249 - DNA, 60 bp, Eucalyptus camaldulensis
aaatagaagaaatgggaggctttgcttttgtgcctttaggaacaatctttccttcatctg

SEQ ID NO: 1250 - DNA, 60 bp, Eucalyptus camaldulensis
ttccaatgtgaacgctgcctcaattaattgtagctctcagaaaacatgactgctcgatgc

SEQ ID NO: 1251 - DNA, 60 bp, Eucalyptus camaldulensis
tcgacattctcgttttctgacagactaatgacacatggttaaatgtggtttatgactggg

SEQ ID NO: 1252 - DNA, 60 bp, Eucalyptus camaldulensis
atgggattgacccagattcttgcactaataccaggattgaagatgtacacatcgtctccg

SEQ ID NO: 1253 - DNA, 60 bp, Eucalyptus camaldulensis
gcatgtggacagtttttgattacctgaggatatattgttgttgccatctgttggatctca

SEQ ID NO: 1254 - DNA, 60 bp, Eucalyptus camaldulensis
tttgtagaccttttttgctttatctttagtggatggtccccagcttttctccagtgtgca

SEQ ID NO: 1255 - DNA, 60 bp, Eucalyptus camaldulensis
gtccctgcttcaattcaacgtaccatggtgataacgaataaagaaggcctgtttctgtcg

SEQ ID NO: 1256 - DNA, 60 bp, Eucalyptus camaldulensis
gcagtttgcagtttgaactcacgttcatgtgatcttattattgtaaccatacatttttgc

SEQ ID NO: 1257 - DNA, 60 bp, Eucalyptus camaldulensis
cctgattgatgaatctggaattttgagcgtatctattttaaactcttgaatagcaactgg

SEQ ID NO: 1258 - DNA, 60 bp, Eucalyptus camaldulensis
tggattgatgtataccttgacatcagtgtgaagctgcttgatttatgcaatgccttcagc

SEQ ID NO: 1259 - DNA, 60 bp, Eucalyptus camaldulensis
tcattatgccgggaatttttccgaaaaccggtcttcattttcattatgcatctttcttct

SEQ ID NO: 1260 - DNA, 60 bp, Eucalyptus camaldulensis
ctgtttgcctgattcaatggctgtatagtatttgatttgttagaattgcttatcgctcag

SEQ ID NO: 1261 - DNA, 60 bp, Eucalyptus camaldulensis
aacagaaagtactggcatgtatgtgccattatatgagagcatgttcatatatctcgtgat

SEQ ID NO: 1262 - DNA, 60 bp, Eucalyptus camaldulensis
tcaggcacttttccagcatcatgcatcatcaggccaggcatcgtcagtttgttctttttt

SEQ ID NO: 1263 - DNA, 60 bp, Eucalyptus camaldulensis
aggggttgagcttctgtgaattctatgctttaatgatcatatttgtttcatgagtggata

SEQ ID NO: 1264 - DNA, 60 bp, Eucalyptus camaldulensis
tcctggggcgtagtatttgtcgggcttgttgagcgaattgtcaaattttggagatcgttt

SEQ ID NO: 1265 - DNA, 60 bp, Eucalyptus camaldulensis
tgaggacatggatccagttgaggtttcgatttgatatatcgatgagtattttggcggaaa

SEQ ID NO: 1266 - DNA, 60 bp, Eucalyptus camaldulensis
aggcaggattgatgaaaagctatggaccacttactttcttgaaggttcataatgcggggc

SEQ ID NO: 1267 - DNA, 60 bp, Eucalyptus camaldulensis
aatttaaagaactacagccgaggatcataatttggggtcctgtaatagcagtcagtttgt

SEQ ID NO: 1268 - DNA, 60 bp, Eucalyptus camaldulensis
actgttccggagtcactttgagttgaagtatatgatgctgcgtcccatctggatgttcta

SEQ ID NO: 1269 - DNA, 60 bp, Eucalyptus camaldulensis
tttgaatcctgcgtagatattcctgtcaactgccattgtatcgtattgtgtcgtatgcac

SEQ ID NO: 1270 - DNA, 60 bp, Eucalyptus camaldulensis
ctagttggtgtgttgagctatacagtttgattaattgtgttcttgatgggtctgtgctga

SEQ ID NO: 1271 - DNA, 60 bp, Eucalyptus camaldulensis
ttgtctaccacaatgttcgaagggtttactttttggatggggattgaggactgatttcaa

SEQ ID NO: 1272 - DNA, 60 bp, Eucalyptus camaldulensis
agaccctgatcttgccatatgttaatcatgtgttgtgacacttggaaaatgtgtttttag

SEQ ID NO: 1273 - DNA, 60 bp, Eucalyptus camaldulensis
ttttttaaagaacatgcggatactcccttgacagatggtgcaataactttctgcatttcc

SEQ ID NO: 1274 - DNA, 60 bp, Eucalyptus camaldulensis
tgttttgccgaatcaaatatgagagccggcagagacatctctgtcatcatttttggccga

SEQ ID NO: 1275 - DNA, 60 bp, Eucalyptus camaldulensis
gtgtatgatttaacctggaatcaatcaataactagtgcatgagtgcttttcttggggtgg

SEQ ID NO: 1276 - DNA, 60 bp, Eucalyptus camaldulensis
tttcctttcccaaagtgagaccacaattttgaaatttatgcaactgctgttgaacatgca

SEQ ID NO: 1277 - DNA, 60 bp, Eucalyptus camaldulensis
agaaactactgcaggcgattctctctagcatatacactgtcacaactgtaatatatacgt

SEQ ID NO: 1278 - DNA, 60 bp, Eucalyptus camaldulensis
cagaatggcttcggaggctacaatcttttcaagaacaactgtgaggactttgctgtctac

SEQ ID NO: 1279 - DNA, 60 bp, Eucalyptus camaldulensis
ggggggatgggcagcttaatgaatacatgttgagtgtgtatcttagaacatatatggcac

SEQ ID NO: 1280 - DNA, 60 bp, Eucalyptus camaldulensis
ttaaatatattcaatcgcagtaatctgccctattgttgtcttagatctcaagatcacacc

SEQ ID NO: 1281 - DNA, 60 bp, Eucalyptus camaldulensis
tgaactcgcaactgagccaaaacgacgggcagataacgaccgtcatatttttctgatgta

SEQ ID NO: 1282 - DNA, 60 bp, Eucalyptus camaldulensis
gcattcactatccggcttgtcatctagttgaatctgatgactaagtgacatcgacttttg

SEQ ID NO: 1283 - DNA, 60 bp, Eucalyptus camaldulensis
gacagtgaagttacgcatcttagaaacttagcagctcaatgctcgtcattgggtcgtgtt

SEQ ID NO: 1284 - DNA, 60 bp, Eucalyptus camaldulensis
aacgattgtgcttgttctaactgtacagtgaaatgcatcagttgcttttctatacgcttt

SEQ ID NO: 1285 - DNA, 60 bp, Eucalyptus camaldulensis
ggctttatctattagttcgctgataagctgtacttaatcggatctgttctccatcaataa

SEQ ID NO: 1286 - DNA, 60 bp, Eucalyptus camaldulensis
tttgtatcagatcttggggtattaaactggtctatatgtataagtactgtgatgtggtag

SEQ ID NO: 1287 - DNA, 60 bp, Eucalyptus camaldulensis
caccgttatggtaccttgtctggtaagaatgccatgagtttctccctatttactgatcta

SEQ ID NO: 1288 - DNA, 60 bp, Eucalyptus camaldulensis
gggacatttatcaagtgattggcttttaattacgctctcggcttcaggttcgttggccat

SEQ ID NO: 1289 - DNA, 60 bp, Eucalyptus camaldulensis
gttcgtaaggatcacctatttgagttcgtaaaactctaaattttcttgttcattgcttgc

SEQ ID NO: 1290 - DNA, 60 bp, Eucalyptus camaldulensis
tactgtcttttgatgatgatttgagaagcaattgaaagcgcacgagttcagatccaatac

SEQ ID NO: 1291 - DNA, 60 bp, Eucalyptus camaldulensis
gatgcaactagagatgcacaggttctgaatactttacagatgtataaacatttgccctga

SEQ ID NO: 1292 - DNA, 60 bp, Eucalyptus camaldulensis
gtatctactttgtgatcgtggtgcattagtatttactggccagtggcatatgtacatgaa

SEQ ID NO: 1293 - DNA, 60 bp, Eucalyptus camaldulensis
ttgtttgtgatatctagaacacccagtggcaatttcggatgtggacgcaacgtagatgac

SEQ ID NO: 1294 - DNA, 60 bp, Eucalyptus camaldulensis
ggggggtcaatgactcaattctttcctttatgtaaagagcccgacggattctgagaatta

SEQ ID NO: 1295 - DNA, 60 bp, Eucalyptus camaldulensis
gccaaccaaaccaagagtccctggacacctgtgttgaaattaaggttgtatttttatggc

SEQ ID NO: 1296 - DNA, 60 bp, Eucalyptus camaldulensis
tgactgtactggtaatgcattttcattgatgttgtgatggttgaatgtttgcgacttctt

SEQ ID NO: 1297 - DNA, 60 bp, Eucalyptus camaldulensis
tttgtctctctctagttgggtcctttgggtttgtttgctatgggtcagttccgatgtgtt

SEQ ID NO: 1298 - DNA, 60 bp, Eucalyptus camaldulensis
cacttgagaaagatggcatttaaagagagtgcatgagatttttatgttgggggctgccta

SEQ ID NO: 1299 - DNA, 60 bp, Eucalyptus camaldulensis
tgttgctgtttcttttccatgaacctagaaacgggattcgcaattaaatgccaaattatg

SEQ ID NO: 1300 - DNA, 60 bp, Eucalyptus camaldulensis
catcttgaaatacttctcggcaccgtccggtcttattcagctgctttgcctttatggtta

SEQ ID NO: 1301 - DNA, 60 bp, Eucalyptus camaldulensis
aattttgctaaattcgagttgattgtagcattttagcaatcagattatttcctgcgcagg

SEQ ID NO: 1302 - DNA, 60 bp, Eucalyptus camaldulensis
acttttgaatgtgaatgcgtgagaaattaaacgtgtcctttactggaggaatgcagtgat

SEQ ID NO: 1303 - DNA, 60 bp, Eucalyptus camaldulensis
ttttgcttggccaaaaaacttgtgtgcttcaaaaagcttcagaaacagattactcggttc

SEQ ID NO: 1304 - DNA, 60 bp, Eucalyptus camaldulensis
tatttgaatctgggtatgcgtcaagctgcgatctgccgcggttttaggtgcagatcaagg

SEQ ID NO: 1305 - DNA, 60 bp, Eucalyptus camaldulensis
acaatgcttgcttgacttcaacaatcaaatttggtcttactctatcaatactatgatggg

SEQ ID NO: 1306 - DNA, 60 bp, Eucalyptus camaldulensis
ttggacttaaaaagatgctgtgttctcttccatcaattcaagtttgcattgatagcttgg

SEQ ID NO: 1307 - DNA, 60 bp, Eucalyptus camaldulensis
actgtcttttctgtcttcgcaacttcagtatgtccaagtggttcctccatttcgttgtcc

SEQ ID NO: 1308 - DNA, 60 bp, Eucalyptus camaldulensis
acctgtgaacctttctggccatcatcttgtttggattgagtaagaatctaagcttatttt

SEQ ID NO: 1309 - DNA, 60 bp, Eucalyptus camaldulensis
tgcattttggggtttgtacccatcaattcagtaaatggttcattttcttttcaacttgtc

SEQ ID NO: 1310 - DNA, 60 bp, Eucalyptus camaldulensis
tttcctaattgtgattctttccagatggatttatagtgtgtggaaagtaggttctcgaat

SEQ ID NO: 1311 - DNA, 60 bp, Eucalyptus camaldulensis
cttccaggctgcttttaacatcattttgagaaactctaatgcgaacatggttgaattcca

SEQ ID NO: 1312 - DNA, 60 bp, Eucalyptus camaldulensis
gcgatgaattgcgcattacatgcacacctgattgggaaaatctctgatgaaggccaagat

SEQ ID NO: 1313 - DNA, 60 bp, Eucalyptus camaldulensis
aagtggtcgtacaaaagggaaaagtgcaccttatcacttgctcttctaatggaactgagt

SEQ ID NO: 1314 - DNA, 60 bp, Eucalyptus camaldulensis
aattcaacaatggcttggtgtatcaaatgcggtacctaatgttatatttattgatgccag

SEQ ID NO: 1315 - DNA, 60 bp, Eucalyptus camaldulensis
cgcattgttcttcaacgtattaatggaaggtttttaaaacatattttgccgttcaatggg

SEQ ID NO: 1316 - DNA, 60 bp, Eucalyptus camaldulensis
tgctttttccctttggggctgagcagatgatgttgtttttgacctttgtaatggtagaaa

SEQ ID NO: 1317 - DNA, 60 bp, Eucalyptus camaldulensis
ctgcagttttgggattttgatactagttagcgtacacctgttcgtcggtgctttgtgttt

SEQ ID NO: 1318 - DNA, 60 bp, Eucalyptus camaldulensis
atgctgattttagctgaagcttgtttctctgatctcatatatttaagcttctttgcactc

SEQ ID NO: 1319 - DNA, 60 bp, Eucalyptus camaldulensis
taactggacttcgtgttgcaagttaatcatgtttccttatgtaaccaacgaatagttgat

SEQ ID NO: 1320 - DNA, 60 bp, Eucalyptus camaldulensis
tcacatgaatctgaaagattatccaggcttaagtggacccagctacgatgctgccaaaaa

SEQ ID NO: 1321 - DNA, 60 bp, Eucalyptus camaldulensis
atgagcataattatcatgaaacgctttcagtctatgcagtagaatcatggatgtggaaga

SEQ ID NO: 1322 - DNA, 60 bp, Eucalyptus camaldulensis
ccgcaatgtaaaaatacacttgggctatttcactcttcttatatcttccttcgatcagtc

SEQ ID NO: 1323 - DNA, 60 bp, Eucalyptus camaldulensis
gcatgcacgagcaaacgtgtgtagcattttatgttgaagctgtataatgcttctgtctaa

SEQ ID NO: 1324 - DNA, 60 bp, Eucalyptus camaldulensis
tggggtttctttcctttacgtgttacatctctctttcaagtaattaaacattcgagcaaa

SEQ ID NO: 1325 - DNA, 60 bp, Eucalyptus camaldulensis
ctctgtctcttgttgtaaaatacccactctttactgtataagaccctgaattgttgcgca

SEQ ID NO: 1326 - DNA, 60 bp, Eucalyptus camaldulensis
tgtgtacttggagatgagcctgacaggaattttttggaatgccattctttctggacagat

SEQ ID NO: 1327 - DNA, 60 bp, Eucalyptus camaldulensis
gagtgtttcccaaagggtatgactttgaatgaatggtttaaattatccatcgcagtgcat

SEQ ID NO: 1328 - DNA, 60 bp, Eucalyptus camaldulensis
ggtaggagttcgaattacagttgttattattaggtgtgcaaacatatcaaggctcggact

SEQ ID NO: 1329 - DNA, 60 bp, Eucalyptus camaldulensis
ctaataaaatttttgggcttgtgggggcactaagcacgccttcgtctcgtctaactttca

SEQ ID NO: 1330 - DNA, 60 bp, Eucalyptus camaldulensis
ggtttggggaacgttgcttgataattccagtgcagattgatatatatatgcagtaattga

SEQ ID NO: 1331 - DNA, 60 bp, Eucalyptus camaldulensis
tgtactatctgctttcctttgataccagattagttggtttttagcgtttatgcacggtcc

SEQ ID NO: 1332 - DNA, 60 bp, Eucalyptus camaldulensis
cggacatgaacagtatcctagtgctattcttcttgcgattgctttgagattttggtttcc

SEQ ID NO: 1333 - DNA, 60 bp, Eucalyptus camaldulensis
gaaaatgaacgattttgacgtcagcatggtttatcctgaaccatggtgcatgcctcgtct

SEQ ID NO: 1334 - DNA, 60 bp, Eucalyptus camaldulensis
gaatgaaattgactcaagctgtgtgatgaaaagtaccttccaacgtgtactatttgaata

SEQ ID NO: 1335 - DNA, 60 bp, Eucalyptus camaldulensis
ttttttatgtatcctgttttgtgattatttgcctgcgccagtatgtgctcatcttctagc

SEQ ID NO: 1336 - DNA, 60 bp, Eucalyptus camaldulensis
gctattcagataaatgatgcgattgatttgatctagaaaatgtcttcgttcaatctgctg

SEQ ID NO: 1337 - DNA, 60 bp, Eucalyptus camaldulensis
cttaaaatttgtacgagcattgttccgttgccgttgattgtcattaccgcaatagaacag

SEQ ID NO: 1338 - DNA, 60 bp, Eucalyptus camaldulensis
tttgaaccggctgccctgaacgttgaggtttttcagactgcagtctatttctgcttgtat

SEQ ID NO: 1339 - DNA, 60 bp, Eucalyptus camaldulensis
ttgtttttcctttttgggccatgttgtatggaatttgcttcgggagatccaagtcttggg

SEQ ID NO: 1340 - DNA, 60 bp, Eucalyptus camaldulensis
ttgatcttgtcatacacaaataaatatggaatgaagaaacctttccatcatcaagggacc

SEQ ID NO: 1341 - DNA, 60 bp, Eucalyptus camaldulensis
acgaagaatcaaaccaatgttgtacccatatgctttcttaataataacattgccatggtc

SEQ ID NO: 1342 - DNA, 60 bp, Eucalyptus camaldulensis
tctgcctctgtcgaatgactacttgaaatctgctgaacaagtggtgagtttctggcgaaa

SEQ ID NO: 1343 - DNA, 60 bp, Eucalyptus camaldulensis
tatattatatatttcacttcttgattaatgtataaaacgcctagatttttgcaacctgat

SEQ ID NO: 1344 - DNA, 60 bp, Eucalyptus camaldulensis
gactgcgaactgaatttcaagtttgtttgctgtaagatccctcaaatatgaaagtcgttt

SEQ ID NO: 1345 - DNA, 60 bp, Eucalyptus camaldulensis
cgcaaatttgcgtctccgcgtctacttgtgaatgtcgttgtaagttttgtaactaaccgg

SEQ ID NO: 1346 - DNA, 60 bp, Eucalyptus camaldulensis
cattcattgttgctgaaattatgtgtatagcttttgctcagaaatactgttggtttgagg

SEQ ID NO: 1347 - DNA, 60 bp, Eucalyptus camaldulensis
gctttgtgctcatggatattgatggcctgcgtgtggtggtctatgtttatgaactcattg

SEQ ID NO: 1348 - DNA, 60 bp, Eucalyptus camaldulensis
gacttttttcttttttatgtctcgagcggaatcagatagaatcttttgttgcaattgctg

SEQ ID NO: 1349 - DNA, 60 bp, Eucalyptus camaldulensis
caaccacggctattgctaccatatcgagcagatttgaatgggaattctgaagagtttagc

SEQ ID NO: 1350 - DNA, 60 bp, Eucalyptus camaldulensis
gatatttgtcaagagtgggcttttcaccctttctcagaagaacacaggctctgttgtgtt

SEQ ID NO: 1351 - DNA, 60 bp, Eucalyptus camaldulensis
ttttgactgcttaatatttgattctgtgtcctacattggaaaaatgaagcttgggtttgg

SEQ ID NO: 1352 - DNA, 60 bp, Eucalyptus camaldulensis
acaatcacctgaatttgtcattctaccttttaatctaaagctgcggcattagactgtttt

SEQ ID NO: 1353 - DNA, 60 bp, Eucalyptus camaldulensis
catgttgggtttggcagtcggaatcatttgtttgatgaatcatggtgttaggtggttgat

SEQ ID NO: 1354 - DNA, 60 bp, Eucalyptus camaldulensis
ttgcttttggctgttacatgttttccttagtccttgatggtaagtcgcagatggtttaga

SEQ ID NO: 1355 - DNA, 60 bp, Eucalyptus camaldulensis
tgcatgcttttcttgctgttgctgaagtgattgcatgagctcaggtttaaccctgattga

SEQ ID NO: 1356 - DNA, 60 bp, Eucalyptus camaldulensis
tgtttgccgtgagctgccaattgaaccatatactcctgaaaaccaggaatttttcaaggt

SEQ ID NO: 1357 - DNA, 60 bp, Eucalyptus camaldulensis
gaaggtttcgattgcctggggagagatcgccgcaattgcagcatctctgcgttggcctaa

SEQ ID NO: 1358 - DNA, 60 bp, Eucalyptus camaldulensis
aagtatgtggaccttggttcccgataatttgcgaggctattatacgattattgccgtgac

SEQ ID NO: 1359 - DNA, 60 bp, Eucalyptus camaldulensis
gttagtttctgtttcccagctggttatcaagttgttgatcatggatggctgagctttctc

SEQ ID NO: 1360 - DNA, 60 bp, Eucalyptus camaldulensis
cctctgatgttctgtaatgggacatattgattacttatatggtaagttttggtcagaatg

SEQ ID NO: 1361 - DNA, 60 bp, Eucalyptus camaldulensis
ttcttatgagcctttgaggacctcaaagttctccgagtaatttgagattagttctttaaa

SEQ ID NO: 1362 - DNA, 60 bp, Eucalyptus camaldulensis
gttgttcttcgttcgagttgtcgggcgtctgtcctacttgaggattcgaatattgaactt

SEQ ID NO: 1363 - DNA, 60 bp, Eucalyptus camaldulensis
ggtgaaattaatgccatgtagcttcatgtatgttgatcttttgagctgcttgcaccctgg

SEQ ID NO: 1364 - DNA, 60 bp, Eucalyptus camaldulensis
cgatatggacaacctacttcatgcaactttatttgtgttttatgacaatcttcccggttg

SEQ ID NO: 1365 - DNA, 60 bp, Eucalyptus camaldulensis
gaattctgtaattgataacagatcttgtacctattagctccactgggtattgtttgatcg

SEQ ID NO: 1366 - DNA, 60 bp, Eucalyptus camaldulensis
tacatttgcgtatgaccgtgcttttcctatttcttgtgggatttgcgtatgttgacttta

SEQ ID NO: 1367 - DNA, 60 bp, Eucalyptus camaldulensis
tgttgatatcacatgaaatcaaggaaatgcaggtattttttgttttttctttgggggagg

SEQ ID NO: 1368 - DNA, 60 bp, Eucalyptus camaldulensis
agaaacagcattttacgttcccggtcaatgtttgattgggctaatcgatgacttttgctt

SEQ ID NO: 1369 - DNA, 60 bp, Eucalyptus camaldulensis
acgttttgttgctagtttgagcttggaatcttggtcaaacattcttcttttccagatgag

SEQ ID NO: 1370 - DNA, 60 bp, Eucalyptus camaldulensis
acggatacttctttggtatatgctcagtatcatataagggtacatgtatatatccatgtg

SEQ ID NO: 1371 - DNA, 60 bp, Eucalyptus camaldulensis
ggctggtgtgcgcagtcaattctcatgcactcttgagacttatgaaatatctatgcatat

SEQ ID NO: 1372 - DNA, 60 bp, Eucalyptus camaldulensis
tttgaatattgcaagaagaagaatcacagcttgaactccagattctttctgcacgagttt

SEQ ID NO: 1373 - DNA, 60 bp, Eucalyptus camaldulensis
catgatgaggacatgcctttttaatgacctagtattgcgttgtatctcataaaatagttg

SEQ ID NO: 1374 - DNA, 60 bp, Eucalyptus camaldulensis
aagagtagtactagtgatgtggtgacctccttgtgaagtcctcgtgttgcacccctcttt

SEQ ID NO: 1375 - DNA, 60 bp, Eucalyptus camaldulensis
gaacatcaggcttaattcaattacatttattggagccttaactgcatgttgccacgtggg

SEQ ID NO: 1376 - DNA, 60 bp, Eucalyptus camaldulensis
gccgtctgtaaccatgcttttacatgctcacttttaagagttgaagatactgattagttc

SEQ ID NO: 1377 - DNA, 60 bp, Eucalyptus camaldulensis
gtagctgcaaacagcgtaaagaaattatctcaaccgatactgatctgttgaattgctaag

SEQ ID NO: 1378 - DNA, 60 bp, Eucalyptus camaldulensis
cccactcaaccacatgctttattgatcagcttaccactaaatgattgccactattttctt

SEQ ID NO: 1379 - DNA, 60 bp, Eucalyptus camaldulensis
aggatctccctttattgtaaaaccagatgtcttgctaatgttatcatgttaagcattcgc

SEQ ID NO: 1380 - DNA, 60 bp, Eucalyptus camaldulensis
ttttagacctgtagatcatatttggccttcgatggatgggttataaagtcgtgtcgtgat

SEQ ID NO: 1381 - DNA, 60 bp, Eucalyptus camaldulensis
tttcaccatctccgatgtcgtaactagcactttctttgttaatcgaactagtgactgttc

SEQ ID NO: 1382 - DNA, 60 bp, Eucalyptus camaldulensis
gaaagttcggcagctggcttctatatttatgaattgatcgcacatgatcaaactgtcgca

SEQ ID NO: 1383 - DNA, 60 bp, Eucalyptus camaldulensis
cgaagaacaatatcatgtcattgcgaacaaagtgcaagacgctgtatggcttgttgcgtg

SEQ ID NO: 1384 - DNA, 60 bp, Eucalyptus camaldulensis
ccaagaaatgatccagaaaaatccagattcactcaatttcaacgtcatagccctttctaa

SEQ ID NO: 1385 - DNA, 60 bp, Eucalyptus camaldulensis
aacgtttcctcagtcattcacagctgggtgaagaaagattctttccgcttcaggcagatt

SEQ ID NO: 1386 - DNA, 60 bp, Eucalyptus camaldulensis
ttttttgtagcattcacacattgatttgagagctgtatatcgatgccaatgatatggctt

SEQ ID NO: 1387 - DNA, 60 bp, Eucalyptus camaldulensis
tgtgagagagtatgattgattttgtcaattgggtatctttgatcttctagcaaatttcgg

SEQ ID NO: 1388 - DNA, 60 bp, Eucalyptus camaldulensis
ttagctacgtcagtttctgatgcttggactgcagggtgcggtgatgatcaatgtatcttt

SEQ ID NO: 1389 - DNA, 60 bp, Eucalyptus camaldulensis
tttggagtggacttttcacgagatcacagtattaacaaatgggaaaagagggcacatttt

SEQ ID NO: 1390 - DNA, 60 bp, Eucalyptus camaldulensis
agaggttgttcaattttaagaaagaccgtgatggatactggaaatggtttgctggtaact

SEQ ID NO: 1391 - DNA, 60 bp, Eucalyptus camaldulensis
tgtcttgggatgcttttgctgtggaagaatttacaatctgataaccttctattgcattat

SEQ ID NO: 1392 - DNA, 60 bp, Eucalyptus camaldulensis
gggtgtcgtcctcgttactaggaatttcgttgttcattttgtagctattggcaattgaca

SEQ ID NO: 1393 - DNA, 60 bp, Eucalyptus camaldulensis
ttccatgcttttttggtaccatggttgagcaagattgtcaggcgacctcggtttttgaga

SEQ ID NO: 1394 - DNA, 60 bp, Eucalyptus camaldulensis
ttcgacttggactttgccggagacatatttgcagatatacaattcgtcgttcttcctgca

SEQ ID NO: 1395 - DNA, 60 bp, Eucalyptus camaldulensis
gattattagtgtatttgatccaaaaagagcgatgcacttttaacgtcgtgctcggttctc

SEQ ID NO: 1396 - DNA, 60 bp, Eucalyptus camaldulensis
cagacctgtgggttcgcctcataattcttcgaattataatctggctttttcaatgaggcc

SEQ ID NO: 1397 - DNA, 60 bp, Eucalyptus camaldulensis
acgatcaccgagtcgtgttctgagatttcgtgcgtctttatctgagatttcacaactggt

SEQ ID NO: 1398 - DNA, 60 bp, Eucalyptus camaldulensis
tggaacacaatgtacataagagaaccaacacgcgtttgattgtttgctcaaggtcggtct

SEQ ID NO: 1399 - DNA, 60 bp, Eucalyptus camaldulensis
tgaggggtacgtattctggtgcagacaaatgcaattttgtacatctacatgctgttctgt

SEQ ID NO: 1400 - DNA, 60 bp, Eucalyptus camaldulensis
tgatcttgtagttaccaggcttgtaagtattcattcaactgtgcaagttcgctttcgctg

SEQ ID NO: 1401 - DNA, 60 bp, Eucalyptus camaldulensis
ttgtaattggtacagatctggtgtcgggtcgactaaattcaattgcatgcttttcatttt

SEQ ID NO: 1402 - DNA, 60 bp, Eucalyptus camaldulensis
gatcggtgtgatgtagagattcctttcgccgcttggtaattgacattctctcgcaaaaaa

SEQ ID NO: 1403 - DNA, 60 bp, Eucalyptus camaldulensis
gcatgtacgttgtaactccggaatctgacttccctgccatttcataaagctaactctgca

SEQ ID NO: 1404 - DNA, 60 bp, Eucalyptus camaldulensis
gaccgcaatgtcaacaagtctattgaccagctaccgctggcaaattcgttttcttctgaa

SEQ ID NO: 1405 - DNA, 60 bp, Eucalyptus camaldulensis
gtgagaacgtgaatgatgtcagctaagagctttgctcagtatgtttctatagaagcctat

SEQ ID NO: 1406 - DNA, 60 bp, Eucalyptus camaldulensis
tatataatcaatcctccgtaatgatccgtcctcagagccaatccttttcccgaggttacg

SEQ ID NO: 1407 - DNA, 60 bp, Eucalyptus camaldulensis
gtgtgatggatcttcatgtggtgcagctactctcacttgagtggtggattttcaagtcat

SEQ ID NO: 1408 - DNA, 60 bp, Eucalyptus camaldulensis
tttgttggagtagcactcgccttgcttggctggtgtaattgagaaatgactggtgtacat

SEQ ID NO: 1409 - DNA, 60 bp, Eucalyptus camaldulensis
ttcttgctttgggcttgaggaattatgacctctcattgacttcaatgtcggaaacttgaa

SEQ ID NO: 1410 - DNA, 60 bp, Eucalyptus camaldulensis
gtctaactggaaatcttgcacttcaacgaatgcaaaccttgtctgatacatctcctccct

SEQ ID NO: 1411 - DNA, 60 bp, Eucalyptus camaldulensis
atgtggttggacgaattacaatatcatcttacgttacgaacatgataatggaattgatgc

SEQ ID NO: 1412 - DNA, 60 bp, Eucalyptus camaldulensis
cgtgcgacaattgtgtagctgatgaatcattgacattgacaacatgacaacattgtgagt

SEQ ID NO: 1413 - DNA, 60 bp, Eucalyptus camaldulensis
ttttttccagcttgtattgaaactcagtcattgctccacgaatgaagtactctgcctgga

SEQ ID NO: 1414 - DNA, 60 bp, Eucalyptus camaldulensis
caattccactttgtacttgtcaaaacatttatctgcgcaaatgcaagatatttgaccgca

SEQ ID NO: 1415 - DNA, 60 bp, Eucalyptus camaldulensis
tctttatatgcgacctgcatgatgggatcggcaatctccttacatgtgtggatttccctt

SEQ ID NO: 1416 - DNA, 60 bp, Eucalyptus camaldulensis
ggtttcttcgtggaaggttgggatctaaaatattccttgagacatcgattgttggcttaa

SEQ ID NO: 1417 - DNA, 60 bp, Eucalyptus camaldulensis
ggtgttgaaccatatgatatagatgaagctggggccaactgcaaaaggcttgttcgcaaa

SEQ ID NO: 1418 - DNA, 60 bp, Eucalyptus camaldulensis
tctggatttttggaacaatgtgattttttccgtcagttgctatgtcccgacttttatctg

SEQ ID NO: 1419 - DNA, 60 bp, Eucalyptus camaldulensis
cggattgtacaatgatgaagctggtgtttatataatctcttcaaagtctcggatttgaaa

SEQ ID NO: 1420 - DNA, 60 bp, Eucalyptus camaldulensis
tgcaaaaggttttacgtctgtcatcatttatatgagcttcagactacaagtcttggtttt

SEQ ID NO: 1421 - DNA, 60 bp, Eucalyptus camaldulensis
ctttcgtgttgtttatccacccataagttgtgatacgcagatgcttgcgcccatcaatgt

SEQ ID NO: 1422 - DNA, 60 bp, Eucalyptus camaldulensis
gattttggagctcgaagttagctttttgaaatgattacttatgaaaccattttgctcgca

SEQ ID NO: 1423 - DNA, 60 bp, Eucalyptus camaldulensis
atgcaaatggtactattcgttgtacaccaatatactccagaatagtatccctgtttgcgg

SEQ ID NO: 1424 - DNA, 60 bp, Eucalyptus camaldulensis
acccatatctggtattaattacttctacacccatttaaggtgtctgtggtaatctggctt

SEQ ID NO: 1425 - DNA, 60 bp, Eucalyptus camaldulensis
tagtcagtttgctgacagctttgaaagaagccaatgccaaaatcgaacttgttcgtgtgc

SEQ ID NO: 1426 - DNA, 60 bp, Eucalyptus camaldulensis
tttcacccttggattttgtttggtataatgtcgacatttggcttcaaccaggctttttct

SEQ ID NO: 1427 - DNA, 60 bp, Eucalyptus camaldulensis
caaatttggccatgaaagtcaaggcagctggtgatagcggaatccatgtttaggaagcaa

SEQ ID NO: 1428 - DNA, 60 bp, Eucalyptus camaldulensis
ccaattgggactatttactaaatgtaatggatctgttaactctgtaagagaagttggttc

SEQ ID NO: 1429 - DNA, 60 bp, Eucalyptus camaldulensis
gatgaagatgctgacttggattctgaagtcgataatagcggcgactttgtagcggcaata

SEQ ID NO: 1430 - DNA, 60 bp, Eucalyptus camaldulensis
tgctattgggtttcaattttccttgtattttggtagtttgacaaaagagcggatctcaat

SEQ ID NO: 1431 - DNA, 60 bp, Eucalyptus camaldulensis
gtctctccggctggtgccctcgagtcccatcgacactgtctcgtctctctcttgatctcg

SEQ ID NO: 1432 - DNA, 60 bp, Eucalyptus camaldulensis
tgtccaggatcgagccaaatttgtactaagatggtgagaaaagtctggaaataagatttg

SEQ ID NO: 1433 - DNA, 60 bp, Eucalyptus camaldulensis
ttgttgtgaagcctgtgtgcaaaaacaggggttgactttcatcggcaactgttgaatcga

SEQ ID NO: 1434 - DNA, 60 bp, Eucalyptus camaldulensis
aatgcagtttcagttgccagtaccattttggggccttttttggcattagaatccggatca

SEQ ID NO: 1435 - DNA, 60 bp, Eucalyptus camaldulensis
ttaccgagttcgtgttggatgcagctggtaaaataagttgctgaaatgttgtttagaatt

SEQ ID NO: 1436 - DNA, 60 bp, Eucalyptus camaldulensis
agtgcttaggtctatcgtagaagctgtgttttgggtttatatattgtgtttacttttcac

SEQ ID NO: 1437 - DNA, 60 bp, Eucalyptus camaldulensis
gatgttgttatgcttttaaaaagaacgtaacttaatggatatgctttcaatcaaaaaaaa

SEQ ID NO: 1438 - DNA, 60 bp, Eucalyptus camaldulensis
attgggtgataccttttgtactgaaatgtttgtgtaaggtccatataaagcttcaaagag

SEQ ID NO: 1439 - DNA, 60 bp, Eucalyptus camaldulensis
tgtttcagttgtatgtctttgtcaataagaatacaagagtttggagtttcagttcagcgt

SEQ ID NO: 1440 - DNA, 60 bp, Eucalyptus camaldulensis
aaattgataggtggtttatggagatgatgcccaccaatgggtttttcacaagagccaagt

SEQ ID NO: 1441 - DNA, 60 bp, Eucalyptus camaldulensis
gctcatacagttgcagtggttctataaaacttagttgcaggtgaaacctccttgagggct

SEQ ID NO: 1442 - DNA, 60 bp, Eucalyptus camaldulensis
agatccgagatttgtaattttgaggaccatcgtgcccattttgattgcaattttgatata

SEQ ID NO: 1443 - DNA, 60 bp, Eucalyptus camaldulensis
cattatggccgggcacctgcagtgaaaattgtaggtatgcaagttctgacattcggagtt

SEQ ID NO: 1444 - DNA, 60 bp, Eucalyptus camaldulensis
ggtgctcttgtggaagagtaggcttctcgttttgtaccgttccgtgtcttgttctttttt

SEQ ID NO: 1445 - DNA, 60 bp, Eucalyptus camaldulensis
acgcaatgcccaggacaggttcccagtctatcaaatgtgaacaatcacagtgtttattac

SEQ ID NO: 1446 - DNA, 60 bp, Eucalyptus camaldulensis
tctcagattttaccgaaacattcatctgatgcattgtttatgaaattgtaccacaactgg

SEQ ID NO: 1447 - DNA, 60 bp, Eucalyptus camaldulensis
ttatcggcttgttactatgcaacagctgtttataaggactaggggcttgagtgcatgcct

SEQ ID NO: 1448 - DNA, 60 bp, Eucalyptus camaldulensis
gaggaattgttcggtatatattttgtctaatacataaatacatgagctacgacctacagg

SEQ ID NO: 1449 - DNA, 60 bp, Eucalyptus camaldulensis
tgaagtatagcaacatgtattcagctagagaagatgtacagatgtattataataccgtgg

SEQ ID NO: 1450 - DNA, 60 bp, Eucalyptus camaldulensis
ttgccatcaactatcgcgacattgcagtctagttgaatatagttatgtggtttgcatgaa

SEQ ID NO: 1451 - DNA, 60 bp, Eucalyptus camaldulensis
ttttgagctccagtttgatgaagatgagatccatgtaaactttcccttttgacgtttcga

SEQ ID NO: 1452 - DNA, 60 bp, Eucalyptus camaldulensis
gaaaccggaagatctaaattcttgtacttccaagaatgtcatttttgccaagacaatgtt

SEQ ID NO: 1453 - DNA, 60 bp, Eucalyptus camaldulensis
acattggaaataaccaccacatccgcacttcattgttctgcaggaagtgaaagtcattaa

SEQ ID NO: 1454 - DNA, 60 bp, Eucalyptus camaldulensis
tggaggcatgtatgaagaggatggagaactctcagttctaccagtgtcttgtggaacaaa

SEQ ID NO: 1455 - DNA, 60 bp, Eucalyptus camaldulensis
tactttcaaccttggtgtaataacgcatttcaagaagtgtaataaccttctgtggatcag

SEQ ID NO: 1456 - DNA, 60 bp, Eucalyptus camaldulensis
gccgttatgcaattattgtcaggattcatggtgaatctttgtgaacggatcatgtacaaa

SEQ ID NO: 1457 - DNA, 60 bp, Eucalyptus camaldulensis
caagactgcattttgttgtatcgtgattctgaaacctcagtattcgtgcggaaattgcct

SEQ ID NO: 1458 - DNA, 60 bp, Eucalyptus camaldulensis
agcactgaagaagctgaatctctcacgtcaaaataccgcgctgttgctgagcaatataaa

SEQ ID NO: 1459 - DNA, 60 bp, Eucalyptus camaldulensis
cttttgttctgttagttcttttgaacagacggtgaagactaatcatctatcatagagttg

SEQ ID NO: 1460 - DNA, 60 bp, Eucalyptus camaldulensis
aatactggcatctaacttgcagaggtgtagcccttgttataatcttggagttgctcttaa

SEQ ID NO: 1461 - DNA, 60 bp, Eucalyptus camaldulensis
ttagattgaatgagtaagattctggctacacagacctttaaatgatgtaatgattggctc

SEQ ID NO: 1462 - DNA, 60 bp, Eucalyptus camaldulensis
tagatttgttggatgtaaccttccatacattggagaggaattgggacattttacacccat

SEQ ID NO: 1463 - DNA, 60 bp, Eucalyptus camaldulensis
tctgccattcgacctccaatgattgtggagtggcattcttgactgcgttttcgaagttaa

SEQ ID NO: 1464 - DNA, 60 bp, Eucalyptus camaldulensis
agaagtcgggccagcaattgcctttgcaattagggaacaactgtatgagattgctatcca

SEQ ID NO: 1465 - DNA, 60 bp, Eucalyptus camaldulensis
agcggcacctcataggtaaaatgtttgatgagatgtttgacgtatttgattgtgacttga

SEQ ID NO: 1466 - DNA, 60 bp, Eucalyptus camaldulensis
tccattctgtcttggtataattcaaagaagcgaagctggtaagctatgttttattgcaat

SEQ ID NO: 1467 - DNA, 60 bp, Eucalyptus camaldulensis
agtcgatactgtttggtgatgcagtaaaaactttgtttcctattgccatggctgctttct

SEQ ID NO: 1468 - DNA, 60 bp, Eucalyptus camaldulensis
tgcagtaatcaagtagaatttacagagtccacttgacatggtttgattatgcagtaatca

SEQ ID NO: 1469 - DNA, 60 bp, Eucalyptus camaldulensis
acgggcatttttcccattaaattcaaagctttccaaatgtaaaatagggaattggattcc

SEQ ID NO: 1470 - DNA, 60 bp, Eucalyptus camaldulensis; identical to SEQ ID NO: 1214

SEQ ID NO: 1471 - DNA, 60 bp, Eucalyptus camaldulensis
gccagtaggaatgtgcacagatgaccatctgcgaacgatatcaatcttctgctaatacat

SEQ ID NO: 1472 - DNA, 60 bp, Eucalyptus camaldulensis
aacgataaatctatgtcggaatgctctttatggattgaatatgcttattctgtcatgctg

SEQ ID NO: 1473 - DNA, 60 bp, Eucalyptus camaldulensis
gggcgaaaaacaggtttggcttgaagtgtgattatgtagggacattgatccatcagagag

SEQ ID NO: 1474 - DNA, 60 bp, Eucalyptus camaldulensis
actgataccgtccgaaccatatatgctcatattttactatttcagttgatagtcaaagct

SEQ ID NO: 1475 - DNA, 60 bp, Eucalyptus camaldulensis
tgtttcctcaaattcccaactttgcttgtcatttgtgtcatgagacggaataaagtgggt

SEQ ID NO: 1476 - DNA, 60 bp, Eucalyptus camaldulensis
ttgcctccttgtactttggagcatgtatttgctactcatatataaagaacgtccctggtg

SEQ ID NO: 1477 - DNA, 60 bp, Eucalyptus camaldulensis
aggtttacagagactgtgagagccaggattgagaaagcaggtggtgaatgcttgacattt

SEQ ID NO: 1478 - DNA, 60 bp, Eucalyptus camaldulensis
ccttcgtgcagccagatggcgcttaaattaatctttcctctccctttttgttcttagcct

SEQ ID NO: 1479 - DNA, 60 bp, Eucalyptus camaldulensis
ctcggtgtttttgggttaatactgctcatcttttagtagatgaacctccttaatataatc

SEQ ID NO: 1480 - DNA, 60 bp, Eucalyptus camaldulensis
ttgctgtgtcttttcagtattttgatgtgcggcagctcaatgcttatggtcgaactctga

SEQ ID NO: 1481 - DNA, 60 bp, Eucalyptus camaldulensis
ctgtttgctgctggacccccttgaaatttttgtttactctaaatgagatgttttcgggac

SEQ ID NO: 1482 - DNA, 60 bp, Eucalyptus camaldulensis
tatcgaacgtgtagttggcatgcagatcatgacagtgaagccagactcttgcatcattga

SEQ ID NO: 1483 - DNA, 60 bp, Eucalyptus camaldulensis
gtatttgtctactacttttatcttgatcaatctacctgagtagtgtgggtgtcattcaca

SEQ ID NO: 1484 - DNA, 60 bp, Eucalyptus camaldulensis
gatgccaaagttaatgagatgaaggctatggtaaagttccaactcaagaaggtgctctgc

SEQ ID NO: 1485 - DNA, 60 bp, Eucalyptus camaldulensis
agcacattagagaacgaggtagatgaagagcaagggcaaacagagatgtcatcatcgact

SEQ ID NO: 1486 - DNA, 60 bp, Eucalyptus camaldulensis
gtataatcaaacaacacacgacatacatacatcaatcgtggcgatcaatctatgaacact

SEQ ID NO: 1487 - DNA, 60 bp, Eucalyptus camaldulensis
ggtagtgggtttttattttcagatgaaatgaacttgtgggtttagtttctggttctatgt

SEQ ID NO: 1488 - DNA, 60 bp, Eucalyptus camaldulensis
tgctgttgggaaggtccatgaataaagtacgtagatttgggttctgcttctacgtattat

SEQ ID NO: 1489 - DNA, 60 bp, Eucalyptus camaldulensis
tttcaggctgtaggactgtactgctgacttttgtatctttgccgccctatacacggaatt

SEQ ID NO: 1490 - DNA, 60 bp, Eucalyptus camaldulensis
gttcaagcagtggaatgttatgtggaccatattgtttgattccgcaatctatcaacttag

SEQ ID NO: 1491 - DNA, 60 bp, Eucalyptus camaldulensis
gttgtattgttggattcatttcttatatatgaaatggcgccctggctctgtcggccggtt

SEQ ID NO: 1492 - DNA, 60 bp, Eucalyptus camaldulensis
ggatttggtacttgccttagttggtgatcaactcgactatagtgatgacatatgtggtgc

SEQ ID NO: 1493 - DNA, 60 bp, Eucalyptus camaldulensis
aactgttcatatcaaagctacgacatgacgtcgcaacacttcaatctgttatggtcaaat

SEQ ID NO: 1494 - DNA, 60 bp, Eucalyptus camaldulensis
cttattcgtcatgatgtgacagagccattgctagttgaggttgatcaaatttaccatctt

SEQ ID NO: 1495 - DNA, 60 bp, Eucalyptus camaldulensis
tcaggatatctcatttgagggcaatccaagcagtgatcttgcgacttccacaacttctgt

SEQ ID NO: 1496 - DNA, 60 bp, Eucalyptus camaldulensis
gaaatttgccatgcctgtgacaatgaattgaccaagatctttgtgcttccaacaaccact

SEQ ID NO: 1497 - DNA, 60 bp, Eucalyptus camaldulensis
agccaactacacaacatttacattatgggttatggttcactgacattgtaatgcacaggt

SEQ ID NO: 1498 - DNA, 60 bp, Eucalyptus camaldulensis
tccgtagttcttgggatgcacctggtgaggattcgatgttttgttgatggttccttttgt

SEQ ID NO: 1499 - DNA, 60 bp, Eucalyptus camaldulensis
tcctccatctgttgtaacaatcttatatatggatgttttgatccaaaggttaatggtgtc

SEQ ID NO: 1500 - DNA, 60 bp, Eucalyptus camaldulensis
ccttcttgctgtgatggattgctcttaaacccttcctcagtataaatatatattcagaaa

SEQ ID NO: 1501 - DNA, 60 bp, Eucalyptus camaldulensis
ccatcgacaactccagtattttatatacattcagtttgggcggtgatgcattgagcaagt

SEQ ID NO: 1502 - DNA, 60 bp, Eucalyptus camaldulensis
gaaacagaggagatgcttgagttctgcaaagaaaagggattgacttcccagatcgaagtg

SEQ ID NO: 1503 - DNA, 60 bp, Eucalyptus camaldulensis
ttgatctttgttagcgaagcgagagctcttcaagagtatttatactgcagattaatggct

SEQ ID NO: 1504 - DNA, 60 bp, Eucalyptus camaldulensis
tcttagcagtggtttagccagacttacattaattgtatgccgagcttcatccagaaaact

SEQ ID NO: 1505 - DNA, 60 bp, Eucalyptus camaldulensis
cagaatgggaaattgagtttttcgtccgtctggttaaagacaatgcgccatcctctgaaa

SEQ ID NO: 1506 - DNA, 60 bp, Eucalyptus camaldulensis
ttcggctgttgtgccagatacaaatattatgtaaaccaaggttaattgcgtatgaacatg

SEQ ID NO: 1507 - DNA, 60 bp, Eucalyptus camaldulensis
tcagtctgtcaatccttactatgtctggacctggtaagtttccccgtgttgagtcgaatt

SEQ ID NO: 1508 - DNA, 60 bp, Eucalyptus camaldulensis
attatgccccaactgtgccgattattctggtgggcacaaaactggatttaagggaagaca

SEQ ID NO: 1509 - DNA, 60 bp, Eucalyptus camaldulensis
tatcggaacgattttgggaatcacccagttggtgttgtattgtcactataaacgcactgg

SEQ ID NO: 1510 - DNA, 60 bp, Eucalyptus camaldulensis
ctgatcgactgcgttaacatgactttccgagaataaatcagatccaaactagatgtgctc

SEQ ID NO: 1511 - DNA, 60 bp, Eucalyptus camaldulensis
ctacttcgatgacagccaatgttatcaacttttaagtagttcggctactaattgatgata

SEQ ID NO: 1512 - DNA, 60 bp, Eucalyptus camaldulensis
tccgcttttgcatcagagcgttggatcaccaatatatgcactcttaaattataacgcaca

SEQ ID NO: 1513 - DNA, 60 bp, Eucalyptus camaldulensis
tcccttggattctcggcgattctctcttttacatgccagagcttcttgttgtgctttgat

SEQ ID NO: 1514 - DNA, 60 bp, Eucalyptus camaldulensis
ataagtcgaaatttgtgggagacagaatcctggttaattatttgtatggacagacgccca

SEQ ID NO: 1515 - DNA, 60 bp, Eucalyptus camaldulensis
atgtaattagcgagagctcttaacatgcttaaatcctctgtttccggtcagtgatcgctg

SEQ ID NO: 1516 - DNA, 60 bp, Eucalyptus camaldulensis
tctcacctacctcactgaaacaatcttgatgttgatgcatccagaaatggatggtggttg

SEQ ID NO: 1517 - DNA, 60 bp, Eucalyptus camaldulensis
gtagtatttgcggagaaccatccaaacttttgagtgtatctcccgttgcaaatctgaggt

SEQ ID NO: 1518 - DNA, 60 bp, Eucalyptus camaldulensis
ggactgactagtgttgataccgtaatgttgtattcatcagagtcatattttccccgttca

SEQ ID NO: 1519 - DNA, 60 bp, Eucalyptus camaldulensis
ttgtgtggtgatggtaattggcaatgtacctcgtccattatgatgggaataaatttgctc

SEQ ID NO: 1520 - DNA, 60 bp, Eucalyptus camaldulensis
atgaatgtaatttcatcaccataatcttgggccacaaccgcggtccctgagaataaaagc

SEQ ID NO: 1521 - DNA, 60 bp, Eucalyptus camaldulensis
tctaatttatgggaaccttaaacttattcattggaatgcgaaatcttgcgcatgacgcgg

SEQ ID NO: 1522 - DNA, 60 bp, Eucalyptus camaldulensis
tggatttgtggagacaaattttttgtgcctctctcgttggaaacggcaattgatataaat

SEQ ID NO: 1523 - DNA, 60 bp, Eucalyptus camaldulensis
cagctgtaccataaccgtgtacatttgtcgacgcgtacatgcagttttgacaatgtgaaa

SEQ ID NO: 1524 - DNA, 60 bp, Eucalyptus camaldulensis
ttgtgattacgtgcgccttaagcatggatggcacttgtggtgttcgatggcttttactaa

SEQ ID NO: 1525 - DNA, 60 bp, Eucalyptus camaldulensis
tttcaattgaattttgaactctgatgatctcctgaagaaaatcaattgtgcccttgtcac

SEQ ID NO: 1526 - DNA, 60 bp, Eucalyptus camaldulensis
tatttgttgcttagtactgtaattgtcgggtttaaattgccaagatttgaaatctcctgc

SEQ ID NO: 1527 - DNA, 60 bp, Eucalyptus camaldulensis
tgtggtgcctatgattcgatattggtatggactatgattatgagcgtggttggtaaaaaa

SEQ ID NO: 1528 - DNA, 60 bp, Eucalyptus camaldulensis
cccatttcttcttttctgggatgtttcttctgtttgaaggtgaaaattctcttgatggcc

SEQ ID NO: 1529 - DNA, 60 bp, Eucalyptus camaldulensis
tagtggtcttagcttgacttaaccgataatggtaggcacgcaatgcggatttgactgaaa

SEQ ID NO: 1530 - DNA, 60 bp, Eucalyptus camaldulensis
tgtggagaacttattactgtttgttcagaaaatgttgctgatggcttatctaaggcgtgg

SEQ ID NO: 1531 - DNA, 60 bp, Eucalyptus camaldulensis
tgttgacgatctcatatttgaactttagaatgagtggaagttttgattttggcttcaagg

SEQ ID NO: 1532 - DNA, 60 bp, Eucalyptus camaldulensis
ggaacttgctcaacaaattgaaaaggtcatgcgggcccttggtgattatcttggtgtgaa

SEQ ID NO: 1533 - DNA, 60 bp, Eucalyptus camaldulensis
aaactgccaatgtcaagcagtgacatatttatagatggtgtacgatgatcggttgtccat

SEQ ID NO: 1534 - DNA, 60 bp, Eucalyptus camaldulensis
tgagcaactactgtgttcaatatgccaaatgcccagtgcttgtggtgaggaaaccagaat

SEQ ID NO: 1535 - DNA, 60 bp, Eucalyptus camaldulensis
cattgtttcactttgtaataaacatccaaccaatacgaaggaaaggaggtgttttaggaa

SEQ ID NO: 1536 - DNA, 60 bp, Eucalyptus camaldulensis
ggaaacagttgactctccaaaatcaaatgaaattccaaataagatcagtggcgatgatga

SEQ ID NO: 1537 - DNA, 60 bp, Eucalyptus camaldulensis
ttttttcttctgagagttgtttaaccttgataagttggaggcatttggcgcagcttcaat

SEQ ID NO: 1538 - DNA, 60 bp, Eucalyptus camaldulensis
attttctgtcgtgtatacccttcatttaatggcaccctgatgttttaacagtttcctgga

SEQ ID NO: 1539 - DNA, 60 bp, Eucalyptus camaldulensis
tagtcgagctcagcagtgaagccgcctacaagagctgcgacattggcaagagcgtcaact

SEQ ID NO: 1540 - DNA, 60 bp, Eucalyptus camaldulensis
ttctaaagttgtcatcttcagcaatgacgtggagtgcaagagtttcaccgtttctcctca

SEQ ID NO: 1541 - DNA, 60 bp, Eucalyptus camaldulensis
gtgcttcctcattctgtattataagaactcagaaggatttacttctggaccataactaca

SEQ ID NO: 1542 - DNA, 60 bp, Eucalyptus camaldulensis
ttggcagaaataggttatcttttgtgcagaggatttaggaaagacttataattatcgtcg

SEQ ID NO: 1543 - DNA, 60 bp, Eucalyptus camaldulensis
ggccttccaagatgatgttactggttatctgaatttgcgagatgatttatgtgatagtga

SEQ ID NO: 1544 - DNA, 60 bp, Eucalyptus camaldulensis
tgctgttctcctgttattcataatcgggattttgagccttatatttgtcttcctttgggg

SEQ ID NO: 1545 - DNA, 60 bp, Eucalyptus camaldulensis
tctgagcttatgggacacacatattagttgcttgactgttcttttgcaacattgtcgaat

SEQ ID NO: 1546 - DNA, 60 bp, Eucalyptus camaldulensis
tcgatccagtgaacagacgactgttatgaacattttaaggtgaagtttgctttgtggggt

SEQ ID NO: 1547 - DNA, 60 bp, Eucalyptus camaldulensis
ttagaggctataaattccattaaataggactgcagcagctttcacttcgtggttggtgca

SEQ ID NO: 1548 - DNA, 60 bp, Eucalyptus camaldulensis
aatgccattctgcagtaatttatccactgtacttgtttatgtaactggcctaggtatgtt

SEQ ID NO: 1549 - DNA, 60 bp, Eucalyptus camaldulensis
ggggttttataatacgttccgcttgcacattaataaaatgaattgggtcggcgacttaaa

SEQ ID NO: 1550 - DNA, 60 bp, Eucalyptus camaldulensis
tacggatgagaacagtaactggagaataaactgaagctttgactttggctttggctttgc

SEQ ID NO: 1551 - DNA, 60 bp, Eucalyptus camaldulensis
agctgcctgtgccttaagaattttcgttaatcaagacttccggttgatatttaaaaaaaa

SEQ ID NO: 1552 - DNA, 60 bp, Eucalyptus camaldulensis
ctctgacagagtcttttctgtattttgtggaagcaattaattgaatgactctcatttcct

SEQ ID NO: 1553 - DNA, 60 bp, Eucalyptus camaldulensis
ggctatgggttgcagatatatagttgtacaggtatcttttcaggaactttgactcgacca

SEQ ID NO: 1554 - DNA, 60 bp, Eucalyptus camaldulensis
cgatctcgtgatcatgtcatttcgatcacaaaataatcggagatctgcgagctgtagaat

SEQ ID NO: 1555 - DNA, 60 bp, Eucalyptus camaldulensis
atcgtactgatgaccgcaacttaagcaacttaattgatcaaaagaaggatttctcctgca

SEQ ID NO: 1556 - DNA, 60 bp, Eucalyptus camaldulensis
atgaaattcatggatgtttgatgagaaagtaaccgaaacctacttggctctaattgcatt

SEQ ID NO: 1557 - DNA, 60 bp, Eucalyptus camaldulensis
tgcaacagttagtggatcacttgaggggcatggggatgataacaacggagaaaatcacat

SEQ ID NO: 1558 - DNA, 60 bp, Eucalyptus camaldulensis
ttttttcagacacgaattgtgtctccattcaatcagatcaattttgtccctggatatcaa

SEQ ID NO: 1559 - DNA, 60 bp, Eucalyptus camaldulensis
ggaatggagaattccactgctgcctgttgcattgatctactctactacacttgtgcttca

SEQ ID NO: 1560 - DNA, 60 bp, Eucalyptus camaldulensis
agtgggaagctacgtgctcagcgtgctaaatcgatgaagtttaaggatgggtacatgata

SEQ ID NO: 1561 - DNA, 60 bp, Eucalyptus camaldulensis
atatttgttaagagtgggcttttcaccctttctcagaagaacacaggctctgttgtgttg

SEQ ID NO: 1562 - DNA, 60 bp, Eucalyptus camaldulensis
tctcctgagacgcgggttgatccttgaaacgttgtttgggtgctggattttcattatctt

SEQ ID NO: 1563 - DNA, 60 bp, Eucalyptus camaldulensis
atgaagcggctctgattagagtggttgttaccagggctgaggtcgacatgaagttaataa

SEQ ID NO: 1564 - DNA, 60 bp, Eucalyptus camaldulensis
agaactacatgttagctgcgattttcaaccccttatggagctgtcttttgtaacaagcac

SEQ ID NO: 1565 - DNA, 60 bp, Eucalyptus camaldulensis
agcagaagtttcacacatacggaattagagacgatcacagaagatcaggaagccgaggat

SEQ ID NO: 1566 - DNA, 60 bp, Eucalyptus camaldulensis
aattgtaagctcaacaaaatcccaaacagctgccccatccccagataggaattggtggtt

SEQ ID NO: 1567 - DNA, 60 bp, Eucalyptus camaldulensis
ttatgaaatgcctttcggatttttccaccttaagggccagtaattcggttcgtagttttc

SEQ ID NO: 1568 - DNA, 60 bp, Eucalyptus camaldulensis
tatattccaccggtgctgtaacacataagtcgatccgattccttgaattttgggagccaa

SEQ ID NO: 1569 - DNA, 60 bp, Eucalyptus camaldulensis
gcaactgtaaaagctgtacgagaaatcatgtatatacagactcaacggcatcattggtga

SEQ ID NO: 1570 - DNA, 60 bp, Eucalyptus camaldulensis
gcaattgctaggggtgtgataaattgaactaggaggaagctcacctgaaatgttagtttt

SEQ ID NO: 1571 - DNA, 60 bp, Eucalyptus camaldulensis
ttccccattgtggggagattcgaatctgccatgcctttttcttcgatgacgtagttgtta

SEQ ID NO: 1572 - DNA, 60 bp, Eucalyptus camaldulensis
cttaagatgccgtagagccagatctcgcttgtcgcaattgtgtgtcctgattgaatcttt

SEQ ID NO: 1573 - DNA, 60 bp, Eucalyptus camaldulensis
agctgttgccgccaaaatgaatccaccattggtccaagacttcaccttgccatgttattt

SEQ ID NO: 1574 - DNA, 60 bp, Eucalyptus camaldulensis
aaatatctcaacattgttggttgacttggggattttgtgactccgtagctgactataaat

SEQ ID NO: 1575 - DNA, 60 bp, Eucalyptus camaldulensis
tataattgccgggggctaatacagttgtgctcctctttcaaaagaaagtgttgggggagt

SEQ ID NO: 1576 - DNA, 60 bp, Eucalyptus camaldulensis
ccgttcagaccattagcccgaagaaaccacttaacaagcagggaagtaatgtcgctaaaa

SEQ ID NO: 1577 - DNA, 60 bp, Eucalyptus camaldulensis
caactgtcctgtctgcagcctttttgtctgatcggcagaatgtcatgttttgatcgtcaa

SEQ ID NO: 1578 - DNA, 60 bp, Eucalyptus camaldulensis
cgttattctttttcccaccgtggcatttgcatgcgtaaccgcaaattctcgtccatcatt

SEQ ID NO: 1579 - DNA, 60 bp, Eucalyptus camaldulensis
aaccaaattcagtgggatgtatttgtagtggcagagttgattgtgcaaagcttttggaat

SEQ ID NO: 1580 - DNA, 60 bp, Eucalyptus camaldulensis
ggatggggtcttgcttttctattgttatagtattttatgatcattgcaattgggttttgc

SEQ ID NO: 1581 - DNA, 60 bp, Eucalyptus camaldulensis
attgtcagtcttataccgagcaaggattttagaatggagcggacaaagtaaatggccatt

SEQ ID NO: 1582 - DNA, 60 bp, Eucalyptus camaldulensis
aaatgtgaaagctactgctctgttactaataaggatctcatgggaagcttccccataaaa

SEQ ID NO: 1583 - DNA, 60 bp, Eucalyptus camaldulensis
attggaatttttgttgcagatcgcgttagttctgtttggagaatgcatgcaccattaata

SEQ ID NO: 1584 - DNA, 60 bp, Eucalyptus camaldulensis
gttgcttttggaggtctaccaatggatatggtcacaatatgtgataatgtcacaacttgg

SEQ ID NO: 1585 - DNA, 60 bp, Eucalyptus camaldulensis
atgatgactttgtatctttctttatgagagccaaggctggcctcaaacctggtgggcttt

SEQ ID NO: 1586 - DNA, 60 bp, Eucalyptus camaldulensis
tgtggtctgaaaggcttcatgtatccattttgttcaactcgaaaggtgttggtcaccgtt

SEQ ID NO: 1587 - DNA, 60 bp, Eucalyptus camaldulensis
acattgcctttgtgctctttaattcattccaagttgtatgtgcccaagtttctcagaagt

SEQ ID NO: 1588 - DNA, 60 bp, Eucalyptus camaldulensis
aatgcgcctgttactgtttaccttgtaattagaggatctatgtttctattgtagtattgg

SEQ ID NO: 1589 - DNA, 60 bp, Eucalyptus camaldulensis
ttcgcacaatgtatgagtgccataatgttgtttttctgatatgcatatcggcgatgctcc

SEQ ID NO: 1590 - DNA, 60 bp, Eucalyptus camaldulensis
tttttagaagtacccgcgtaaatttagattgcttgacatagtcaattgtatccagccact

SEQ ID NO: 1591 - DNA, 60 bp, Eucalyptus camaldulensis
tgctgaaaatggtgctgggagaagggattgatgatgttaagcttgagcattgtggtatat

SEQ ID NO: 1592 - DNA, 60 bp, Eucalyptus camaldulensis
ttttgaagtgtcgtcattgttttagaagattttaagtatatcaattttagacattgtgtt

SEQ ID NO: 1593 - DNA, 60 bp, Eucalyptus camaldulensis
agtgttctgtttcagtttggacctgtcatttttccttgagtactttgttttcttcaaagg

SEQ ID NO: 1594 - DNA, 60 bp, Eucalyptus camaldulensis
accgactgtcccgttgatagatttcatgttctctcgtctgtaatttagtaggttttcctg

SEQ ID NO: 1595 - DNA, 60 bp, Eucalyptus camaldulensis
gtaatctcaatgacagagcgaaaatgtgggagtttagattgcaacagttttctttttgga

SEQ ID NO: 1596 - DNA, 60 bp, Eucalyptus camaldulensis
ctgaatagggttctgacttttcaaaaatcagttccatttaataaacggtgcttgatgttg

SEQ ID NO: 1597 - DNA, 60 bp, Eucalyptus camaldulensis
gtagtcttgtttggtggacgtgttgtcgtggtataatctcaagtattcccacctaatcga

SEQ ID NO: 1598 - DNA, 60 bp, Eucalyptus camaldulensis
gctagcttatgggagcttttatgggagcatctgtgatttatttaataaaatacaatgcag

SEQ ID NO: 1599 - DNA, 60 bp, Eucalyptus camaldulensis
ttgcgagcgtaataacattcggatttcggtgaataagttgacttgcaaagcgaaatattg

SEQ ID NO: 1600 - DNA, 60 bp, Eucalyptus camaldulensis
aggtagctctagatgaagtttgttcatgtaatgcattcatcaatctgtttccatgtccat

SEQ ID NO: 1601 - DNA, 60 bp, Eucalyptus camaldulensis
atcacgttcaaacctgagtctggatgaaaacatacaccttatccacctcttcttatgttt

SEQ ID NO: 1602 - DNA, 60 bp, Eucalyptus camaldulensis
tatggccgggagatcaggcagattttggctttaaatgcatcatattttctcaagactgga

SEQ ID NO: 1603 - DNA, 60 bp, Eucalyptus camaldulensis
tctgcctgtacctctgttgtaaaaccttgctctccctgtgaagaggattacgtggtttaa

SEQ ID NO: 1604 - DNA, 60 bp, Eucalyptus camaldulensis
ttgcgcacattagcctctttttaacttcaatctcttatcttgaagtcctttgaggtcatc

SEQ ID NO: 1605 - DNA, 60 bp, Eucalyptus camaldulensis
ggcaagcgtagtggagctaaatgtccgttttgtgccaagaaaccaaaagaggaatagtta

SEQ ID NO: 1606 - DNA, 60 bp, Eucalyptus camaldulensis
tggtagctgttgagaaccgtgaaaatagctgcagaaagtctttgaccgtcggtgtctatt

SEQ ID NO: 1607 - DNA, 60 bp, Eucalyptus camaldulensis
tattcttgatttcgtgtgctccacgatgtcagcaagaaatcgtggtcttcttgccccttt

SEQ ID NO: 1608 - DNA, 60 bp, Eucalyptus camaldulensis
tttgttatgctcatgcgtcgtactttggaacttgttgatcaatgatctgtaatgtgaagt

SEQ ID NO: 1609 - DNA, 60 bp, Eucalyptus camaldulensis
catcgtgttttggtgtaattcgatgagaaaactgaaacagtctcttgctagacattgaca

SEQ ID NO: 1610 - DNA, 60 bp, Eucalyptus camaldulensis
cgggaacgagagaggacgatatgttttctctatggagtattattactacggtcggctttg

SEQ ID NO: 1611 - DNA, 60 bp, Eucalyptus camaldulensis
tttccccccatcgaggacaattgtcatttttcttttgagtatgaattataactaggtttg

SEQ ID NO: 1612 - DNA, 60 bp, Eucalyptus camaldulensis
gacatgccctatggatcttgcttttgtgtgtgtgcatatttccacttcgtggtgcttctt

SEQ ID NO: 1613 - DNA, 60 bp, Eucalyptus camaldulensis
tcatggcgttggtttatgtacttatgagaagtctttgattttgatctttaatcgagactg

SEQ ID NO: 1614 - DNA, 60 bp, Eucalyptus camaldulensis
tgttgcaattctcatttcctagacagcacgactcttaatcacaatggggatccattaagt

SEQ ID NO: 1615 - DNA, 60 bp, Eucalyptus camaldulensis
gttgcgtttaaattttgcaggtatcagtcagatgacatttctctagctttcccatacctc

SEQ ID NO: 1616 - DNA, 60 bp, Eucalyptus camaldulensis
tggggcaactgactcgagtattttactactgcactaaggtgaagtagcatcctatagcaa

SEQ ID NO: 1617 - DNA, 60 bp, Eucalyptus camaldulensis
ttgtggtcaattttgtccgtgtcggccctaatatataaagatacatgccgatattcatca

SEQ ID NO: 1618 - DNA, 60 bp, Eucalyptus camaldulensis
atctgtgatgggcaggacagacacttcagccgcctcagcagcacaggctgatgaagaaga

SEQ ID NO: 1619 - DNA, 60 bp, Eucalyptus camaldulensis
gagaaggcactaattgcctttctatttccttgtatatttagtgagagtaggtagcagata

SEQ ID NO: 1620 - DNA, 60 bp, Eucalyptus camaldulensis
ctggagagaaatcatgatttgctgtttgttttaactaagctttatattcaaatcccgcac

SEQ ID NO: 1621 - DNA, 60 bp, Eucalyptus camaldulensis
ttttatgcctccagtgcttgtatttatccagaatttaagcagctggagaccaacgtgagc

SEQ ID NO: 1622 - DNA, 60 bp, Eucalyptus camaldulensis
ttatatggccgacataacgagttttcgtgtgctaataagtcaaattgaaactgtggtttt

SEQ ID NO: 1623 - DNA, 60 bp, Eucalyptus camaldulensis
agcttgaagctgcgaaagagcgaaacttccgacgcccgtctctgcattctctcgaagctg

SEQ ID NO: 1624 - DNA, 60 bp, Eucalyptus camaldulensis
ttttcaatatgatgtaattttgcctttgtatcaagtgatgttgccatttgactcgcgatg

SEQ ID NO: 1625 - DNA, 60 bp, Eucalyptus camaldulensis
cttccaattgcataattaactacctgacaatgtccatcgtgtgaatactgagctttgttt

SEQ ID NO: 1626 - DNA, 60 bp, Eucalyptus camaldulensis
gctttcaaatttgtattgtagttctcagcaaaaagttgatcaatatttgcaacggtcacg

SEQ ID NO: 1627 - DNA, 60 bp, Eucalyptus camaldulensis
agagaagcagagatgagaatggtgagcgaaaaaaccaatggtagagttgcctggttcagt

SEQ ID NO: 1628 - DNA, 60 bp, Eucalyptus camaldulensis
cctgttttaggatgtccaagagatgacaagtatttgtttcttatttaactccaatgcatg

SEQ ID NO: 1629 - DNA, 60 bp, Eucalyptus camaldulensis
tggaaattattagctcagtttatttttaatgaatttcattaatttcaggccgcctcggcc

SEQ ID NO: 1630 - DNA, 60 bp, Eucalyptus camaldulensis
taaaattttggtgaaaatgctaaaagcaccagctcattgatcaagttcatatgcatgtgg

SEQ ID NO: 1631 - DNA, 60 bp, Eucalyptus camaldulensis
atgcgagcttctgtagccgtgatgcttctcgggtgaaaattggttgtgcgatattattga

SEQ ID NO: 1632 - DNA, 60 bp, Eucalyptus camaldulensis
cagctcagtaaacagattatttagttattcctatccttgaatgataattacttctctttt

SEQ ID NO: 1633 - DNA, 60 bp, Eucalyptus camaldulensis
gaaagcatcctcaatggttctaatcttatattgtttcgccactgatgctctgggcgtgta

SEQ ID NO: 1634 - DNA, 60 bp, Eucalyptus camaldulensis
cccgtaagtatcgcgtatctttgtaatcgagcatttagaatttacaaaacttcgctgaaa

SEQ ID NO: 1635 - DNA, 60 bp, Eucalyptus camaldulensis
gggcatgtgctatcgtaactattgtattatgggatgcatcttcacgcctttactcaaggg

SEQ ID NO: 1636 - DNA, 60 bp, Eucalyptus camaldulensis
attgctgcctttccaaaggtagcttgattccttcatcaatttttgaaggttgacttgccc

SEQ ID NO: 1637 - DNA, 60 bp, Eucalyptus camaldulensis
tctttttcttttttgggatctgtaggcccacaattttgtatcgagacacttgtgcatctt

SEQ ID NO: 1638 - DNA, 60 bp, Eucalyptus camaldulensis
aaacggatctgacgattaactcataatgctcctggtgcaattgcaatactgcttcaagtt

SEQ ID NO: 1639 - DNA, 60 bp, Eucalyptus camaldulensis
ttcgaggatcattttgctgggattcttggagaaataaagagcaatatatagcaataatcg

SEQ ID NO: 1640 - DNA, 60 bp, Eucalyptus camaldulensis
ctttatggactaaacatgatgatgaaattgatgactgttggaagttgtgaccaaaaaaaa

SEQ ID NO: 1641 - DNA, 60 bp, Eucalyptus camaldulensis
tgtcggtcctctttgatgctcttaagacttgtaaacttatatgggttgttaccttccttt

SEQ ID NO: 1642 - DNA, 60 bp, Eucalyptus camaldulensis
tttcagactccacagttttgctttttgtgttatttccttggtctcagaacaattttaagg

SEQ ID NO: 1643 - DNA, 60 bp, Eucalyptus camaldulensis
tgtgcactttgaattgagttgtttcaatctctattctccaactgatgcttactatgaatg

SEQ ID NO: 1644 - DNA, 60 bp, Eucalyptus camaldulensis
gattgaattgccatgaaggcggtgggcaaacctgtttagccttacttgtcagcaatgtaa

SEQ ID NO: 1645 - DNA, 60 bp, Eucalyptus camaldulensis
catcttttagcaggtttgggttttaacctagaatcacaacaacttggccaattcgcccta

SEQ ID NO: 1646 - DNA, 60 bp, Eucalyptus camaldulensis
gttaactagttttttgtccagtgattgagttctgactaagtgatttgataatgcctgtag

SEQ ID NO: 1647 - DNA, 60 bp, Eucalyptus camaldulensis
attgagcgaattggtggactggattggattgtagtggtttattctggagcttgaccctct

SEQ ID NO: 1648 - DNA, 60 bp, Eucalyptus camaldulensis
atccaccggtttcaggcatgtattatcaccatgacttcatggtcggaatgagtggcaatt

SEQ ID NO: 1649 - DNA, 60 bp, Eucalyptus camaldulensis
gtgaggtcgcaattttggaacaagaaacgtttatgaagtgaatttgcggttactttttaa

SEQ ID NO: 1650 - DNA, 60 bp, Eucalyptus camaldulensis
catgcttgtaagcttcatgttatcttaacagttatgtatggcatgagcgcaatcattcac

SEQ ID NO: 1651 - DNA, 60 bp, Eucalyptus camaldulensis
tatcgatctgatacccttgggttgtctccagttgatagtgtaatgtccgcgaatgcctta

SEQ ID NO: 1652 - DNA, 60 bp, Eucalyptus camaldulensis
gcgtttgactgaatatgtattccaactcgagcattagctggatctcttttaggaatagtg

SEQ ID NO: 1653 - DNA, 60 bp, Eucalyptus camaldulensis
tgtcgatgctttctagctcgactgttaagcaacgcccagaagataacgatcgttatgtga

SEQ ID NO: 1654 - DNA, 60 bp, Eucalyptus camaldulensis
ccgataaggaggatggtacaccattaagtggtggctcattccctcctccattcccaccct

SEQ ID NO: 1655 - DNA, 60 bp, Eucalyptus camaldulensis
atcaacggttcctctcgtactaggttgaattactattgtgacactatcccggccataatg

SEQ ID NO: 1656 - DNA, 60 bp, Eucalyptus camaldulensis
tttcgaattttcatatgggggaaatgtcaaatgtttgctaggtggtctgtggcttatctg

SEQ ID NO: 1657 - DNA, 60 bp, Eucalyptus camaldulensis
tggttctgttgaactgccagtctgccattaactgctggaaaaataagatagggagctttt

SEQ ID NO: 1658 - DNA, 60 bp, Eucalyptus camaldulensis
tcaactattgtctacggaacatggcttttatattcttctcgacgtggattggtgcatact

SEQ ID NO: 1659 - DNA, 60 bp, Eucalyptus camaldulensis
tgtgtattatgactggctgccaattacagtaatgaaagtgcccaggtgcaatattgccaa

SEQ ID NO: 1660 - DNA, 60 bp, Eucalyptus camaldulensis
tcactcaattgacgttgacaaagtagtggcatttttatgcagcaattgcaaggtcttcat

SEQ ID NO: 1661 - DNA, 60 bp, Eucalyptus camaldulensis
tgagatttgaggcctctagtttgcaatgtgttttgactattattgtgattctattgtctc

SEQ ID NO: 1662 - DNA, 60 bp, Eucalyptus camaldulensis
tcctcgtcgtcgtcttccccagcttgggcgaactttttaactttgtaaaaggtagttgaa

SEQ ID NO: 1663 - DNA, 60 bp, Eucalyptus camaldulensis
ttggagcaatatatgcctgcctctactcttatgtttagaagatttggcctttgggcagtc

SEQ ID NO: 1664 - DNA, 60 bp, Eucalyptus camaldulensis
atatagggtcgcgaaattatagactccggcgaaggaatggcaagaccccgttcaaccgca

SEQ ID NO: 1665 - DNA, 60 bp, Eucalyptus camaldulensis
gcgcttgttagtgatttgattttgcatcatcagtatgtctggtttcttttatcggaccca

SEQ ID NO: 1666 - DNA, 60 bp, Eucalyptus camaldulensis
ctggcaataatccagctgacgcaacctgatcggtatgtaaaatcaagatgtggattgctt

SEQ ID NO: 1667 - DNA, 60 bp, Eucalyptus camaldulensis
acctgaagttgcaggatgcctatctacttcttcttctttgacttgctggctagagactga

SEQ ID NO: 1668 - DNA, 60 bp, Eucalyptus camaldulensis
agagaggacgatatgtttttctctatggagtattattactacggtcggctttgagggggt

SEQ ID NO: 1669 - DNA, 60 bp, Eucalyptus camaldulensis
tgaccagatacaatgggatcttaacgagattcagatgaaactagacaatgaaagagagga

SEQ ID NO: 1670 - DNA, 60 bp, Eucalyptus camaldulensis
gtttatttggaaggactattttgtcccttacatgactgcataccaatggacagcgtcggc

SEQ ID NO: 1671 - DNA, 60 bp, Eucalyptus camaldulensis
agtagttgctgattggagctattttaatgatcatcggcctagcagacttgccacaaagag

SEQ ID NO: 1672 - DNA, 60 bp, Eucalyptus camaldulensis
acttttggcctctgttatgtcaaacacagtatgcctgtaattctctgcccggccataatg

SEQ ID NO: 1673 - DNA, 60 bp, Eucalyptus camaldulensis
aagcgcaaacccgtgagattttcctcgtatacatcttcatttcactcttgtatatcggta

SEQ ID NO: 1674 - DNA, 60 bp, Eucalyptus camaldulensis
ttgggggtaagtttgtgtgttcaagacgctttaatctttggaatgtatgaaatactccat

SEQ ID NO: 1675 - DNA, 60 bp, Eucalyptus camaldulensis
tcctatcattgtgaagcagaattcaccaagtgttggattgttcacccaccaataaaaaag

SEQ ID NO: 1676 - DNA, 60 bp, Eucalyptus camaldulensis
agtcgtcaagtccagtaggaggggattctttttgctttttgttattggtgtgttgggtgc

SEQ ID NO: 1677 - DNA, 60 bp, Eucalyptus camaldulensis
agttctgctctccaacattgcttttttaggactagagtgtgatttgaaatggaggggatc

SEQ ID NO: 1678 - DNA, 60 bp, Eucalyptus camaldulensis
ccatgtcctgtagatgatgttacgtaataaagagtggctctttttcttttctgactttaa

SEQ ID NO: 1679 - DNA, 60 bp, Eucalyptus camaldulensis
aattgatctagtattctcttatttgagggttgtggcttttgagcgttgtttcactagttg

SEQ ID NO: 1680 - DNA, 60 bp, Eucalyptus camaldulensis
atgcagaatgtgtcacgctgaatgctatttgacaaaacaaattgtctccattaatttgac

SEQ ID NO: 1681 - DNA, 60 bp, Eucalyptus camaldulensis
gtcgtttgttggaactagcactgatgtaaggtcagggatatatgttaatgtaaaaattac

SEQ ID NO: 1682 - DNA, 60 bp, Eucalyptus camaldulensis
tttgatgtcatacgtcctgtattgctactgatcgaaaggctttaatactacagcttcttc

SEQ ID NO: 1683 - DNA, 60 bp, Eucalyptus camaldulensis
accttgagttggctttagcttgagctcttgtaaattgttgcaattagtcttctaataaag

SEQ ID NO: 1684 - DNA, 60 bp, Eucalyptus camaldulensis
attggaaaaccagattgagacacggcattcagtctctctttgcttgctgttctgtcccat

SEQ ID NO: 1685 - DNA, 60 bp, Eucalyptus camaldulensis
ggcggttctcgtctcatgcgatctctgtgatttcagcgttcacctcgtgattttataatt

SEQ ID NO: 1686 - DNA, 60 bp, Eucalyptus camaldulensis
ttgtgtaatgttgcttgagcatctcattctcgttctctcaggatacctccccaagagaca

SEQ ID NO: 1687 - DNA, 60 bp, Eucalyptus camaldulensis
attatgagctgtacttcatgcgttggaaattcccccactgtttggggataaatggccagt

SEQ ID NO: 1688 - DNA, 60 bp, Eucalyptus camaldulensis
attttcgacatgtcggcgctcttcaatttccattcatttctcacggtagtgctgctggta

SEQ ID NO: 1689 - DNA, 60 bp, Eucalyptus camaldulensis
ccgggaattcggccattatggccgggcgattccgaaagtgtatacaaagttatggattgg

SEQ ID NO: 1690 - DNA, 60 bp, Eucalyptus camaldulensis
attctcggctccttttcacgtggagcatcgagcttcaaggcttttggtgtcttcattttt

SEQ ID NO: 1691 - DNA, 60 bp, Eucalyptus camaldulensis
gtttttgactgctacacggatcatgtgagcagttaaatctgtgtgtctgtgggtgtgtgt

SEQ ID NO: 1692 - DNA, 60 bp, Eucalyptus camaldulensis
ttgtactctcaactgaacatgtaaaaatcaagatattagcccctttcacccttttgggga

SEQ ID NO: 1693 - DNA, 60 bp, Eucalyptus camaldulensis
acacttccctaacgaattacgtcattccggagcataagcttgtcatcctgtccgcaattt

SEQ ID NO: 1694 - DNA, 60 bp, Eucalyptus camaldulensis
ctttttctgtcttgacactggtgacttgaatgttatggatcacagtaatcttattgctct

SEQ ID NO: 1695 - DNA, 60 bp, Eucalyptus camaldulensis
acatcagcagtgagtgacatcaattttgaagttttgatttattgacattttgagggacca

SEQ ID NO: 1696 - DNA, 60 bp, Eucalyptus camaldulensis
tgaagggcacagaacacgagacctaaatggattgcatgctcttccagtgtttgatcaaga

SEQ ID NO: 1697 - DNA, 60 bp, Eucalyptus camaldulensis
ttgcgccctatatgtcctcaactgtaattgattcggacaaataggggcgaacagattttg

SEQ ID NO: 1698 - DNA, 60 bp, Eucalyptus camaldulensis
aattgtctcaaaacgatgagatctattgttgttaacacctactttgagtgaggtgaattc

SEQ ID NO: 1699 - DNA, 60 bp, Eucalyptus camaldulensis
ccttaccctttttgggttgctatgttaaaccaagaaaagtagtaaaaaggagcgatttca

SEQ ID NO: 1700 - DNA, 60 bp, Eucalyptus camaldulensis
gaacttgctttgctgcacttagattggaatttctgttatttatttattggttcggttctc

SEQ ID NO: 1701 - DNA, 60 bp, Eucalyptus camaldulensis
tcccctgaattcaagtccaatcagcttcctctttgcgacttattgttcctctgtttttaa

SEQ ID NO: 1702 - DNA, 60 bp, Eucalyptus camaldulensis
gcaggataatgtcttaccctttttaatcttgttgtttagcattgcgagttatactttctc

SEQ ID NO: 1703 - DNA, 60 bp, Eucalyptus camaldulensis
ttggtcagtctctgtccacagctgatggaaactttcgataagcccgttcacatttttatt

SEQ ID NO: 1704 - DNA, 60 bp, Eucalyptus camaldulensis
tttttgtgtgtcttgcctgtagcaagttctcgaaaatggaaggtctattcagaaatttct

SEQ ID NO: 1705 - DNA, 60 bp, Eucalyptus camaldulensis
ctgtaagcccagcatctgcatcaagatttccaaattagctatataaattactgtgatatg

SEQ ID NO: 1706 - DNA, 60 bp, Eucalyptus camaldulensis
ttgtattgcattataagaagttttgtaagcccatatctcgctgatatgctcttatgccct

SEQ ID NO: 1707 - DNA, 60 bp, Eucalyptus camaldulensis
aatggatgttatgcagctgaaacttttaagtttattgctttgcgtaggagacagcctcct

SEQ ID NO: 1708 - DNA, 60 bp, Eucalyptus camaldulensis
atcctgttctgcgcatgttttagctatctatttcatggtcttgagcaataaaggaatgta

SEQ ID NO: 1709 - DNA, 60 bp, Eucalyptus camaldulensis
ttgatgtactgacacagcgtaatagtctacaactactgtttaagcaagcgggccattctc

SEQ ID NO: 1710 - DNA, 60 bp, Eucalyptus camaldulensis
atgtggtccatgcccctttttgagggacagaactattaatgttatgtaactgtacaaagc

SEQ ID NO: 1711 - DNA, 60 bp, Eucalyptus camaldulensis
ttgtccagagttttgttctcgaattttgatcaacttatggttagcacttgtgtatcgaga

SEQ ID NO: 1712 - DNA, 60 bp, Eucalyptus camaldulensis
gtcgtgaatgtttgagcttcacatctttgactaaactgaccgcaaccacaaaggcatgac

SEQ ID NO: 1713 - DNA, 60 bp, Eucalyptus camaldulensis
ggatccaattttttcgagcttttacttatgccaatggtctctggccttttcctctgatct

SEQ ID NO: 1714 - DNA, 60 bp, Eucalyptus camaldulensis
aaatgcatggctagacatggctgtgggtggtgccaagatacaggaagcatatctcatttt

SEQ ID NO: 1715 - DNA, 60 bp, Eucalyptus camaldulensis
ttatgtaacggggtcggatgttggtttctgcttattgcgaatcgcaaatgttttccgggt

SEQ ID NO: 1716 - DNA, 60 bp, Eucalyptus camaldulensis
tcaatttctaagtggattcttcctccacgctctgtacagtgacgactcgttcattgcgat

SEQ ID NO: 1717 - DNA, 60 bp, Eucalyptus camaldulensis
ggtgtaattatagacgctcctgattaaacatctctttggcagtgaacggtcaatagttga

SEQ ID NO: 1718 - DNA, 60 bp, Eucalyptus camaldulensis
tacgtgcttcgtgatttttcattgattttggtggtctggaatcttcctgtgtgggctcta

SEQ ID NO: 1719 - DNA, 60 bp, Eucalyptus camaldulensis
tgggtctatccaaatttgtttcggatgaacacggcaactggagcccctcctgattatttt

SEQ ID NO: 1720 - DNA, 60 bp, Eucalyptus camaldulensis
gtacctgtggatatcccggtggtttctttgaatgatgaaggaagaatggtgatggtctct

SEQ ID NO: 1721 - DNA, 60 bp, Eucalyptus camaldulensis
catacgtgtaaagagtgtgtgtttcattttgcgagtcgatcatactattcttgtaaaccc

SEQ ID NO: 1722 - DNA, 60 bp, Eucalyptus camaldulensis
taacgtttcaaagttttctttttggagctattctgaataatttctcgttgtctcctgacg

SEQ ID NO: 1723 - DNA, 60 bp, Eucalyptus camaldulensis
tttcctggccgttctgttttatgtaaataagtgtgatacaggtcatgctctatactacca

SEQ ID NO: 1724 - DNA, 60 bp, Eucalyptus camaldulensis
atttgacctgcgaacacattgtaatttcatcccctccattcgcgttaaacgcctctatga

SEQ ID NO: 1725 - DNA, 60 bp, Eucalyptus camaldulensis
atttcggtgctcgaccgattgaattttaatgatgattagtagagatcatgggaaaaaaaa

SEQ ID NO: 1726 - DNA, 60 bp, Eucalyptus camaldulensis
ctcctggatgcttcgtgtgttctgcaacttgtaatgaaaggccttaatagggcgtgtttt

SEQ ID NO: 1727 - DNA, 60 bp, Eucalyptus camaldulensis
gattgaattgaattctgggaagtggttcttactaaagagcaaagcacgatcagacgacct

SEQ ID NO: 1728 - DNA, 60 bp, Eucalyptus camaldulensis
tcgtttagatgacttagattcttactttttaattctcacccatgagtgaagcgcagtctg

SEQ ID NO: 1729 - DNA, 60 bp, Eucalyptus camaldulensis
atgtcaattaggaaaggacagttatctgtctacttttgtggctagacctttgcttttcta

SEQ ID NO: 1730 - DNA, 60 bp, Eucalyptus camaldulensis
caatgagctcctgaatggaaagaggtcatgttgagtcagtgttatatcttgttgttgcac

SEQ ID NO: 1731 - DNA, 60 bp, Eucalyptus camaldulensis
tgcaatttgtttcaagacttgatggcttcaaccctagtggaaagaaatgaatttcatttc